(19) United States
(12) Patent Application Publication
(10) Pub. No.: US 2015/0376637 A1
(43) Pub. Date: Dec. 31, 2015

(54) METHODS AND MEANS FOR INCREASING STRESS TOLERANCE AND BIOMASS IN PLANTS

(71) Applicants: THE UNIVERSITY COURT OF THE UNIVERSITY OF GLASGOW, Glasgow (GB); BAYER CROPSCIENCE LP, Durham, NC (US)

(72) Inventors: Anna AMTMANN, Glasgow (GB); Matthew HANNAH, Gent (BE); Veronique GOSSELE, Mater (BE); Manuel LOPEZ-VERNAZA, Maynooth, Co. Kildare (IE); Giorgio PERRELLA, Glasgow (GB); Christoph VERDUYN, Sint-Niklaas (BE)

(21) Appl. No.: 14/764,508

(22) PCT Filed: Jan. 27, 2014
(86) PCT No.: PCT/EP2014/051522

§ 371 Date: Jul. 29, 2015

Related U.S. Application Data
(60) Provisional application No. 61/757,899, filed on Jan. 29, 2013.

(30) Foreign Application Priority Data
Jan. 29, 2013 (EP) 13153013.1
Jul. 15, 2013 (EP) 13176512.5

(51) Int. Cl. C12N 15/82; C12N 9/80

(52) U.S. Cl. CPC C12N 15/8261; C12N 9/80; C12N 15/8273; C12N 15/827; C12Y 305/01098

(57) ABSTRACT

The invention provides methods for producing a plant with increased stress-tolerance and yield, as well as chimeric genes for use according to the methods and plant comprising such chimeric genes.

### FIELD OF THE INVENTION

The present invention relates generally to the field of plant molecular biology and concerns a method for improving plant tolerance to stress conditions. More specifically, the present invention concerns a method for increasing stress tolerance and growth and for reducing ABA sensitivity, comprising increasing the expression and/or activity of a HISTONE DEACETYLASE COMPLEX 1 (HDC1) protein in a plant. The present invention also concerns plants having an increased expression and/or activity of HDC1, which plants have inter alia an increased stress tolerance, biomass, yield and reduced ABA sensitivity relative to corresponding wild-type plants. The invention also provides chimeric genes, nucleic acids and polypeptides encoding such HDC1 proteins.

### BACKGROUND

Population growth and climate change threaten to cause water scarcity and food shortage in many parts of the world (Lobell et al., 2011, Science 333, 616-620). There is an urgent need to increase yield, water usage efficiency and stress tolerance of food crops (Foresight, 2011, Final Project Report: Futures. Government Office for Science, London). A detailed understanding of the molecular entities that underpin plant responses to environmental stress is an essential prerequisite for crop improvement programs. Over the last two decades plant scientists have identified many pieces of the complex signalling network that regulates plant responses to environmental stresses (Cramer et al., 2011, BMC Plant Biol. 11.). The ‘stress’ hormone abscisic aid (ABA) masterminds a myriad of physiological and metabolic responses that protect the plant during periods of drought, salinity or freezing stress, and during seed maturation and dormancy (Yamaguchi-Shinozaki and Shinozaki, 2006, Annual Review of Plant Biology 57, 781-803; Urano et al., 2009, Plant J. 57, 1065-1078; Kim et al., 2010, In Annual Review of Plant Biology, Vol 61 (Palo Alto: ANNUAL REVIEWS), pp. 561-591; Yang et al., 2010, Mol Plant 3, 469-490). For example, ABA induces the closure of stomatal pores to minimise transpirational water loss and initiates the production of proteins and metabolites that prevent cellular damage during drying, thawing and osmotic shock. Cross-talk between ABA and other hormones such as ethylene (ET), gibberellin (GA), cytokinin (CK) and jasmonic acid (JA) integrates physiological and metabolic responses with plant growth and development (Chinnusamy et al., 2004, Journal of Experimental Botany 55, 225-236; Achard et al., 2006, Science 311, 91-94; Daszkowska-Golec, 2011, Omics 15, 763-774; Wilkinson et al., 2012, Journal of Experimental Botany 63, 3499-3509). The sophistication of hormonal signalling in plants was an evolutionary success but it often limits crop production because it makes plants unnecessarily ‘cautious’ in an environment that is largely controlled by the farmer. Thus, growth arrest and senescence, induced by the plant as pre-emptive measures to protect water and nutrient reserves during stress periods, can lead to yield penalties (Skirycz and Inze, 2010, Curr Opin Biotech 21, 197-203). There is now convincing evidence that growth reduction under water deficit is not a necessary consequence of stomatal closure but an active response of the plant, achieved by uncoupling growth from carbon signaling (Muller et al., 2011, Journal of Experimental Botany 62, 1715-1729). This means that maintaining biomass production with reduced water input is not a biological impossibility, and could be achieved by modulation of the natural hormone response of the plant. The validity of this approach was recently exemplified for CK, which induces senescence under water stress. If this response was suppressed by over-expression of a CK-biosynthesis enzyme yield under water-limited conditions was increased (Peleg et al., 2011, Plant Biotechnol J 9, 747-758). Similarly, reducing ABA-sensitivity and hence growth inhibition, or uncoupling ABA-induced protective measures from growth inhibition could be promising biotechnological approaches to obtain more ‘crop per drop’.

Many components of the ABA-signaling network have been identified including transcription factors, protein kinases/phosphatases, E3 ligases and small RNAs that act as positive or negative regulators (Hirayama and Shinozaki, 2007, Trends in Plant Sci. 12, 343-351; Sunkar et al., 2007, Trends in Plant Sci. 12, 301-309; Cutler et al., 2010, In Annual Review of Plant Biology, Vol 61 (Palo Alto: ANNUAL REVIEWS), pp. 651-679; Yang et al., 2010, supra). At a higher level of control, chromatin remodelling has emerged as an important factor for transcriptional responses to ABA (Chinnusamy et al., 2008, J lntegr Plant Biol 50, 1187-1195). For example, nucleosome assembly proteins and subunits of SWI/SNF chromatin-remodeling complexes have been reported to alter ABA sensitivity (Saez et al., 2008, Plant Cell 20, 2972-2988; Liu et al., 2009, Mol Plant 2, 688-699). Histone deacetylation (HD) has emerged as an important regulatory process during environmental stress (Kim et al. 2012, Plant Cell Physiol 53: 797-800). Histone de-acetylases (HDACs) remove active acetylation marks from lysine residues of histones 3 and 4 which in turn leads to repression of gene transcription both through interaction with gene-specific repressors and through general chromatin compression (Kurdistani and Grunstein, 2003, Nat Rev Mol Cell Bio 4, 276-284). In plants, HDACs belong to three different structural groups; Type-I HDACs, similar to Rpd3/HDAC1-type enzymes in yeast and animals, Sirtuins, homologous to similar enzymes in other eukaryotes, and HD-tuins, a plants specific class of proteins (Pandey et al. 2002, Nucleic Acids Res 30: 5036-5055; Hollender and Liu, 2008, J Integr Plant Biol 50, 875-885). The A. thaliana genome contains some twenty genes encoding HDACs only few of which have been functionally characterized. Over-expression of HD-tuin HD2C was reported to overcome ABA-induced growth arrest of germinating A. thaliana seeds (Sridha and Wu, 2006, Plant J. 46, 124-133). Conversely, seedlings of hd2c knockout mutants are ABA-hypersensitive as are seedlings of knockdown lines (axe1-5, CS2483) for HDA6, a Rpd3/HD1-type HDAC (Sridha and Wu, 2006, supra; Luo et al., 2012, Journal of Experimental Botany 63, 3297-3306, Chen et al. 2010, Exp Bot 61: 3345-3353). It was further shown that HD2C interacts with HDA6, and that crossing of axe1-5 with hd2c further increases ABA-sensitivity of seedlings (Luo et al., 2012, supra). The link between ABA-sensitivity, histone (de-)acetylation and transcriptional regulation was further strengthened by the finding that acetylation of H3/H4 lysine residues was increased and expression of many genes was modulated in knockdown/knockout lines for HD2C and HDA6 (To et al., 2011, PLoS Genet. 7; Luo et al., 2012, supra). However, not all HDACs function in ABA-signaling. For example, the function of A. thaliana HDA19 is more closely related to the defense hormone jasmonic acid. Knockout of HDA19 in A. thaliana caused a decrease in plant resistance to the fungal pathogen Alternaria brassicola. Over-expression of HDA19 had the opposite effect (increased resistance) but led also to developmental phenotypes (aberrant cotyledons, narrower, branching rosette leaves, delayed flowering, stunted siliques; Zhou et al. 2005, Plant cell 17: 1196-1204). Similarly, inducible over-expression of HDAC1-3 in rice caused developmental aberrations alongside enhanced growth (Jang et al. 2003, Plant J 33:531-541).

In yeast and animals, histone Rpd3/HD1-type histone de-acetylases act in conjunction with gene-specific transcriptional repressors (e.g. Ume6), a co-repressor (Sin3), Sin3-associated peptides (e.g. SAP18), histone-binding proteins (e.g. Ume1, RbAp46/48, TBL1) as well as functionally uncharacterised proteins (e.g. Rxt1-3) (Carrozza et al., 2005, Bba-Gene Struct Expr 1731, 77-87; Chen et al. 2012, Curr Biol 22: 56-63; Roguev and Krogan, 2007, Nat. Struct. Mol. Biol. 14, 358-359; Yang and Seto, 2008, Nat Rev Mol Cell Bio 9, 206-218.). Several types of complexes have been described each containing a distinct set of proteins. For example, yeasts assemble a large and a small Sin3 complex (Rpd3L/S in S. cerevisiae, I/II in S. pombe (Roguev and Krogan, 2007, supra) while mammals and insects assemble at least three distinct complexes (Mi-2/NuRD, CoREST and N-CoR/SMRT (Yang and Seto, 2008, supra). Recent experiments have shown that the protein environment of the catalytic histone de-acetylase enzymes in the complex is critical for the specificity of HD inhibitors (Bantscheff et al. 2011, Nature Biotech 29: 255-256). It is therefore likely that regulation of HDACs in vivo is similarly dependent on complex context. A few A. thaliana proteins with homology to members of animal or yeast HDAC complexes Sin3, SAP18, and the Rb46/48 homologue FVE have been characterized and found to interact with Rpd3/HD1-type histone de-acetylases HDA6 or HDA19 (Song et al., 2005, Plant Cell 17, 2384-2396; Song and Galbraith, 2006, Plant Mol. Biol. 60, 241-257;). Knockout/knockdown of these genes in A. thaliana caused similar phenotypes as knockdown of HDA6, e.g. ABA-hypersensitivity and delayed flowering (Song et al., 2005, supra; Song and Galbraith, 2006, supra). By, contrast, knockout of an A. thaliana homologue of mammalian TBL1 (HOS15) did not alter ABA-sensitivity but caused hypersensitivity of seedlings to cold (Zhu et al., 2008, Proc. Natl. Acad. Aci. USA 105, 4945-4950). These findings indicate that in plants HDACs also function in multi-protein complexes, but they also show that the physiological downstream responses of modifying putative complex members cannot be predicted from sequence homology alone. Clearly, many other HD complex proteins remain to be discovered and to be functionally characterized. Assembling putative plant HD complexes in silico is difficult because most yeast/animal HD complex proteins have either no or multiple homologues in the A. thaliana genome In total, over 100 A. thaliana genes have significant similarity to HDAC complex members in yeast or animals. Given the importance of HDACs in development and stress responses it is reasonable to assume that the specific composition and function of HDAC complexes depends on tissue, developmental stage and environment. WO04/022735 discloses proteins OsHDAC1, OsHDAC2 and OsHDAC3, which function as histone deacetylase, a gene coding for said proteins, and a method for producing a plant having a high growth rate by expressing said gene in the plant. Jang et al. (2003, supra) discloses that, while constitutive over-expression of HDAC1-3 in rice resulted in calli which could not be propagated, inducible overexpression also caused developmental aberrations in addition to enhanced growth.

WO04/035798 discloses a method for altering characteristics of a plant and describes the identification of genes that are upregulated or downregulated in transgenic plants overexpressing E2Fa/DPa and the use of such sequences to alter plant characteristics.

The present invention provides a contribution over the art by disclosing a new HDAC-interacting protein that can be used to modulate plant stress response, ABA-sensitivity, growth and flowering.

### SUMMARY OF THE INVENTION

In a first embodiment, the invention provides a method for increasing tolerance of a plant, plant part, plant organ or plant cell to stress conditions, preferably mild or moderate stress conditions; or for reducing ABA sensitivity of a plant, plant part, plant organ or plant cell; and/or for increasing biomass and/or yield and/or growth rate of a plant, plant organ or plant part; and/or for accelerating flowering time of a plant; comprising the step of
a. increasing the expression and/or activity of a protein having the activity of the protein with the amino acid sequence of SEQ ID NO. 6, in said plant, plant part, plant organ or plant cell.

Said increasing the expression and/or activity of a protein having the activity of the protein with the amino acid sequence of SEQ ID NO. 6 may comprise expressing in said plant cell, plant part, plant organ or plant a chimeric gene comprising the following operably linked elements:
a. A plant-expressible promoter
b. A nucleic acid which when transcribed results in an increased activity and/or expression of a protein having the activity of the protein encoded by SEQ ID NO. 6
c. Optionally, a 3′ end region involved in transcription termination and polyadenylation functional in plants

In a further embodiment of the method, the nucleic acid encodes a protein having the activity of the protein with the amino acid sequence of SEQ ID NO. 6, or the nucleic acid comprises a nucleic acid sequence encoding a protein having at least 70% sequence identity to SEQ ID NO. 6, SEQ ID NO. 8, SEQ ID NO. 10, SEQ ID NO. 12, SEQ ID NO. 14, SEQ ID NO. 16, SEQ ID NO. 18, SEQ ID NO. 20, SEQ ID NO. 22, SEQ ID NO. 24, SEQ ID NO. 26, SEQ ID NO. 28, SEQ ID NO. 30, SEQ ID NO. 32, SEQ ID NO. 34, SEQ ID NO. 36, SEQ ID NO. 38, SEQ ID NO. 40 or SEQ ID NO. 41, or the nucleic acid comprises a nucleic acid sequence having at least 70% sequence identity to SEQ ID NO. 5, SEQ ID NO. 7, SEQ ID NO. 9, SEQ ID NO. 11, SEQ ID NO. 13, SEQ ID NO. 15, SEQ ID NO. 17, SEQ ID NO. 19, SEQ ID NO. 21, SEQ ID NO. 23, SEQ ID NO. 25, SEQ ID NO. 27, SEQ ID NO. 29, SEQ ID NO. 31, SEQ ID NO. 33, SEQ ID NO. 35, SEQ ID NO. 37 or SEQ ID NO. 39.

The promoter may be a constitutive promoter or an inducible promoter.

In an even further embodiment, the plant is selected from wheat, oilseed rape, lettuce, tobacco, cotton, corn, rice, vegetable plants, carrot, cucumber, leek, pea, melon, potato, tomato, sorghum, rye, oat, sugarcane, peanut, flax, bean, sugar beets, soy bean, sunflower and ornamental plants.

The stress condition can be selected from drought stress, salt stress, low nutrient levels, high light stress and oxidative stress.

The invention furthermore provides a method for enhancing survival of a plant, plant part, plant organ or plant cell under severe stress conditions, or for enhancing recovery after severe stress of a plant, plant part, plant organ or plant cell, or for delaying the flowering time of a plant, comprising the step of:
a. decreasing the expression and/or activity of a protein having the activity of the protein encoded by SEQ ID NO.6 in said plant, plant part, plant organ or plant cell.

The reducing the expression and/or activity may comprise expressing in said plant cell, plant part, plant organ or plant a chimeric gene comprising the following operably linked elements:
a. A plant-expressible promoter
b. A nucleic acid which when transcribed results in a decreased activity and/or expression of a protein having the activity of the protein encoded by SEQ ID NO. 6
c. Optionally, a 3′ end region involved in transcription termination and polyadenylation functional in plants

In a further embodiment, the nucleic acid may when transcribed yield an HDC1 inhibitory RNA molecule.

Preferably, the promoter is an inducible promoter.

The invention also provides a chimeric gene as described above.

Also provided is a plant, plant part, plant organ, plant cell or seed that has been modified according to the invention so as to have an increased or reduced expression and/or activity of a protein having the activity of the protein with the amino acid sequence of SEQ ID NO. 6, when compared to a control plant, such as a plant, plant part, plant organ, plant cell or seed comprising a chimeric gene according to the invention.

The plant, plant part, plant organ, plant cell or seed of the invention can be oilseed rape, lettuce, tobacco, cotton, corn, rice, wheat, vegetable plants, carrot, cucumber, leek, pea, melon, potato, tomato, sorghum, rye, oat, sugarcane, peanut, flax, bean, sugar beets, soya, sunflower or ornamental plants.

Also provided is a method for reducing yield penalty of a plant under stress conditions, such as mild or moderate stress conditions, comprising increasing in said plant the expression and/or activity of a protein having the activity of the protein with the amino acid sequence of SEQ ID NO. 6., for example by expressing in said plant a chimeric gene as described above for increasing the activity and/or expression of a protein having the activity of the protein encoded by SEQ ID NO. 6 (i.e. the chimeric gene comprising a nucleic acid which when transcribed results in an increased activity and/or expression of a protein having the activity of the protein encoded by SEQ ID NO. 6 operably linked to a plant-expressible promoter and optionally a plant-functional a 3′ end region).

Further provided is a method for producing a plant with increased tolerance to stress conditions, such as mild or moderate stress conditions, or a plant with reduced ABA sensitivity, or a plant with increased biomass or yield or growth rate, or a plant with an earlier flowering time, comprising the steps of:
a. Introducing into a cell of a plant a chimeric gene as described above for increased activity and/or expression of a protein having the activity of the protein encoded by SEQ ID NO. 6 to generate a transgenic cell; and
b. Generating a plant, plant part, plant organ from said transgenic plant cell expressing said chimeric gene.

The invention also provides a method for modulating histone acetylation in a cell, comprising the step of modulating the expression and/or activity of a protein having the activity of the protein encoded by SEQ ID NO. 6 in said cell, wherein increasing the expression and/or activity of said protein inhibits histone acetylation and decreasing the expression and/or activity of said protein enhances histone acetylation.

Further provided is the use of a chimeric gene as described above for increased activity and/or expression of a protein having the activity of the protein encoded by SEQ ID NO. 6 to increase the tolerance of a plant, plant part, plant organ or plant cell to (mild or moderate) stress conditions; or to reduce ABA sensitivity of a plant, plant part, plant organ or plant cell; or to increasing biomass or yield or growth rate of a plant, plant organ or plant part; or to accelerate flowering time of a plant. Use the plant of claim 14 or 15, to produce seed comprising the chimeric gene of claim 13.

The invention also provides the use of a plant which has been modified so as to have an increased expression and/or activity of a protein having the activity of the protein with the amino acid sequence of SEQ ID NO. 6., for instance of a plant comprising a chimeric gene as described above for increasing the activity and/or expression of a protein having the activity of the protein encoded by SEQ ID NO. 6, to produce a population of plants with increased tolerance to (mild or moderate) stress conditions, or with reduced ABA sensitivity, or with increased biomass or yield or growth rate, or with an accelerated flowering time.

In another embodiment, the invention provides a protein having the activity of the protein with the amino acid sequence of SEQ ID NO. 6. That protein may have at least 70% sequence identity to SEQ ID NO. 6, SEQ ID NO. 8, SEQ ID NO. 10, SEQ ID NO. 12, SEQ ID NO. 14, SEQ ID NO. 16, SEQ ID NO. 18, SEQ ID NO. 20, SEQ ID NO. 22, SEQ ID NO. 24, SEQ ID NO. 26, SEQ ID NO. 28, SEQ ID NO. 30, SEQ ID NO. 32, SEQ ID NO. 34, SEQ ID NO. 36, SEQ ID NO. 38, SEQ ID NO. 40 or SEQ ID NO. 41.

A nucleic acid encoding the above protein, i.e. protein having the activity of the protein with the amino acid sequence of SEQ ID NO. 6, is also provided. That nucleic acid may have at least 70% sequence identity to SEQ ID NO. 5, SEQ ID NO. 7, SEQ ID NO. 9, SEQ ID NO. 11, SEQ ID NO. 13, SEQ ID NO. 15, SEQ ID NO. 17, SEQ ID NO. 19, SEQ ID NO. 21, SEQ ID NO. 23, SEQ ID NO. 25, SEQ ID NO. 27, SEQ ID NO. 29, SEQ ID NO. 31, SEQ ID NO. 33, SEQ ID NO. 35, SEQ ID NO. 37 and SEQ ID NO. 39.

### FIGURE LEGENDS

FIG. 1: HDC1 proteins have extended from ancestral Rxt3 proteins. (A) Cluster dendrogram of predicted protein sequences of HDC1/Rxt3 genes in yeast, algae, protozoa, mosses and higher plants, based on alignment of predicted amino acid sequences provided in Supplemental File 1. (B) Schematic view of conserved and novel parts of higher plant HDC1 proteins. For the Rxt3 part of the protein an alignment of the A. thaliana (At) sequence with sequences from Brachypodium distachyon (Bd) HDC1 and yeast (Sc) Rxt3 to A. thaliana (At) is inserted. A conserved Protein domain family signature ‘histone de-acetylation Rxt3’ (PF08642) is marked with a box.

FIG. 2: HDC1 is a ubiquitous nuclear protein. Tissue expression pattern and sub-cellular localization of HDC1. GUS staining shows HDC1 promoter activity in A. thaliana seeds (A), root and shoot of seedlings (B) and mature plants (C), rosette leaves (D) and flower buds (E). No staining is visible inside anthers or stigmas (F, arrows). Nuclear localization of GFP-HDC1 in epidermal leaf cells of transiently expressing N. tabacum (G) and in root cells of stably expressing A. thaliana plants (H, J). No GFP signal is seen inside the nucleolus (J, arrows). Scale bar in J is 50 μm.

FIG. 3: Co-localization of HDC1 with HDA6 and HDA19 within nuclei of transiently expressing tobacco epidermis cells. High-magnification images of nuclei in tobacco (N. benthamiana) epidermal leaf cells after transient expression of GFP-HDC1 and RFP-HDA6 or RFP-HDA19. Each row contains the following images from left to right: bright field, GFP fluorescence, RFP fluorescence, GFP/RFP overlay, quantitative comparison GFP and RFP signals along line scan (arrows in overlay images). HDC1 co-localizes with HDA6 (A-C) and HDA19 (D-F) in the entire nucleus (A, D), in distinct speckles (B-E) or in the nucleolus (C, F). Scale bar is 10 μm.

FIG. 4: HDC1 interacts with histone deacetylases HDA6 and HDA19 in a ratiometric BiFC assay. (A) ‘2-in-1’ vectors constructed for ratiometric BiFC assay containing N- and C-terminal halves of YFP (nYFP, cYFP) fused to HDC1, HDA6, HDA19 and SIN3 as well as a full-length RFP. (B) Signals of YFP (top row) and RFP (middle row) in nuclei of tobacco leaf cells after transient expression of nYFP-HDC1 with cYFP-HDA6, cYFP-HDA19 or cYFP-SIN3 (negative control). nYFP-SIN3 was also expressed with cYFP-HDA19 (positive control). The bottom row shows the bright field image. Scale bar is 10 μm. (C) YFP/RFP signal ratio in individual nuclei (means±SE, n≧20 cells from 3 independently transformed plants). Asterisks indicate significant differences (p<0.001) to the signal ratio obtained for HDC1-SIN3.

FIG. 5: HDC1 interacts with histone deacetylases in planta and facilitates H3K9/14 deacetylation. (A) Anti-His Western blots of recombinant HDC1-His after in-vitro pull-down with recombinant GST-HDA6 (second lane) and GST-HDA19 (third lane). The first lane contains a positive control (recombinant HDC1-His), the last lane contains a negative control (pull down with GST alone). (B) Anti-HDC1 Western blots of native HDC1 after pull-down from nuclei-enriched protein samples of A. thaliana wildtype (WT, left) or HDC1 knockout plants (hdc1-1, right) with recombinant GST-HDA6 (second lanes) or GST-HDA19 (third lanes). HDC1 is recognized in the untreated protein samples from wildtype (input), and in wildtype samples after pull-down with GST-HDA6/19 but not with GST alone. HDC1 is not found in protein samples (input or pull-downs) from knockout plants. The lower panel shows the membrane re-probed with anti-GST confirming presence of the bait. (C) Western blot with anti-H3K9K14ac shows increased amounts of acetylated H3K49K14 in protein extract from A. thaliana hdc1-1 plants compared to wildtype (left blot). After complementation (expression of HDC1 in hdc1-1, HDC1c) H3K49K14ac is reverted to wildtype level (right blot). Total H3 (loading control) was detected with anti(α)-H3. H3K49K14Ac/H3 signal ratios in wildtype, hdc1-1 and HDC1c lines were determined after quantification of bands with Image J. Bars are means±SE from at least three Western blots. Asterisk indicates significant (p<0.05) difference to WT and to HDC1c.

FIG. 6: Confirmation of hdc1-1 knockout and HDC1 over-expressing lines. A: Position of T-DNA and primer pairs in the genomic DNA of A. thaliana hdc1-1 knockout line (GABI-Kat 054G03). Numbers indicate position of primer pairs used for genotyping. B: HDC1 mRNA in wildtype and hdc1-1 as determined by semi-quantitative RT-PCR using the primer pairs indicated in A. Tubulin 9 (Tub 9) was used as a loading control. C: Western blot with anti-HDC1 detects HDC1 in A. thaliana wildtype but not in hdc1-1. Detection of the larger HDC1-GFP fusion protein transiently expressed in tobacco is shown for comparison. Rubisco (loading control) was detected by Ponceau staining. D: HDC1 mRNA levels (relative to Tub 9) in two lines overexpressing HDC1 under control of 35-S or Ubiquitin-10 promoters.

FIG. 7: Salk150126 and Sail1263E05 are not hdc1 knockouts. A: Position of T-DNA and primer pairs in the genomic DNA for Salk_{—}150126 and Sail_{—}1263_E05 lines. B: HDC1 mRNA levels in A. thaliana wildtype, Salk_{—}150126 and Sail_{—}1263_E05 using the primer pairs indicated in A. RpII is RNA polymeraseII (loading control). Asterisks indicate significant differences to the wild type (p<0.05). C: Germination rates of A. thaliana wildtype (black), Salk_{—}150126 (grey stripes) and Sail_{—}1263_E05 (light grey stripes) on agar containing different concentrations of ABA. Bars are means+/−SE of at least 3 plates containing at least 50 seeds each. Note that neither of the lines shows ABA hypersensitivity.

FIG. 8: HDC1 de-sensitizes seedlings to salt, mannitol, ABA and PAC. Germination rates of A. thaliana wildtype (black), hdc1-1 knockout (white) and HDC1 overexpressing (OX) lines (grey) on agar containing different concentrations of salt (NaCl, A), mannitol (B), ABA (C) or GA-biosynthesis inhibitor paclobutrazol (PAC, D). Germination rates in % reflect the number of seedlings that had developed cotyledons on day 6 after sowing, normalized to the total number of seeds sown. Bars are means±SE of at least 3 plates containing 50 seeds each. Asterisks indicate significant differences (p<0.05) to wildtype. A photo of the seedlings is shown in FIG. 9.

FIG. 9: A: Appearance of young A. thaliana seedlings on day 6 after sowing. Wildtype (upper third of plate), hdc1-1 (centre) and OX (lower) seeds were imbibed and allowed to germinate on half strength Murashige Skoog medium without (control) or with 0.3 added. Pictures were taken on the same day as germination rate was scored. Note that without ABA, number and size of seedlings is similar for all lines. B: Transcript levels for embryogenesis related genes ABI3, FUS3 and LEC1 in wildtype (WT, black), hdc1-1 knockout (KO, white) and HDC1 over-expressing (OX, grey) seedlings 2-6 days after germination (DAG). Bars represent means of 4 technical qPCR replicates with mRNA pooled from 50 seedlings. Asterisk indicates significant difference to wildtype (p<0.05).

FIG. 10: HDA6 over-expression does not affect germination or growth. A: Germination rates of imbibed A. thaliana wildtype (black), 35S::HDC1 (light grey) and 35S::HDA6 (dark grey) seeds. Germination rates in % reflect the number of seedlings that had developed cotyledons on day 6, normalized to the total number of seeds plated out. Bars are means±SE of 3 plates containing 50 seeds each. Asterisks indicate significant differences (p<0.05) to wildtype. B: Transcript levels of HDA6 in wildtype and 35S::HDA6 lines. C: Shoot weights (FW: fresh weight, DW: dry weight of 5-weeks old plants). Bars are means of 8 plants.

FIG. 11: Histone deacetylation is required for ABA-hyposensitivity. Germination rates of A. thaliana wildtype (B) and HDC1 overexpressing plants (B, C) on agar containing increasing concentrations of ABA with or without 0.3 or 3 μM histone de-acetylation inhibitor trichostatin A (TSA). Other details as in FIG. 8.

FIG. 12: Knockout of HDC1 delays flowering without altering the plastochron. (A) Plastochron of A. thaliana wildtype (black), hdc1-1 knockout (white) and HDC1 OX plants (grey) growing on soil in long-day conditions. Bars are means of 3 plants±SE. (B) Plant age at bolting. Bars are means±SE of 10-15 plants. (C) Number of leaves at bolting. Bars are means±SE of 10-15 plants. (D) FLC transcript levels on day 28. Bars are means±SE of 3 plants. Asterisks indicate differences to wildtype at p<0.05.

FIG. 13: HDC1 promotes vegetative plant growth. (A) Shoot and root fresh weight (FW) of A. thaliana wildtype (black), hdc1-1 knockout (white) and HDC1 OX plants dark (grey). Plants were grown hydroponically in short-day conditions. Bars show mean FW of 6 plants±SE. Asterisks indicate difference to wildtype at p<0.05. For determination of dry weights (DW) tissues of the 6 plants harvested on day 35 were pooled and dried. The combined weight was divided by the plant number. Appearance of the plants on day 35 is shown in the photo on the right. (B) Shoot weights of hdc1-1 knockout plants and of two independent complementation lines (35S::genomic HDC1 in hdc1-1 background). Bars are means of 5 plants±SE, each compared to the hdc1-1 plant grown in the same tray. The photo shows typical plant appearance (day 24, long-day conditions). Western blot of leaf protein extract with HDC1-antibody (aHDC1) reflects the amount of HDC1 protein in the plants. Ponceau stained Rubisco provides a loading control.

FIG. 14: HDC1 enhances leaf surface of expanding rosette leaves in young plants. Leaf surface areas of 2-weeks old A. thaliana wildtype (black), hdc1-1 (white)) and HDC1-OX (grey) plants grown on soil in long-day conditions. All plants had the same number of leaves (see FIG. 7A). Leaves were removed in order of appearance and analysed with Image J. Bars are means±SE of 3 plants. Asterisks indicate significant differences (p<0.05) to wildtype.

FIG. 15: HDA6 knockdown affects plant growth without delaying leaf development. A: Fresh and dry weights of 4-weeks old A. thaliana wildtype (Col-DR5, black) and hda6-knockdown (axe1-5, white dotted) plants. B: Leaf numbers in wildtype and axe1-5 mutants. Bars are means±SE of 5 plants.

FIG. 16: HDC1 Knockout/Overexpression deregulates salt-responsive genes. Transcript levels of salt-responsive genes in A. thaliana wild type (WT; black), hdc1-1 knockout (KO; white), and HDC1 overexpressing line (OX; gray). Plants were grown for 4 weeks in short-day conditions and subjected (+) or not (2) to 150 mM NaCl for 24 h in hydroponics. mRNA was pooled from three independently treated plant batches of five plants each. Each replicate treatment resulted in a significant increase of ABA (see FIG. 17). Transcript levels were normalized to those of tubulin 9 (TUB9). Bars are means of four technical qPCR replicates 6 SE. Asterisks indicate significant differences to the wild type (P<0.05). RAB18, RESPONSIVE TO ABA18.

FIG. 17: HDC1 has a small effect on ABA content after salt treatment. A: Shoot ABA content of wildtype (WT, black), hdc1-1 knockout (KO, white) and HDC1 over-expressing (OX, grey). Plants were grown for 4 weeks in short day conditions and subjected (+) or not (−) to 150 mM NaCl for 24 h in hydroponics. Absolute results from three independently treated plant batches are shown. B: Relative change of ABA content in hdc1-1 and HDC1-overexpressing plants compared to wildtype. ABA content was normalized to the ABA content of salt-treated wildtype plants in the same batch.

FIG. 18: HDC1 determines H3K9/K14 acetylation status of ABA1, DR4, PYL4 and RD29B. Relative amounts of DNA associated with acetylated H3K9/K14 for ABA1, DR4, PYL4 and RD29B as determined by ChIP-qPCR in A. thaliana wildtype (WT, black), hdc1-1 knockout (KO, white) and HDC1 over-expressing (OX, grey) plants. Leaf tissue was pooled from 4-weeks old plants grown in 3 independent batches 12 plants each. Chromatin extracted and immunoprecipitated with anti-H3K9K14Ac. qPCR-amplified ChIP-DNA was normalized to actin 2 and to input DNA (chromatin before immunoprecipitation). Bars are means of 4 technical qPCR-replicates±SE. Asterisks indicate significant differences to the wild type (p<0.05).

FIG. 19: HDC1 increases plant growth in well-watered and in water-limited conditions. (A) Rosette diameter and shoot weights (fresh weight; FW, dry weight: DW) of A. thaliana wildtype (black), hdc1-1 knockout (white) and HDC1 OX plants (grey). Plants were grown on soil in short-day conditions. The water-limited regime consisted in reducing water supply from day 14 to achieve a continuous relative soil water content of ˜50% of the control condition until the end of the experiment at day 40. Bars are means±SE of at least 24 plants. Asterisks indicate differences to wildtype at p<0.05. (B) Root and shoot weights of hydroponically grown plants growing in nutrient solution with 80 mM NaCl. Plant age at the beginning of the experiment was 29 days (short-day conditions). The first time point is 6 hours after salt application. Control plants grown in parallel without salt are shown in FIG. 8. Bars are mean fresh weights (FW)±SE of 6 plants per line. Asterisks indicate differences to wildtype at p<0.05. For determination of dry weight (DW) the tissues of 6 plants were pooled. Photos show plants of each line after 6 days in 80 mM NaCl.

FIG. 20: HDC1 increases biomass under control and drought conditions. Fresh weight per plant and per treatment of wheat wildtype (“Control”) and for 3 events (Event1, Event2 and Event3) performing better under drought as well as under control conditions. (Statistical significance: *=p<0.1, **=p<0.05).

FIG. 21: HDC1 increases number of heads. Number of heads per plant of wheat wildtype (“Control”) and for 2 events (Event4 and Event5) performing better under control conditions. (Statistical significance: *=p<0.1).

FIG. 22: HDC1 increases yield under control conditions. Yield in number of seeds per plant of wheat wildtype (“Control”) and for 2 events (Event4 and Event5) performing better under control conditions. (Statistical significance: **=p<0.05).

FIG. 23: HDC1 increases yield under control conditions. Yield in gram per plant of wheat wildtype (“Control”) and for 2 events (Event4 and Event5) performing better under control conditions.

FIG. 24: HDC1 has mRNA expression in transformed wheat plants. Event#1 and Event#2 clearly show mRNA expression. H stands for homozygous segregants, A stands for wild type segregants.

FIG. 25: HDC1 has mRNA expression in transformed wheat plants. Event#4 and Event#5 clearly show mRNA expression. H stands for homozygous segregants, A stands for wild type segregants.

### DETAILED DESCRIPTION

The present invention is based on the identification of a new HDAC-interacting protein that modulates plant ABA-sensitivity, growth and flowering, which is referred to as HISTONE DEACETYLASE COMPLEX 1 (HDC1). HDC1 is a single copy gene from Arabidopsis thaliana that is conserved in single or low copy number in other plant species including important crops. It has partial homology to the yeast gene Rxt3, a confirmed but functionally uncharacterised member of the LRpd3 complex (Carrozza et al., 2005, Bba-Gene Struct Expr 1731, 77-87; Chen et al., 2012, Curr Biol 22, 56-63). However, the function of HDC1 cannot be inferred from existing knowledge. Neither RXT3-type nor HDC1-type genes have been functionally characterized to date, and neither of them contain any known functional motifs. Furthermore, the plant genes are considerably longer than the ancestral RXT3 genes and could have acquired new functions. The inventors have shown that HDC1 is ubiquitously expressed in all diploid tissues and localizes to the nucleus where it interacts with histone deactelylases HDA6 and HDA19. HDC1 was found to promote histone de-acetylation as it appeared to be required for de-acetylation of lysine residues in histone 3. HDC1 overexpression resulted in three basic phenotypes (i) ABA-insensitivity of post-germination growth in seedlings and of stress-induced ABA-synthesis in mature plants, (ii) enhanced vegetative growth (biomass production) both in well-watered and in water-limited soils, and (iii) accelerated flowering, while in hdc1 knockout mutants these features were oppositely affected. A yield increase could also be observed in wheat plants. This shows that the phenotypes were indeed caused by HDC1, thereby identifying HDC1 as a critical determinant of plant growth, flowering and abiotic stress responses.

In accordance with a repressive function of histone deacetylation, it was found that transcript levels of several known stress-responsive genes were increased in hdc1-1 knockout plants and/or decreased in HDC1-OX plants. It is therefore thought that HDC1-facilitated histone deacetylation increases the amount of stimulus (e.g. ABA) and activator (e.g. transcription factor) required for de-repression of a gene upon stress thereby reducing its stress-sensitivity. Absence of HDC1 lowers the amount of stimulus required for de-repression but is not sufficient to activate transcription when stimulus and activator are absent (i.e. in control conditions). In the case of a stress-repressed gene, HDC1 decreases the efficiency of a given amount of constitutive activator thereby reducing transcript levels.

Without intending to limit the invention, it is therefore thought that HDC1 modulates ABA-sensitivity, growth and flowering by functioning as a universal scaffolding protein that enhances the apparent histone deacetylase activity by stabilizing the interaction of the enzymes with the substrate or with other regulatory proteins. Furthermore, contrary to over-expression of an HDA19 homolog in rice, which increased growth but also produced a range of developmental abnormalities (Zhou et al. 2005, supra), no such abnormalities occurred in HDC1-overexpressing plants. Hdc1 knockouts also did not reproduce aberrant developmental phenotypes observed in hda6/19 double mutants (Tian and Chen, 2001, Proc. Natl. Acad. Aci. USA 98, 200-205; Tanaka et al., 2008, Plant Physiol. 146, 149-161). Thus, indirect manipulation of histone deacetylase activity, via modulation of HDC1 expression levels as described herein, provides a means to effectively control plant growth and stress-sensitivity without developmental side effects.

Thus in a first embodiment, the invention provides a method for increasing the tolerance of a plant, plant part, plant organ or plant cell to stress conditions, preferably mild or moderate stress conditions; or for reducing ABA sensitivity of a plant, plant part, plant organ or plant cell; or for increasing biomass or yield or growth rate of a plant, plant organ or plant part; or for accelerating flowering time of a plant; comprising the step of increasing the functional expression (i.e. the expression and/or activity) of HDC1, i.e. a protein having the activity of the protein encoded by SEQ ID NO. 6, in said plant, plant part, plant organ or plant cell.

As used herein “a protein having the activity of the protein with the amino acid sequence of SEQ ID NO. 6” relates to any functional HDC1 protein. These include for example the plant HDC1 proteins as represented by SEQ ID NO. 6, SEQ ID NO. 8, SEQ ID NO. 10, SEQ ID NO. 12, SEQ ID NO. 14, SEQ ID NO. 16, SEQ ID NO. 18, SEQ ID NO. 20, SEQ ID NO. 22, SEQ ID NO. 24, SEQ ID NO. 26, SEQ ID NO. 28, SEQ ID NO. 30, SEQ ID NO. 32, SEQ ID NO. 34, SEQ ID NO. 36, SEQ ID NO. 38, SEQ ID NO. 40 and SEQ ID NO. 41, This also includes functional variants thereof, e.g. proteins having at least 50%, at least 60%, at least 70%, at least 75%, at least 80%, at least 85%, at least 90%, at least 95%, at least 96%, at least 97%, at least 98%, or at least 99% sequence identity to any of the amino acid sequences cited above that encode a functional HDC1 protein. Another example is based on the amino acid sequence enclosed by the nucleotide sequence of SEQ ID NO.: 42.

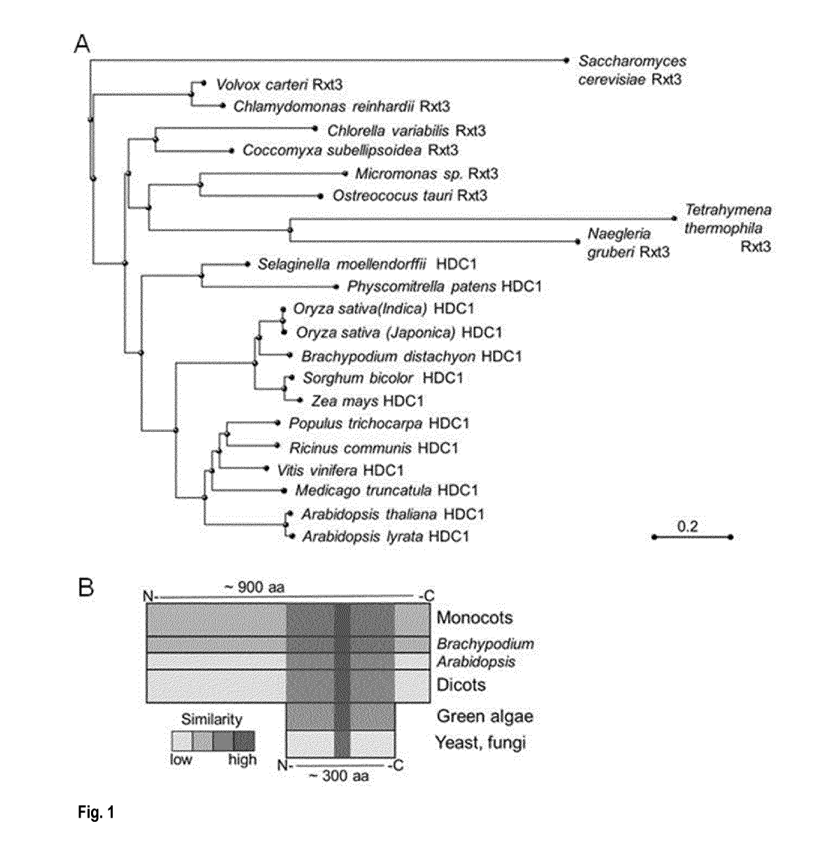

HDC1 proteins are ubiquitously expressed nuclear proteins of about 900 amino acids, of which the C-terminal half share sequence identity to the Rxt3-type proteins in green algae, protozoa and fungi (see FIG. 1), such as the 294-aa yeast protein Rxt3 (SEQ ID NO 4). HDC1 has furthermore been shown to be required for histone de-acetylation and to interact with various histone deacetylases (HDACs). Without intending to limit the invention to a particular mode of action, it is believed HDC1 functions as a relatively non-specific structural component to enhance the stability of histone deacetylation complexes, thereby increasing the efficiency of histone de-acetylation and downstream gene repression. HDC1 is not required for basal HDAC activity, as knockouts are viable, but thought to titrate the efficiency of HDACs. Further, as an enhancer of HDAC activity HDC1 depends on the catalytic function of HDACs but decreases sensitivity of processes that involve HDAC function to histone deacetylase inhibitor compounds (e.g. TSA) and to hormones such as ABA.

Increasing the expression and/or activity of an HDC1 protein can be achieved by modifying the endogenous gene or genes encoding such an HDC1 protein or by introducing a transgene, which when transcribed or expressed results in an increase of HDC1 protein expression and/or activity.

Thus, increasing the activity and or expression of HDC1 proteins in order to produce a plant or plant cell with increased tolerance to stress conditions or a plant with increased yield/biomass/growth or a plant with earlier flowering time can be achieved by providing that plant, or plant cell with a chimeric gene, which when expressed results in an increased activity and/or expression of a protein, e.g using the approaches as described above.

Unless indicated otherwise, the embodiments described below for the chimeric gene disclosed herein are also applicable to respective embodiments of other aspects disclosed herein.

In another embodiment, the invention provides a method for increasing the stress tolerance of a plant, plant part, plant organ or plant cell; or for increasing biomass or yield or growth of a plant, plant organ or plant part; or for accelerating flowering time of a plant, comprising the steps of expressing in said plant, plant part, plant organ or plant cell a chimeric gene comprising the following operably linked elements:
i. A plant-expressible promoter;
ii. A nucleic acid which when transcribed results in an increased activity and/or expression of a protein having the activity of the protein encoded by SEQ ID NO. 6; and
iii. A 3′ end region involved in transcription termination and polyadenylation functional in plants.

In one embodiment, a nucleic acid which when transcribed results in an increased activity and/or expression of a protein having the activity of the protein encoded by SEQ ID NO. 6 can encode an activating transcription factor that targets the promoter of the endogenous HDC1 gene present in the plant (e.g. the promoter such as represented by SEQ ID NO. 1), such that expression of the endogenous HDC1 gene is increased. Such transcription factors can be designed for example by coupling a non-specific transcription enhancer to a sequence specific DNA binding protein. Such techniques for designing transcription factors with a particular desired site specificity are e.g. described in Bogdanova and Voytas (2011, Science 333, p 1843-1846) and references therein.

In other embodiments, the nucleic acid can itself encode a HDC1 protein, thereby increasing the amount of functional HDC1 protein in the cell, such as proteins comprising the amino acid sequence of SEQ ID NO. 6, or functional variants thereof, e.g. proteins having at least 50%, at least 60%, at least 70%, at least 75%, at least 80%, at least 85%, at least 90%, at least 95%, at least 96%, at least 97%, at least 98%, or at least 99% sequence identity to any of the amino acid sequences cited above.

In a particular embodiment, the nucleic acid encodes an HDC1 protein and comprises the nucleotide sequence of SEQ ID NO. 5, SEQ ID NO. 7, SEQ ID NO. 9, SEQ ID NO. 11, SEQ ID NO. 13, SEQ ID NO. 15, SEQ ID NO. 17, SEQ ID NO. 19, SEQ ID NO. 21, SEQ ID NO. 23, SEQ ID NO. 25, SEQ ID NO. 27, SEQ ID NO. 29, SEQ ID NO. 31, SEQ ID NO. 33, SEQ ID NO. 35, SEQ ID NO. 37 and SEQ ID NO. 39, or variants thereof, e.g. nucleotide sequences having at least 50%, at least 60%, at least 70%, at least 75%, at least 80%, at least 85%, at least 90%, at least 95%, at least 96%, at least 97%, at least 98%, or at least 99% sequence identity to any of the nucleotide sequences cited above and which encode a functional HDC1 protein.

The “sequence identity” of two related nucleotide or amino acid sequences, expressed as a percentage, refers to the number of positions in the two optimally aligned sequences which have identical residues (×100) divided by the number of positions compared. A gap, i.e., a position in an alignment where a residue is present in one sequence but not in the other, is regarded as a position with non-identical residues. The “optimal alignment” of two sequences is found by aligning the two sequences over the entire length according to the Needleman and Wunsch global alignment algorithm (Needleman and Wunsch, 1970, J Mol Biol 48(3):443-53) in The European Molecular Biology Open Software Suite (EMBOSS, Rice et al., 2000, Trends in Genetics 16(6): 276-277; see e.g. http://www.ebi.ac.uk/emboss/align/index.html) using default settings (gap opening penalty=10 (for nucleotides)/10 (for proteins) and gap extension penalty=0.5 (for nucleotides)/0.5 (for proteins)). For nucleotides the default scoring matrix used is EDNAFULL and for proteins the default scoring matrix is EBLOSUM62.

Based on the available sequences, the skilled person can isolate genes encoding HDC1 other than the genes encoding protein with amino acid sequences or having the coding sequences mentioned above. Homologous nucleotide sequence may be identified and isolated by hybridization under stringent conditions using as probes identified nucleotide sequences.

“High stringency conditions” can be provided, for example, by hybridization at 65° C. in an aqueous solution containing 6×SSC (20×SSC contains 3.0 M NaCl, 0.3 M Na-citrate, pH 7.0), 5×Denhardt's (100×Denhardt's contains 2% Ficoll, 2% Polyvinyl pyrollidone, 2% Bovine Serum Albumin), 0.5% sodium dodecyl sulphate (SDS), and 20 μg/ml denaturated carrier DNA (single-stranded fish sperm DNA, with an average length of 120-3000 nucleotides) as non-specific competitor. Following hybridization, high stringency washing may be done in several steps, with a final wash (about 30 min) at the hybridization temperature in 0.2-0.1×SSC, 0.1% SDS.

“Moderate stringency conditions” refers to conditions equivalent to hybridization in the above described solution but at about 60-62° C. Moderate stringency washing may be done at the hybridization temperature in 1×SSC, 0.1% SDS.

“Low stringency” refers to conditions equivalent to hybridization in the above described solution at about 50-52° C. Low stringency washing may be done at the hybridization temperature in 2×SSC, 0.1% SDS. See also Sambrook et al. (1989) and Sambrook and Russell (2001).

Other sequences encoding HDC1 may also be obtained by DNA amplification using oligonucleotides specific for genes encoding HDC1 as primers, such as but not limited to oligonucleotides comprising or consisting of about 20 to about 50 consecutive nucleotides from the known nucleotide sequences or their complement.

A chimeric gene, as used herein, refers to a gene that is made up of heterologous elements that are operably linked to enable expression of the gene, whereby that combination is not normally found in nature. As such, the term “heterologous” refers to the relationship between two or more nucleic acid or protein sequences that are derived from different sources. For example, a promoter is heterologous with respect to an operably linked nucleic acid sequence, such as a coding sequence, if such a combination is not normally found in nature. In addition, a particular sequence may be “heterologous” with respect to a cell or organism into which it is inserted (i.e. does not naturally occur in that particular cell or organism).

The expression “operably linked” means that said elements of the chimeric gene are linked to one another in such a way that their function is coordinated and allows expression of the coding sequence, i.e. they are functionally linked. By way of example, a promoter is functionally linked to another nucleotide sequence when it is capable of ensuring transcription and ultimately expression of said other nucleotide sequence. Two proteins encoding nucleotide sequences, e.g. a transit peptide encoding nucleic acid sequence and a nucleic acid sequence encoding a protein having HDC1 activity, are functionally or operably linked to each other if they are connected in such a way that a fusion protein of first and second protein or polypeptide can be formed.

A gene, e.g. the chimeric gene of the invention, is said to be expressed when it leads to the formation of an expression product. An expression product denotes an intermediate or end product arising from the transcription and optionally translation of the nucleic acid, DNA or RNA, coding for such product, e.g. the second nucleic acid described herein. During the transcription process, a DNA sequence under control of regulatory regions, particularly the promoter, is transcribed into an RNA molecule. An RNA molecule may either itself form an expression product or be an intermediate product when it is capable of being translated into a peptide or protein. A gene is said to encode an RNA molecule as expression product when the RNA as the end product of the expression of the gene is, e.g., capable of interacting with another nucleic acid or protein. Examples of RNA expression products include inhibitory RNA such as e.g. sense RNA (co-suppression), antisense RNA, ribozymes, miRNA or siRNA, mRNA, rRNA and tRNA. A gene is said to encode a protein as expression product when the end product of the expression of the gene is a protein or peptide.

As the skilled person will be well aware, various promoters may be used to promote the transcription of the nucleic acid of the invention, i.e. the nucleic acid which when transcribed results in an increased activity and/or expression of an HDC1 protein. Such promoters include for example constitutive promoters, inducible promoters (e.g. stress-inducible promoters, drought-inducible promoters, hormone-inducible promoters, chemical-inducible promoters, etc.), tissue-specific promoters, developmentally regulated promoters and the like.

Thus, a plant expressible promoter can be a constitutive promoter, i.e. a promoter capable of directing high levels of expression in most cell types (in a spatio-temporal independent manner). Examples of plant expressible constitutive promoters include promoters of bacterial origin, such as the octopine synthase (OCS) and nopaline synthase (NOS) promoters from Agrobacterium, but also promoters of viral origin, such as that of the cauliflower mosaic virus (CaMV) 35S transcript (Hapster et al., 1988, Mol. Gen. Genet. 212: 182-190) or 19S RNAs genes (Odell et al., 1985, Nature. 6;313(6005):810-2; U.S. Pat. No. 5,352,605; WO 84/02913; Benfey et al., 1989, EMBO J. 8:2195-2202), the enhanced 2×35S promoter (Kay at al., 1987, Science 236:1299-1302; Datla et al. (1993), Plant Sci 94:139-149) promoters of the cassava vein mosaic virus (CsVMV; WO 97/48819, U.S. Pat. No. 7,053,205), 2×CsVMV (WO2004/053135) the circovirus (AU 689 311) promoter, the sugarcane bacilliform badnavirus (ScBV) promoter (Samac et al., 2004, Transgenic Res. 13(4):349-61), the figwort mosaic virus (FMV) promoter (Sanger et al., 1990, Plant Mol Biol. 14(3):433-43), the subterranean clover virus promoter No 4 or No 7 (WO 96/06932) and the enhanced 35S promoter as described in U.S. Pat. No. 5,164,316, U.S. Pat. No. 5,196,525, U.S. Pat. No. 5,322,938, U.S. Pat. No. 5,359,142 and U.S. Pat. No. 5,424,200. Among the promoters of plant origin, mention will be made of the promoters of the plant ribulose-biscarboxylase/oxygenase (Rubisco) small subunit promoter (U.S. Pat. No. 4,962,028; WO99/25842) from zea mays and sunflower, the promoter of the Arabidopsis thaliana histone H4 gene (Chabouté et al., 1987), the ubiquitin promoters (Holtorf et al., 1995, Plant Mol. Biol. 29:637-649, U.S. Pat. No. 5,510,474) of Maize, Rice and sugarcane, the Rice actin 1 promoter (Act-1, U.S. Pat. No. 5,641,876), the histone promoters as described in EP 0 507 698 A1, the Maize alcohol dehydrogenase 1 promoter (Adh-1) (from http://www.patentlens.net/daisy/promoters/242.html)). Also the small subunit promoter from Chrysanthemum may be used if that use is combined with the use of the respective terminator (Outchkourov et al., Planta, 216: 1003-1012, 2003).

A variety of plant gene promoters that regulate gene expression in response to environmental, hormonal, chemical, developmental signals, and in a tissue-active manner can be used for expression of a sequence in plants. Choice of a promoter is based largely on the phenotype of interest and is determined by such factors as tissue (e.g., seed, fruit, root, pollen, vascular tissue, flower, carpel, etc.), inducibility (e.g., in response to wounding, heat, cold, drought, light, pathogens, etc.), timing, developmental stage, and the like.

Additional promoters that can be used to practice this invention are those that elicit expression in response to stresses, such as the RD29 promoters that are activated in response to drought, low temperature, salt stress, or exposure to ABA (Yamaguchi-Shinozaki et al., 2004, Plant Cell, Vol. 6, 251-264; WO12/101118), but also promoters that are induced in response to heat (e.g., see Ainley et al. (1993) Plant Mol. Biol. 22: 13-23), light (e.g., the pea rbcS-3A promoter, Kuhlemeier et al. (1989) Plant Cell 1: 471-478, and the maize rbcS promoter, Schaffher and Sheen (1991) Plant Cell 3: 997-1012); wounding (e.g., wunl, Siebertz et al. (1989) Plant Cell 1: 961-968); pathogens (such as the PR-I promoter described in Buchel et al. (1999) Plant Mol. Biol. 40: 387-396, and the PDF 1.2 promoter described in Manners et al. (1998) Plant Mol. Biol. 38: 1071-1080), and chemicals such as methyl jasmonate or salicylic acid (e.g., see Gatz (1997) Annu. Rev. Plant Physiol. Plant Mol. Biol. 48: 89-108). In addition, the timing of the expression can be controlled by using promoters such as those acting at senescence (e.g., see Gan and Amasino (1995) Science 270: 1986-1988); or late seed development (e.g., see Odell et al. (1994) Plant Physiol. 106: 447-458).

Use may also be made of salt-inducible promoters such as the salt-inducible NHX1 promoter of rice landrace Pokkali (PKN) (Jahan et al., 6th International Rice Genetics symposium, 2009, poster abstract P4-37), the salt inducible promoter of the vacuolar H+-pyrophosphatase from Thellungiella halophila (TsVP1) (Sun et al., BMC Plant Biology 2010, 10:90), the salt-inducible promoter of the Citrus sinensis gene encoding phospholipid hydroperoxide isoform gpx1 (Avsian-Kretchmer et al., Plant Physiology July 2004 vol. 135, p 1685-1696).

In alternative embodiments, tissue-specific and/or developmental stage-specific promoters are used, e.g., promoter that can promote transcription only within a certain time frame of developmental stage within that tissue. See, e.g., Blazquez (1998) Plant Cell 10:791-800, characterizing the Arabidopsis LEAFY gene promoter. See also Cardon (1997) Plant J 12:367-77, describing the transcription factor SPL3, which recognizes a conserved sequence motif in the promoter region of the A. thaliana floral meristem identity gene API; and Mandel (1995) Plant Molecular Biology, Vol. 29, pp 995-1004, describing the meristem promoter eIF4. Tissue specific promoters which are active throughout the life cycle of a particular tissue can be used. In one aspect, the nucleic acids of the invention are operably linked to a promoter active primarily only in cotton fiber cells, in one aspect, the nucleic acids of the invention are operably linked to a promoter active primarily during the stages of cotton fiber cell elongation, e.g., as described by Rinehart (1996) supra. The nucleic acids can be operably linked to the FbI2A gene promoter to be preferentially expressed in cotton fiber cells (Ibid). See also, John (1997) Proc. Natl. Acad. Sci. USA 89:5769-5773; John, et al., U.S. Pat. Nos. 5,608,148 and 5,602,321, describing cotton fiber-specific promoters and methods for the construction of transgenic cotton plants. Root-specific promoters may also be used to express the nucleic acids of the invention. Examples of root-specific promoters include the promoter from the alcohol dehydrogenase gene (DeLisle (1990) Int. Rev. Cytol. 123:39-60) and promoters such as those disclosed in U.S. Pat. Nos. 5,618,988, 5,837,848 and 5,905,186. Other promoters that can be used to express the nucleic acids of the invention include, e.g., ovule-specific, embryo-specific, endosperm-specific, integument-specific, seed coat-specific promoters, or some combination thereof; a leaf-specific promoter (see, e.g., Busk (1997) Plant J. 11:1285 1295, describing a leaf-specific promoter in maize); the ORF 13 promoter from Agrobacterium rhizogenes (which exhibits high activity in roots, see, e.g., Hansen (1997) supra); a maize pollen specific promoter (see, e.g., Guerrero (1990) Mol. Gen. Genet. 224:161 168); a tomato promoter active during fruit ripening, senescence and abscission of leaves, a guard-cell preferential promoter e.g. as described in PCT/EP12/065608, and, to a lesser extent, of flowers can be used (see, e.g., Blume (1997) Plant J. 12:731 746); a pistil-specific promoter from the potato SK2 gene (see, e.g., Ficker (1997) Plant Mol. Biol. 35:425 431); the Blec4 gene from pea, which is active in epidermal tissue of vegetative and floral shoot apices of transgenic alfalfa making it a useful tool to target the expression of foreign genes to the epidermal layer of actively growing shoots or fibers; the ovule-specific BELI gene (see, e.g., Reiser (1995) Cell 83:735-742, GenBank No. U39944); and/or, the promoter in Klee, U.S. Pat. No. 5,589,583, describing a plant promoter region is capable of conferring high levels of transcription in meristematic tissue and/or rapidly dividing cells. Further tissue specific promoters that may be used according to the invention include: seed-specific promoters (such as the napin, phaseolin or DC3 promoter described in U.S. Pat. No. 5,773,697), fruit-specific promoters that are active during fruit ripening (such as the dru 1 promoter (U.S. Pat. No. 5,783,393), or the 2AI 1 promoter (e.g., see U.S. Pat. No. 4,943,674) and the tomato polygalacturonase promoter (e.g., see Bird et al. (1988) Plant Mol. Biol. 11: 651-662), flower-specific promoters (e.g., see Kaiser et al. (1995) Plant Mol. Biol. 28: 231-243), pollen-active promoters such as PTA29, PTA26 and PTAI 3 (e.g., see U.S. Pat. No. 5,792,929) and as described in e.g. Baerson et al. (1994 Plant Mol. Biol. 26: 1947-1959), promoters active in vascular tissue (e.g., see Ringli and Keller (1998) Plant Mol. Biol. 37: 977-988), carpels (e.g., see Ohl et al. (1990) Plant Cell 2:), pollen and ovules (e.g., see Baerson et al. (1993) Plant Mol. Biol. 22: 255-267). In alternative embodiments, plant promoters which are inducible upon exposure to plant hormones, such as auxins, are used to express the nucleic acids used to practice the invention. For example, the invention can use the auxin-response elements EI promoter fragment (AuxREs) in the soybean {Glycine max L.) (Liu (1997) Plant Physiol. 115:397-407); the auxin-responsive Arabidopsis GST6 promoter (also responsive to salicylic acid and hydrogen peroxide) (Chen (1996) Plant J. 10: 955-966); the auxin-inducible parC promoter from tobacco (Sakai (1996) 37:906-913); a plant biotin response element (Streit (1997) Mol. Plant Microbe Interact. 10:933-937); and, the promoter responsive to the stress hormone abscisic acid (ABA) (Sheen (1996) Science 274:1900-1902). Further hormone inducible promoters that may be used include auxin-inducible promoters (such as that described in van der Kop et al. (1999) Plant Mol. Biol. 39: 979-990 or Baumann et al., (1999) Plant Cell 11: 323-334), cytokinin-inducible promoter (e.g., see Guevara-Garcia (1998) Plant Mol. Biol. 38: 743-753), promoters responsive to gibberellin (e.g., see Shi et al. (1998) Plant Mol. Biol. 38: 1053-1060, Willmott et al. (1998) Plant Molec. Biol. 38: 817-825) and the like.

In alternative embodiments, nucleic acids used to practice the invention can also be operably linked to plant promoters which are inducible upon exposure to chemicals reagents which can be applied to the plant, such as herbicides or antibiotics. For example, the maize In2-2 promoter, activated by benzenesulfonamide herbicide safeners, can be used (De Veylder (1997) Plant Cell Physiol. 38:568-577); application of different herbicide safeners induces distinct gene expression patterns, including expression in the root, hydathodes, and the shoot apical meristem. Coding sequence can be under the control of, e.g., a tetracycline-inducible promoter, e.g., as described with transgenic tobacco plants containing the Avena sativa L. (oat) arginine decarboxylase gene (Masgrau (1997) Plant J. 11:465-473); or, a salicylic acid-responsive element (Stange (1997) Plant J. 11:1315-1324). Using chemically—{e.g., hormone- or pesticide-) induced promoters, i.e., promoter responsive to a chemical which can be applied to the transgenic plant in the field, expression of a polypeptide of the invention can be induced at a particular stage of development of the plant. Use may also be made of the estrogen-inducible expression system as described in U.S. Pat. No. 6,784,340 and Zuo et al. (2000, Plant J. 24: 265-273) to drive the expression of the nucleic acids used to practice the invention.

In alternative embodiments, the a promoter may be used whose host range is limited to target plant species, such as corn, rice, barley, wheat, potato or other crops, inducible at any stage of development of the crop.

In alternative embodiments, a tissue-specific plant promoter may drive expression of operably linked sequences in tissues other than the target tissue. In alternative embodiments, a tissue-specific promoter that drives expression preferentially in the target tissue or cell type, but may also lead to some expression in other tissues as well, is used.

In alternative embodiments, use may be made of promoter elements as e.g. described on http://arabidopsis.med.ohio-state.edu/AtcisDB/bindingsites.html., which in combination should result in a functional promoter.

According to the invention, use may also be made, in combination with the promoter, of other regulatory sequences, which are located between the promoter and the coding sequence, such as transcription activators (“enhancers”), for instance the translation activator of the tobacco mosaic virus (TMV) described in Application WO 87/07644, or of the tobacco etch virus (TEV) described by Carrington & Freed 1990, J. Virol. 64: 1590-1597, for example.

Other regulatory sequences that enhance the expression and/or activity of HDC1 may also be located within the chimeric gene. One example of such regulatory sequences are introns. Introns are intervening sequences present in the pre-mRNA but absent in the mature RNA following excision by a precise splicing mechanism. The ability of natural introns to enhance gene expression, a process referred to as intron-mediated enhancement (IME), has been known in various organisms, including mammals, insects, nematodes and plants (WO 07/098042, p 11-12). IME is generally described as a posttranscriptional mechanism leading to increased gene expression by stabilization of the transcript. The intron is required to be positioned between the promoter and the coding sequence in the normal orientation. However, some introns have also been described to affect translation, to function as promoters or as position and orientation independent transcriptional enhancers (Chaubet-Gigot et al., 2001, Plant Mol Biol. 45(1):17-30, p 27-28).

Examples of genes containing such introns include the 5′ introns from the rice actin 1 gene (see U.S. Pat. No. 5,641,876), the rice actin 2 gene, the maize sucrose synthase gene (Clancy and Hannah, 2002, Plant Physiol. 130(2):918-29), the maize alcohol dehydrogenase-1 (Adh-1) and Bronze-1 genes (Callis et al. 1987 Genes Dev. 1(10):1183-200; Mascarenhas et al. 1990, Plant Mol Biol. 15(6):913-20), the maize heat shock protein 70 gene (see U.S. Pat. No. 5,593,874), the maize shrunken 1 gene, the light sensitive 1 gene of Solanum tuberosum, and the heat shock protein 70 gene of Petunia hybrida (see U.S. Pat. No. 5,659,122), the replacement histone H3 gene from alfalfa (Keleman et al. 2002 Transgenic Res. 11(1):69-72) and either replacement histone H3 (histone H3.3-like) gene of Arabidopsis thaliana (Chaubet-Gigot et al., 2001, Plant Mol Biol. 45(1):17-30).

Other suitable regulatory sequences include 5′ UTRs. As used herein, a 5′UTR, also referred to as leader sequence, is a particular region of a messenger RNA (mRNA) located between the transcription start site and the start codon of the coding region. It is involved in mRNA stability and translation efficiency. For example, the 5′ untranslated leader of a petunia chlorophyll a/b binding protein gene downstream of the 35S transcription start site can be utilized to augment steady-state levels of reporter gene expression (Harpster et al., 1988, Mol Gen Genet. 212(1):182-90). WO95/006742 describes the use of 5′ non-translated leader sequences derived from genes coding for heat shock proteins to increase transgene expression.

The chimeric gene may also comprise a 3′ end region, i.e. a transcription termination or polyadenylation sequence, operable in plant cells. As a transcription termination or polyadenylation sequence, use may be made of any corresponding sequence of bacterial origin, such as for example the nos terminator of Agrobacterium tumefaciens, of viral origin, such as for example the CaMV 35S terminator, or of plant origin, such as for example a histone terminator as described in published Patent Application EP 0 633 317 A1. The polyadenylation region can be derived from the natural gene, from a variety of other plant genes, or from T-DNA. The 3′ end sequence to be added may be derived from, for example, the nopaline synthase or octopine synthase genes, or alternatively from another plant gene, or less preferably from any other eukaryotic gene.

Other measures to increase the expression that may be applied is optimizing the coding region for expression in the target organism, which may include adapting the codon usage, CG content, and elimination of unwanted nucleotide sequences (e.g. premature polyadenylation signals, cryptic intron splice sites, ATTTA pentamers, CCAAT box sequences, sequences that effect pre-mRNA splicing by secondary RNA structure formation such as long CG or AT stretches).

The coding sequence of the chimeric gene may further be modified as to increase protein stability, prevent protein degradation, enhance protein activity of the encoded HDC1 protein, for instance by introducing or deleting sites involved in post-translational modifications, such as sumoylation, ubiquitination, phosphorylation etc.

The HDC1 sequence as represented by SEQ ID NO. 6 contains a relatively high number of predicted sumoylation sites, suggesting that sumoylation plays an important role in maintaining HDC1 protein levels/activity. About 20% of lysines are concerned, compared to 7-14% in a random selection of proteins of similar length. The probability scores are extremely high (e.g. 94% for K273, K426, K192) and the sites are well conserved in HDC1 sequences of other plant species such as the HDC1 sequences described above. Sumoylation as a protective mechanism against degradation of HDC1 protein is supported by the finding that knockout of SUMO E3 ligase SIZ1 causes ABA-hypersensitivity and thus phenocopies hdc1 knockout plants (Miura et al., (2009) PNAS 13, 5418-5423). Miura et al. found that KO of a SUM01 ER ligase (SIZ1), which links the SUM01 protein to the sumoylated target proteins, causes ABA-sensitivity. This suggests that HDC1 function (whether resulting from expression of the endogenous gene or from an introduced transgene) can be further enhanced by overexpression of SUMO E3 ligases.

In order to further increase HDC1 functional expression, the nucleic acid of the chimeric gene encoding the HDC1 protein can be modified such that the encoded HDC1 protein interacts more tightly to HDAC proteins, for example by optimizing HDAC binding sites or introducing more HDAC binding sites.

In a further embodiment, increasing the functional expression (i.e. the expression and/or activity) of HDC1, i.e. a protein having the activity of the protein encoded by SEQ ID NO. 6, can be achieved by modifying the endogenous gene(s) encoding an HDC1 protein. This can be done through, for example, T-DNA activation tagging, mutagenesis (e.g. EMS mutagenesis) or by targeted genome engineering technologies. Using such technologies for example, the endogenous promoter can be modified such that it drives higher levels of expression, or the endogenous promoter can be replaced with a stronger promoter, or mutations can be introduced into the coding region that enhance mRNA stability, translation efficiency, protein activity and/or stability, similar to the above described methods for enhancing the expression of the introduced chimeric gene.

T-DNA activation tagging (Memelink, 2003, Methods Mol Biol. 236:345) is a method to activate endogenous genes by random insertion of a T-DNA carrying promoter or enhancer elements, which can cause transcriptional activation of flanking plant genes. The method can consist of generating a large number of transformed plants or plant cells using a specialized T-DNA construct, followed by selection for the desired phenotype.

Targeted genome engineering refers to generating intended and directed modifications into the genome. Such intended modifications can be insertions at specific genomic locations, deletions of specific endogenous sequences, and replacements of endogenous sequences. Targeted genome engineering can be based on homologous recombination. Targeted genome engineering to increase the functional expression of the HDC1 endogene can consist of insertion of a promoter, stronger than the endogenous promoter, in front of the HDC1 coding sequence, or insert an enhancer to increase promoter activity. Such techniques can also be applied e.g. to insert elements increasing RNA stability or enhancing translation of the encoded mRNA, or modify the coding sequence to enhance translation, protein stability and activity, similar to the above described methods for enhancing the expression of the introduced chimeric gene.

“Mutagenesis”, as used herein, refers to the process in which plant cells are subjected to a technique which induces mutations in the DNA of the cells, such as contact with a mutagenic agent, such as a chemical substance (such as ethylmethylsulfonate (EMS), ethylnitrosourea (ENU), etc.) or ionizing radiation (neutrons (such as in fast neutron mutagenesis, etc.), alpha rays, gamma rays (such as that supplied by a Cobalt 60 source), X-rays, UV-radiation, etc.), or targeted mutagenesis methods e.g. via oligonucleotides (e.g. KeyBase® technology). These methods can also be applied to modify the endogenous HDC1 encoding gene(s) as desired.

Expression of a transcript (e.g. an mRNA) of a protein can be measured according to various methods known in the art such as (quantitative) RT-PCR, northern blotting, microarray analysis, western blotting, ELISA and the like.

Increased expression, as used herein, refers to increase in expression level of at least 2%, or at least 5%, or at least 10%, or at least 15%, or at least 20%, or at least 25%, or at least 30%, or at least 40%, or at least 50% or even more. Said increase is an increase with respect to the expression in control plants.

Stress conditions, as used herein, refers e.g. to stress imposed by the application of chemical compounds (e.g., herbicides, fungicides, insecticides, plant growth regulators, adjuvants, fertilizers), exposure to abiotic stress (e.g., drought, waterlogging, submergence, high light conditions, high UV radiation, increased hydrogen peroxide levels, extreme (high or low) temperatures, ozone and other atmospheric pollutants, soil salinity or heavy metals, hypoxia, anoxia, osmotic stress, oxidative stress, low nutrient levels such as nitrogen or phosphor etc.) or biotic stress (e.g., pathogen or pest infection including infection by fungi, viruses, bacteria, insects, nematodes, mycoplasms and mycoplasma like organisms, etc.). Stress may also be imposed by hormones such as ABA or compound influencing hormone activity.

Drought, salinity, extreme temperatures, high light stress and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest.

Applying the teaching of the present invention, an increase in yield and/or growth rate occurs whether the plant is under non-stress conditions or whether the plant is exposed to various mild or moderate stress conditions compared to control plants. Plants typically respond to exposure to stress by growing more slowly. In conditions of severe stress or chronic stress, the plant may even stop growing altogether. The condition of moderate stress on the other hand is defined herein as being any stress to which a plant is exposed which does not result in the plant ceasing to grow altogether without the capacity to resume growth. Moderate stress in the sense of the invention leads to a reduction in the growth of the stressed plants of less than 40%, 35% or 30%, preferably less than 25%, 20% or 15%, more preferably less than 14%, 13%, 12%, 11% or 10% or less when compared to the control plant under non-stress conditions. Due to advances in agricultural practices (irrigation, fertilization, pesticide treatments) severe stresses are not often encountered in cultivated crop plants. As a consequence, the compromised growth induced by moderate stress is often an undesirable feature for agriculture, moderate stresses are the biotic and/or abiotic (environmental) stresses to which a plant is exposed under standard agricultural conditions. For example the stress as described in the Examples below are considered to constitute moderate or moderate stress conditions. The term “non-stress” conditions as used herein are those environmental conditions that allow optimal growth of plants.

In relation to the present invention, the effects on the plant of moderate stress can be compensated for by reducing the ABA sensitivity of a plant, as is the case when the activity and/or expression of the HDC1 protein is increased according to the present invention. Likewise, severe stress cannot be compensated for by reducing ABA sensitivity, and in such cases it may be preferred to decrease the activity and or expression of the HDC1 protein of the invention, as will be set forth further below.

A “control plant” as used herein is generally a plant of the same species which has wild-type levels of HDC1. “Wild-type levels of HDC1” as used herein refers to the typical levels of HDC1 protein in a plant as it most commonly occurs in nature. Said control plant has thus not been provided either with a nucleic acid molecule which when expressed increases the expression and/or activity of HDC1, nor has it been provided with a nucleic acid molecule which when expressed decreases the expression and/or activity of HDC1.

Various methods are available in the art to measure the tolerance of plants, plant parts, plant cells or seeds to various stresses, some of which are described in the examples here below. Increased stress tolerance will usually be apparent from the general appearance of the plants and may be measured e.g., by increased biomass production, continued vegetative growth under adverse conditions or higher seed yield. Stress tolerant plant have a broader growth spectrum, i.e. they are able to withstand a broader range of climatological and other abiotic changes, without yield penalty, as compared to control plants. Biochemically, stress tolerance may be apparent as the higher NAD+−NADH/ATP content and lower production of reactive oxygen species of stress tolerant plants compared to control plants under stress condition. Stress tolerance may also be apparent as the higher chlorophyll content, higher germination rates, higher photosynthesis and lower chlorophyll fluorescence under stress conditions in stress tolerant plants compared to control plants under the same conditions.

It will be clear that it is also not required that the plant be grown continuously under the adverse conditions for the stress tolerance to become apparent. Usually, the difference in stress tolerance between a plant or plant cell produced according to the invention and a control plant or plant cell will become apparent even when only a relatively short period of adverse conditions is encountered during growth.

Yield or biomass, as used herein, refers to seed number/weight, fruit number/weight, fresh weight, dry weight, leaf number/area, plant height, branching, boll number/size, fiber length, seed oil content, seed protein content, seed carbohydrate content. An increased growth rate as used herein refers to a period of increased growth or allocation to one or more of these cells or tissues that comprise the aforementioned plant organs.

An increase in biomass or yield or growth can be an increase of at least 2%, or at least 5%, or at least 10%, or at least 15%, or at least 20%, or at least 25%, or at least 30%, or at least 40%, or at least 50%. Said increase is an increase with respect to biomass or yield or growth of control plants.

Abscisic acid (ABA) is a phytohormone which functions in many plant developmental processes, including seed dormancy. Furthermore, ABA mediates stress responses in plants in reaction to water stress, high-salt stress, cold stress (Mansfield 1987, p. 411-430. In: P. J. Davies (ed.). Plant hormones and their role in plant growth and development. Martinus Nijhoff Publishers, Dordrecht; Yamaguchi-Shinozaki 1993, Plant Physiol. 101, 1119-1120; Yamaguchi-Shinozaki 1994, Plant Cell 6, 251-264) and plant pathogens Seo and Koshiba, 2002, Trends Plant Sci. 7, 41-48). ABA is a sesquiterpenoid (15-carbon) which is partially produced via the mevalonic pathway in chloroplasts and other plastids. It is synthesized partially in the chloroplasts and accordingly, biosynthesis primarily occurs in the leaves. The production of ABA is increased by stresses such as water loss and freezing temperatures. It is believed that biosynthesis occurs indirectly through the production of carotenoids. Physiological responses known to be associated with abscisic acid include stimulation of the closure of stomata, inhibition of seedling or shoot growth, induction of storage protein synthesis in seeds and inhibition of the effect of gibberellins on stimulating de novo synthesis of α-amylase. Basic ABA levels may differ considerably from plant to plant. For example, the basal concentration of ABA in non-stressed Arabidopsis leaves is 2 to 3 ng/g fresh weight (Lopez-Carbonell and Jauregui, 2005). Under water-stress conditions, the ABA concentration reaches 10 to 21 ng/g fresh weight.

ABA sensitivity can be measured e.g. as described herein below. ABA sensitivity can also be measured by measurement of stomatal aperture (Zhang et al. 2009, EurAsia J BioSci 3, 10-16), measurement of ion current s (Armstrong et al 1995, PNAS 92:9520-4; Marten et al. 2007, Plant Physiol. Vol. 143, 28037) or measurement of ABA-dependent gene expression by microarrays, RNA-sequencing, RT-PCR or RNA gel blotting (Hoth et al. 2002, Journal of Cell Science 115, 4891-4900).

Decrease in ABA sensitivity can be a decrease of at least 2%, or at least 5%, or at least 10%, or at least 15%, or at least 20%, or at least 25%, or at least 30%, or at least 40%, or at least 50%. Said decrease is a decrease with respect to ABA sensitivity of control plants.

Thus, a plant made according to the invention having an increased HDC1 expression and/or activity can have at least one of the following phenotypes when compared to control plants, especially under adverse conditions, such as water limiting conditions, including but not limited to: increased overall plant yield, increased root mass, increased root length, increased leaf size, increased ear size, increased seed size, increased endosperm size, improved standability, alterations in the relative size of embryos and endosperms leading to changes in the relative levels of protein, oil and/or starch in the seeds, altered floral development, changes in leaf number, altered leaf surface, altered vasculature, altered internodes, alterations in leaf senescence, absence of tassels, absence of functional pollen bearing tassels, or increased plant size when compared to a non-modified plant under normal growth conditions or under adverse conditions, such as water limiting conditions.

In certain embodiments, the invention provides methods for enhancing survival of a plant, plant part, plant organ or plant cell under severe stress conditions, methods for enhancing recovery after severe stress of a plant, plant part, plant organ or plant cell, or methods for delaying the flowering time of a plant, comprising the step of decreasing the functional expression (expression and/or activity) protein having the activity of the protein encoded by SEQ ID NO. 6 (an HDC1 protein) in the plant, plant part, plant organ or plant cell.

It has been shown that after a period of severe drought stress (9 days), ABA-hypersensitive plants show an improved recovery when compared to wildtype plants (Tran et al., 2004, Plant Cell 16, 2481-2498, incorporated herein by reference). As it has presently been demonstrated that HDC1 downregulation (e.g. knockout) increases ABA sensitivity, it is believed that HDC1 downregulation under severe stress, by increasing ABA sensitivity, can enhance plant survival/recovery. Preferably, HDC1 downregulation is inducible, as plants with constitutive low levels of HDC1 and concomitant ABA hypersensitivity are thought to have a growth penalty under control conditions.

Reduce or eliminate the activity of HDC1 in a plant or plant cell can e.g be achieved by introducing a nucleic acid into the plant or plant cell that may inhibit the expression or function of the HDC1 polypeptide directly, by preventing transcription or translation of an HDC1 messenger RNA, or indirectly, by encoding a polypeptide that inhibits the transcription or translation of an HDC1 gene encoding a HDC1 polypeptide. Such nucleic acids are said to encode HDC1-inhibitory RNA molecules. Methods for inhibiting or eliminating the expression of a gene in a plant are well known in the art, and any such method may be used in the present invention to inhibit the expression of the HDC1 polypeptide. In other embodiments, a nucleic acid that encodes a polypeptide that inhibits the activity of an HDC1 polypeptide is introduced into a plant or plant cell. Many methods may be used to reduce or eliminate the activity of a HDC1 polypeptide.

In accordance with the present invention, the expression of HDC1 is inhibited if the transcript or protein level is statistically lower than the transcript or protein level of HDC1 in a plant that has not been modified to inhibit the expression of that HDC1. In particular embodiments of the invention, the transcript or protein level of the HCD1 may be less than 50%, less than 40%, less than 30%, less than 20%, less than 10%, or less than 5% of the mRNA or protein level of the same HDC1 in a plant that is not a mutant or that has not been modified to inhibit the expression of that HDC1.

In some embodiments of the present invention, a nucleic acid is introduced into a plant or plant cell that upon induction of expression, inhibits the expression of HDC1 in the plant or plant cell. Examples of nucleic acids that inhibit the expression of an HDC1 polypeptide are given below.

In some embodiments of the invention, inhibition of the expression of an HDC1 polypeptide may be obtained by sense suppression or cosuppression. For cosuppression, a chimeric gene or expression cassette is designed to express an RNA molecule corresponding to all or part of a messenger RNA encoding an HDC1 polypeptide in the “sense” orientation. The nucleic acid used for cosuppression may correspond to all or part of the sequence encoding the HDC1 polypeptide, all or part of the 5′ and/or 3′ untranslated region of an HDC1 polypeptide transcript or all or part of both the coding sequence and the untranslated regions of a transcript encoding an HDC1 polypeptide. A nucleic acid used for cosuppression or other gene silencing methods may share 99%, 98%, 97%, 96%, 95%, 94%, 93%, 92%, 91%, 90%, 89%, 88%, 87%, 85%, 80%, or less sequence identity with the target sequence. When portions of the nucleic acids (e.g., SEQ ID NO. 5, SEQ ID NO. 7, SEQ ID NO. 9, SEQ ID NO. 11, SEQ ID NO. 13, SEQ ID NO. 15, SEQ ID NO. 17, SEQ ID NO. 19, SEQ ID NO. 21, SEQ ID NO. 23, SEQ ID NO. 25, SEQ ID NO. 27, SEQ ID NO. 29, SEQ ID NO. 31, SEQ ID NO. 33, SEQ ID NO. 35, SEQ ID NO. 37 or SEQ ID NO. 39) are used to disrupt the expression of the target gene, generally, sequences of at least 15, 19, 20, 21, 22, 23, 24, 25, 26, 27, 28, 29, 30, 35, 40, 45, 50, 60, 70, 80, 90, 100, 200, 300, 400, 450, 500, 550, 600, 650, 700, 750, 800, 900, or 1000 contiguous nucleotides or greater may be used. In some embodiments where the nucleic acid comprises all or part of the coding region for the HDC1 polypeptide, the chimeric gene is designed to eliminate the start codon of the polynucleotide so that no protein product will be translated. Multiple plant lines transformed with the cosuppression chimeric gene can then be screened to identify those that show the desired (inducible) inhibition of HDC1 polypeptide expression.

In some embodiments of the invention, inhibition of the expression of the HDC1 polypeptide may be obtained by antisense suppression. For antisense suppression, the chimeric gene or expression cassette is designed to express an RNA molecule complementary to all or part of a messenger RNA encoding the HDC1 polypeptide. Overexpression of the antisense RNA molecule can result in reduced expression of the native gene. The polynucleotide for use in antisense suppression may correspond to all or part of the complement of the sequence encoding the HDC1 polypeptide, all or part of the complement of the 5′ and/or 3′ untranslated region of the HDC1 transcript or all or part of the complement of both the coding sequence and the untranslated regions of a transcript encoding the HDC1 polypeptide. In addition, the antisense nucleic acid may be fully complementary (i.e. 100% identical to the complement of the target sequence) or partially complementary (i.e. less than 100%, including but not limited to, 99%, 98%, 97%, 96%, 95%, 94%, 93%, 92%, 91%, 90%, 89%, 88%, 87%, 85%, 80%, identical to the complement of the target sequence, which in some embodiments is SEQ ID NO. 5, SEQ ID NO. 7, SEQ ID NO. 9, SEQ ID NO. 11, SEQ ID NO. 13, SEQ ID NO. 15, SEQ ID NO. 17, SEQ ID NO. 19, SEQ ID NO. 21, SEQ ID NO. 23, SEQ ID NO. 25, SEQ ID NO. 27, SEQ ID NO. 29, SEQ ID NO. 31, SEQ ID NO. 33, SEQ ID NO. 35, SEQ ID NO. 37 or SEQ ID NO. 39) to the target sequence. Furthermore, portions of the antisense nucleotides may be used to disrupt the expression of the target gene. Generally, sequences of at least 50 nucleotides, 100 nucleotides, 200 nucleotides, 300, 400, 450, 500, 550 or greater may be used. Multiple plant lines transformed with the antisense chimeric gene can then be screened to identify those that show the desired (inducible) inhibition of HDC1 polypeptide expression Methods for using antisense suppression to inhibit the expression of endogenous genes in plants are described, for example, in U.S. Pat. No. 5,759,829, which is herein incorporated by reference.

In some embodiments of the invention, inhibition of the expression of an HDC1 polypeptide may be obtained by double-stranded RNA (dsRNA) interference. For dsRNA interference, a sense RNA molecule like that described above for cosuppression and an antisense RNA molecule that is fully or partially complementary to the sense RNA molecule are expressed in the same cell, resulting in inhibition of the expression of the corresponding endogenous messenger RNA. Expression of the sense and antisense molecules can be accomplished by designing the chimeric gene to comprise both a sense sequence and an antisense sequence. Alternatively, separate chimeric genes may be used for the sense and antisense sequences. Multiple plant lines transformed with the dsRNA interference chimeric gene or chimeric genes are then screened to identify plant lines that show the desired (inducible) inhibition of HDC1 polypeptide expression. Methods for using dsRNA interference to inhibit the expression of endogenous plant genes are described in WO9949029, WO9953050, WO9961631 and WO0049035, each of which is herein incorporated by reference.

In some embodiments of the invention, inhibition of the expression of an HDC1 polypeptide may be obtained by hairpin RNA (hpRNA) interference or intron-containing hairpin RNA (ihpRNA) interference. These methods are highly efficient at inhibiting the expression of endogenous genes. See, Waterhouse and Helliwell, (2003) Nat. Rev. Genet. 4:29-38 and the references cited therein. For hpRNA interference, the chimeric gene is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene whose expression is to be inhibited, and an antisense sequence that is fully or partially complementary to the sense sequence. The antisense sequence may be located “upstream” of the sense sequence (i.e. the antisense sequence may be closer to the promoter driving expression of the hairpin RNA than the sense sequence). The base-paired stem region may correspond to a portion of a promoter sequence controlling expression of the gene to be inhibited. A nucleic acid designed to express an RNA molecule having a hairpin structure comprises a first nucleotide sequence and a second nucleotide sequence that is the complement of the first nucleotide sequence, and wherein the second nucleotide sequence is in an inverted orientation relative to the first nucleotide sequence. Thus, the base-paired stem region of the molecule generally determines the specificity of the RNA interference. The sense sequence and the antisense sequence are generally of similar lengths but may differ in length. Thus, these sequences may be portions or fragments of at least 10, 19, 20, 21, 22, 23, 24, 25, 26, 27, 28, 29, 30, 50, 70, 90, 100, 120, 140, 160, 180, 200, 220, 240, 260, 280, 300, 320, 340, 360, 380, 400, 500, 600, 700, 800, 900 nucleotides in length, or at least 1, 2, 3, 4, 5, 6, 7, 8, 9, or 10 kb in length. The loop region of the chimeric gene may vary in length. Thus, the loop region may be at least 10, 20, 30, 40, 50, 75, 100, 200, 300, 400, 500, 600, 700, 800, 900 nucleotides in length, or at least 1, 2, 3, 4, 5, 6, 7, 8, 9, or 10 kb in length. hpRNA molecules are highly efficient at inhibiting the expression of endogenous genes and the RNA interference they induce is inherited by subsequent generations of plants. See, for example, Waterhouse and Helliwell, (2003) Nat. Rev. Genet. 4:29-38. A transient assay for the efficiency of hpRNA constructs to silence gene expression in vivo has been described by Panstruga, et al. (2003) Mol. Biol. Rep. 30: 135-140, herein incorporated by reference. For ihpRNA, the interfering molecules have the same general structure as for hpRNA, but the RNA molecule additionally comprises an intron in the loop of the hairpin that is capable of being spliced in the cell in which the ihpRNA is expressed. The use of an intron minimizes the size of the loop in the hairpin RNA molecule following splicing, and this increases the efficiency of interference. See, for example, Smith et al (2000) Nature 407:319-320. In fact, Smith et al, show 100% suppression of endogenous gene expression using ihpRNA-mediated interference. In some embodiments, the intron is the ADHI intron 1. Methods for using ihpRNA interference to inhibit the expression of endogenous plant genes are described, for example, in Smith et al, (2000) Nature 407:319-320; Waterhouse and Helliwell, (2003) Nat. Rev. Genet. 4:29-38; Helliwell and Waterhouse, (2003) Methods 30:289-295 and US2003180945, each of which is herein incorporated by reference.

The chimeric gene for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, WO0200904 herein incorporated by reference.

Amplicon chimeric genes comprise a plant virus-derived sequence that contains all or part of the target gene but generally not all of the genes of the native virus. The viral sequences present in the transcription product of the chimeric gene allow the transcription product to direct its own replication. The transcripts produced by the amplicon may be either sense or antisense relative to the target sequence (i.e., the messenger RNA for the HDC1 polypeptide). Methods of using amplicons to inhibit the expression of endogenous plant genes are described, for example, in U.S. Pat. No. 6,635,805, which is herein incorporated by reference.

In some embodiments, the nucleic acid expressed by the chimeric gene of the invention is catalytic RNA or has ribozyme activity specific for the messenger RNA of the HDC1 polypeptide. Thus, the polynucleotide causes the degradation of the endogenous messenger RNA, resulting in reduced expression of the HDC1 polypeptide. This method is described, for example, in U.S. Pat. No. 4,987,071, herein incorporated by reference.

In some embodiments of the invention, inhibition of the expression of a HDC1 polypeptide may be obtained by RNA interference by expression of a nucleic acid encoding a micro RNA (miRNA). miRNAs are regulatory agents consisting of about 22 ribonucleotides. miRNA are highly efficient at inhibiting the expression of endogenous genes. See, for example Javier et al (2003) Nature 425:257-263, herein incorporated by reference. For miRNA interference, the chimeric gene is designed to express an RNA molecule that is modeled on an endogenous pre-miRNA gene wherein the endogenous miRNA and miRNA* sequence are replaced by sequences targeting the HDC1 mRNA. The miRNA gene encodes an RNA that forms a hairpin structure containing a 18-22-nucleotide, e.g. 21 nucleotide, sequence that is complementary to another endogenous gene (target sequence). For suppression of the HDC1, the 18-22-nucleotide sequence is selected from the target transcript sequence and contains 18-22 nucleotides of said target sequence in sense orientation (the miRNA* sequence) and a corresponding antisense sequence that is complementary to the sense sequence and complementary to the target mRNA (the miRNA sequence). No perfect complementarity between the miRNA and its target is required, but some mismatches are allowed. Up to 4 mismatches between the miRNA and miRNA* sequence are also allowed. miRNA molecules are highly efficient at inhibiting the expression of endogenous genes, and the RNA interference they induce is inherited by subsequent generations of plants.

In one embodiment, the nucleic acid encodes a zinc finger protein that binds to a gene encoding an HDC1 polypeptide, resulting in reduced expression of the gene. In particular embodiments, the zinc finger protein binds to a regulatory region of an HDC1 gene. In other embodiments, the zinc finger protein binds to a messenger RNA encoding an HDC1 polypeptide and prevents its translation. Methods of selecting sites for targeting by zinc finger proteins have been described, for example, in U.S. Pat. No. 6,453,242, and methods for using zinc finger proteins to inhibit the expression of genes in plants are described, for example, in US2003/0037355, each of which is herein incorporated by reference.

In another embodiment, the nucleic acid encoded a TALE protein that binds to a gene encoding aHDC1 polypeptide, resulting in reduced expression of the gene. In particular embodiments, the TALE protein binds to a regulatory region of an HDC1 gene. In other embodiments, the TALE protein binds to a messenger RNA encoding an HDC1 polypeptide and prevents its translation. Methods of selecting sites for targeting by TALE proteins have been described in e.g. Moscou M J, Bogdanove A J (2009) (A simple cipher governs DNA recognition by TAL effectors. Science 326:1501) and Morbizer R, Romer P, Boch J, Lahaye T (2010) (Regulation of selected genome loci using de novo-engineered transcription activator-like effector (TALE)-type transcription factors. Proc Natl Acad Sci USA 107:21617-21622).

In some embodiments, polypeptides or nucleic acids encoding polypeptides can be introduced into a plant, wherein the encoded polypeptide is capable of inhibiting the functional expression or activity of an HDC1 polypeptide.

In one embodiment, proteins or polypeptides capable of inhibiting the functional expression or activity of an HDC1 polypeptide include e.g. a nucleic acid encoding an antibody (or nanobody etc) that binds to an HDC1 polypeptide and reduces the activity thereof. In another embodiment, the binding of the antibody results in increased turnover of the antibody-HDC1 complex by cellular quality control mechanisms. The expression of antibodies in plant cells and the inhibition of molecular pathways by expression and binding of antibodies to proteins in plant cells are well known in the art. See, for example, Conrad and Sonnewald, (2003) Nature Biotech. 21:35-36, incorporated herein by reference.

In another embodiment, proteins capable of inhibiting the functional expression or activity of an HDC1 polypeptide may also be a dominant negative HDC1 protein or protein fragments. Dominant negative HDC1 proteins could for example be HDC1 proteins wherein HDAC binding sites have been modified, e.g. removed, thereby inhibiting HDAC function.

In an alternative embodiment, the plant or plant cell can be contacted with molecules interfering with HDC1 function by triggering aggregation of the target protein (interferor peptides) as e.g. described in WO2007/071789 and WO2008/148751.

In an even further embodiment, the plant or plant cell can be contacted with so-called alphabodies specific for HDC1, i.e. non-natural proteinaceous molecules that can antagonize protein function, as e.g. described in WO2009/030780, WO2010/066740 and WO2012/092970.

As a reduction of HDC1 function under non-stress or mild or moderate stress conditions is generally unfavourable, it will be understood that in the above methods, the reduction of the expression and/or activity of HDC1 is preferably inducible in/by the conditions under which it is desirable to reduce HDC1 expression and/or functions, such as severe stress conditions. As the person skilled in the art would readily understand, inducible expression of the above described nucleic acids expressed in the plant or plant cell that that result in an inhibition of the expression and/or activity of HDC1 in the plant or plant cell is operably linked to an inducible promoter. A list of inducible promoters is described in detail above.

In alternative embodiments, HDC1 downregulation can be induced at the desired moment using a spray (systemic application) with inhibitory nucleic acids, such as RNA or DNA molecules that function in RNA-mediated gene silencing (similar to the above described molecules) which target endogenous HDC1, as e.g. described in WO2011/112570 (incorporated herein by reference).

In further embodiments, the invention provides chimeric genes comprising a nucleic acid which when transcribed results in an increased or decreased activity and/or expression of HDC1, as described in detail above. Chimeric genes or vectors comprising the chimeric genes are also included in the invention.

Nucleic acids and chimeric genes used to practice the invention can be expressed by introduction into a plant cell by any means. For example, nucleic acids or expression constructs can be introduced into the genome of a desired plant host, or, the nucleic acids or chimeric genes can be episomes. Introduction into the genome of a desired plant can also be such that the host's HDC1 protein production is regulated by endogenous transcriptional or translational control elements, or by a heterologous promoter, e.g., a promoter of this invention.

“Introducing” in connection with the present application relates to the placing of genetic information in a plant cell or plant by artificial means, such as transformation. This can be effected by any method known in the art for introducing RNA or DNA into plant cells, tissues, protoplasts or whole plants. In addition to artificial introduction as described above, “introducing” also comprises introgressing genes as defined further below.

Transformation means introducing a nucleotide sequence into a plant in a manner to cause stable or transient expression of the sequence. Transformation and regeneration of both monocotyledonous and dicotyledonous plant cells is now routine, and the selection of the most appropriate transformation technique will be determined by the practitioner. The choice of method will vary with the type of plant to be transformed; those skilled in the art will recognize the suitability of particular methods for given plant types. Suitable methods can include, but are not limited to: electroporation of plant protoplasts; liposome-mediated transformation; polyethylene glycol (PEG) mediated transformation; transformation using viruses; micro-injection of plant cells; micro-projectile bombardment of plant cells; vacuum infiltration; and Agrobacterium-mediated transformation.

In alternative embodiments, the invention uses Agrobacterium tumefaciens mediated transformation. Also other bacteria capable of transferring nucleic acid molecules into plant cells may be used, such as certain soil bacteria of the order of the Rhizobiales, e.g. Rhizobiaceae (e.g. Rhizobium spp., Sinorhizobium spp., Agrobacterium spp); Phyllobacteriaceae (e.g. Mesorhizobium spp., Phyllobacterium spp.); Brucellaceae (e.g. Ochrobactrum spp.); Bradyrhizobiaceae (e.g. Bradyrhizobium spp.), and Xanthobacteraceae (e.g. Azorhizobium spp.), Agrobacterium spp., Rhizobium spp., Sinorhizobium spp., Mesorhizobium spp., Phyllobacterium spp. Ochrobactrum spp. and Bradyrhizobium spp., examples of which include Ochrobactrum sp., Rhizobium sp., Mesorhizobium loti, Sinorhizobium meliloti. Examples of Rhizobia include R. leguminosarum by, trifolii, R. leguminosarum bv, phaseoli and Rhizobium leguminosarum, by, viciae (U.S. Pat. No. 7,888,552). Other bacteria that can be employed to carry out the invention which are capable of transforming plants cells and induce the incorporation of foreign DNA into the plant genome are bacteria of the genera Azobacter (aerobic), Closterium (strictly anaerobic), Klebsiella (optionally aerobic), and Rhodospirillum (anaerobic, photosynthetically active). Transfer of a Ti plasmid was also found to confer tumor inducing ability on several Rhizobiaceae members such as Rhizobium trifolii, Rhizobium leguminosarum and Phyllobacterium myrsinacearum, while Rhizobium sp. NGR234, Sinorhizobium meliloti and Mesorhizobium loti could indeed be modified to mediate gene transfer to a number of diverse plants (Broothaerts et al., 2005, Nature, 433:629-633).

In alternative embodiments, making transgenic plants or seeds comprises incorporating sequences used to practice the invention and, in one aspect (optionally), marker genes into a target expression construct (e.g., a plasmid), along with positioning of the promoter and the terminator sequences. This can involve transferring the modified gene into the plant through a suitable method. For example, a construct may be introduced directly into the genomic DNA of the plant cell using techniques such as electroporation and microinjection of plant cell protoplasts, or the constructs can be introduced directly to plant tissue using ballistic methods, such as DNA particle bombardment. For example, see, e.g., Christou (1997) Plant Mol. Biol. 35:197-203; Pawlowski (1996) Mol. Biotechnol. 6:17-30; Klein (1987) Nature 327:70-73; Takumi (1997) Genes Genet. Syst. 72:63-69, discussing use of particle bombardment to introduce transgenes into wheat; and Adam (1997) supra, for use of particle bombardment to introduce YACs into plant cells. For example, Rinehart (1997) supra, used particle bombardment to generate transgenic cotton plants. Apparatus for accelerating particles is described U.S. Pat. No. 5,015,580; and, the commercially available BioRad (Biolistics) PDS-2000 particle acceleration instrument; see also, John, U.S. Pat. No. 5,608,148; and Ellis, U.S. Pat. No. 5,681,730, describing particle-mediated transformation of gymnosperms.

In alternative embodiments, protoplasts can be immobilized and injected with a nucleic acids, e.g., an expression construct. Although plant regeneration from protoplasts is not easy with cereals, plant regeneration is possible in legumes using somatic embryogenesis from protoplast derived callus. Organized tissues can be transformed with naked DNA using gene gun technique, where DNA is coated on tungsten microprojectiles, shot 1/100th the size of cells, which carry the DNA deep into cells and organelles. Transformed tissue is then induced to regenerate, usually by somatic embryogenesis. This technique has been successful in several cereal species including maize and rice.

In alternative embodiments, a third step can involve selection and regeneration of whole plants capable of transmitting the incorporated target gene to the next generation. Such regeneration techniques rely on manipulation of certain phytohormones in a tissue culture growth medium, typically relying on a biocide and/or herbicide marker that has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee (1987) Ann. Rev. of Plant Phys. 38:467-486. To obtain whole plants from transgenic tissues such as immature embryos, they can be grown under controlled environmental conditions in a series of media containing nutrients and hormones, a process known as tissue culture. Once whole plants are generated and produce seed, evaluation of the progeny begins.

Viral transformation (transduction) may also be used for transient or stable expression of a gene, depending on the nature of the virus genome. The desired genetic material is packaged into a suitable plant virus and the modified virus is allowed to infect the plant. The progeny of the infected plants is virus free and also free of the inserted gene. Suitable methods for viral transformation are described or further detailed e. g. in WO 90/12107, WO 03/052108 or WO 2005/098004.

In alternative embodiments, after the chimeric gene is stably incorporated in transgenic plants, it can be introduced into other plants by sexual crossing or introgression. Any of a number of standard breeding techniques can be used, depending upon the species to be crossed. Since transgenic expression of the nucleic acids of the invention leads to phenotypic changes, plants comprising the recombinant nucleic acids of the invention can be sexually crossed with a second plant to obtain a final product. Thus, the seed of the invention can be derived from a cross between two transgenic plants of the invention, or a cross between a plant of the invention and another plant. The desired effects (e.g., expression of the polypeptides of the invention to produce a plant in which flowering behavior is altered) can be enhanced when both parental plants express the polypeptides, e.g., an HDC1 gene of the invention. The desired effects can be passed to future plant generations by standard propagation means.

Successful examples of the modification of plant characteristics by transformation with cloned sequences which serve to illustrate the current knowledge in this field of technology, and include for example: U.S. Pat. Nos. 5,571,706; 5,677,175; 5,510,471; 5,750,386; 5,597,945; 5,589,615; 5,750,871; 5,268,526; 5,780,708; 5,538,880; 5,773,269; 5,736,369 and 5,619,042.

In alternative embodiments, following transformation, plants are selected using a dominant selectable marker incorporated into the transformation vector. Such a marker can confer antibiotic or herbicide resistance on the transformed plants, and selection of transformants can be accomplished by exposing the plants to appropriate concentrations of the antibiotic or herbicide.

In alternative embodiments, after transformed plants are selected and grown to maturity, those plants showing a modified trait are identified. The modified trait can be any of those traits described above. In alternative embodiments, to confirm that the modified trait is due to changes in expression levels or activity of the transgenic polypeptide or nucleic acid can be determined by analyzing mRNA expression using Northern blots, RT-PCR or microarrays, or protein expression using immunoblots or Western blots or gel shift assays.

“Introgressing” means the integration of a gene in a plant's genome by natural means, i.e. by crossing a plant comprising the chimeric gene described herein with a plant not comprising said chimeric gene. The offspring can be selected for those comprising the chimeric gene.

The nucleic acids and polypeptides used to practice this invention can be expressed in or inserted in any plant cell, organ, seed or tissue, including differentiated and undifferentiated tissues or plants, including but not limited to roots, stems, shoots, cotyledons, epicotyl, hypocotyl, leaves, pollen, seeds, tumor tissue and various forms of cells in culture such as single cells, protoplast, embryos, and callus tissue. The plant tissue may be in plants or in organ, tissue or cell culture.

The invention further provides plants, plant cells, organs, seeds or tissues that have been modified so as to have an increased expression and/or activity of a protein having the activity of the protein with the amino acid sequence of SEQ ID NO. 6. when compared to a control plant. These include for example transgenic plants, plant cells, organs, seeds or tissues, comprising and expressing the nucleic acids used to practice this invention resulting in an increased expression and/or activity of an HDC1 polypeptide; for example, the invention provides plants, e.g., transgenic plants, plant cells, organs, seeds or tissues that show improved growth under (mild or moderate) stress conditions such as limiting water conditions; thus, the invention provides stress-tolerant, and particularly drought-tolerant plants, plant cells, organs, seeds or tissues (e.g., crops). The invention also provides plants, e.g., transgenic plants, plant cells, organs, seeds or tissues that show improved growth under control conditions; thus, the invention provides plants, plant cells, organs, seeds or tissues (e.g., crops) with increased biomass and/or yield and/or growth rate. The invention further provides plants, e.g., transgenic plants, plant cells, organs, seeds or tissues that show improved growth under limiting water conditions; thus, the invention provides drought-tolerant plants, plant cells, organs, seeds or tissues (e.g., crops). The invention provides plants, e.g., transgenic plants, plant cells, organs, seeds or tissues that show an accelerated flowering time; thus, the invention provides plants, plant cells, organs, seeds or tissues (e.g., crops) with an accelerated flowering time.

In an alternative embodiment, the invention further provides plants, plant cells, organs, seeds or tissues that have been modified so as to have a reduced expression and/or activity of a protein having the activity of the protein with the amino acid sequence of SEQ ID NO. 6. when compared to a control plant. These include for example transgenic plants, plant cells, organs, seeds or tissues, comprising and expressing the nucleic acids used to practice this invention resulting in a reduced expression and/or activity of an HDC1 polypeptide, for example, the invention provides plants, e.g., transgenic plants, plant cells, organs, seeds or tissues that show enhanced survival under severe stress conditions enhanced recovery after severe stress conditions. Also provided are plants, e.g., transgenic plants, that show a delayed flowering time. Preferable, the reduction in expression and/or activity of a protein having the activity of the protein with the amino acid sequence of SEQ ID NO. 6 is inducible.

The plant, plant part, plant organs and plant cell of the invention comprising a nucleic acid used to practice this invention (e.g., a transfected, infected or transformed cell) can be dicotyledonous (a dicot) or monocotyledonous (a monocot). Examples of monocots comprising a nucleic acid of this invention, e.g., as monocot transgenic plants of the invention, are grasses, such as meadow grass (blue grass, Poa), forage grass such as festuca, lolium, temperate grass, such as Agrostis, and cereals, e.g., wheat, oats, rye, barley, rice, sorghum, and maize (corn). Examples of dicots comprising a nucleic acid of this invention, e.g., as dicot transgenic plants of the invention, are cotton, tobacco, legumes, such as lupins, potato, sugar beet, pea, bean and soybean, and cruciferous plants (family Brassicaceae), such as cauliflower, rape seed, and the closely related model organism Arabidopsis thaliana. Thus, plant or plant cell comprising a nucleic acid of this invention, including the transgenic plants and seeds of the invention, include a broad range of plants, including, but not limited to, species from the genera Anacardium, Arachis, Asparagus, Atropa, Avena, Brassica, Citrus, Citrullus, Capsicum, Carthamus, Cocos, Cojfea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Manihot, Majorana, Medicago, Nicotiana, Olea, Oryza, Panieum, Pannisetum, Persea, Phaseolus, Pistachia, Pisum, Pyrus, Prunus, Raphanus, Ricinus, Secale, Senecio, Sinapis, Solarium, Sorghum, Theobromus, Trigonella, Triticum, Vicia, Vitis, Vigna, and Zea.

The invention furthermore provides propagating material created from the plant of plants cells of the invention. The creation of propagating material relates to any means know in the art to produce further plants, plant parts or seeds and includes inter alia vegetative reproduction methods (e.g. air or ground layering, division, (bud) grafting, micropropagation, stolons or runners, storage organs such as bulbs, corms, tubers and rhizomes, striking or cutting, twin-scaling), sexual reproduction (crossing with another plant) and asexual reproduction (e.g. apomixis, somatic hybridization).

In particular embodiments the plant cell described herein is a non-propagating plant cell or a plant cell that cannot be regenerated into a plant or a plant cell that cannot maintain its life by synthesizing carbohydrate and protein from the inorganics, such as water, carbon dioxide, and inorganic salt, through photosynthesis.

A transgenic plant of this invention can also include the machinery necessary for expressing or altering the activity of a polypeptide encoded by an endogenous gene, e.g a gene ecoding a functional HDC1 protein according to the invention, for example, by altering the phosphorylation state of the polypeptide to maintain it in an activated state. Transgenic plants (or plant cells, or plant explants, or plant tissues) incorporating the nucleic acids of the invention and/or expressing the polypeptides of the invention can be produced by a variety of well-established techniques as described elsewhere in this application.

A nucleic acid or polynucleotide, as used herein, can be DNA or RNA, single- or double-stranded. Nucleic acids can be synthesized chemically or produced by biological expression in vitro or even in vivo. Nucleic acids can be chemically synthesized using appropriately protected ribonucleoside phosphoramidites and a conventional DNA/RNA synthesizer. Suppliers of RNA synthesis reagents are Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). In connection with the chimeric gene of the present disclosure, DNA includes cDNA and genomic DNA.

The terms “protein” or “polypeptide” as used herein describe a group of molecules consisting of more than 30 amino acids, whereas the term “peptide” describes molecules consisting of up to 30 amino acids. Proteins and peptides may further form dimers, trimers and higher oligomers, i.e. consisting of more than one (poly)peptide molecule. Protein or peptide molecules forming such dimers, trimers etc. may be identical or non-identical. The corresponding higher order structures are, consequently, termed homo- or heterodimers, homo- or heterotrimers etc. The terms “protein” and “peptide” also refer to naturally modified proteins or peptides wherein the modification is effected e.g. by glycosylation, acetylation, phosphorylation and the like. Such modifications are well known in the art.

As used herein “comprising” is to be interpreted as specifying the presence of the stated features, integers, steps or components as referred to, but does not preclude the presence or addition of one or more features, integers, steps or components, or groups thereof. Thus, e.g., a nucleic acid or protein comprising a sequence of nucleotides or amino acids, may comprise more nucleotides or amino acids than the actually cited ones, i.e., be embedded in a larger nucleic acid or protein. A chimeric gene comprising a nucleic acid which is functionally or structurally defined, may comprise additional DNA regions etc.

Unless stated otherwise in the Examples, all recombinant DNA techniques are carried out according to standard protocols as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy, jointly published by BIOS Scientific Publications Ltd (UK) and Blackwell Scientific Publications, UK. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

All patents, patent applications, and publications or public disclosures (including publications on internet) referred to or cited herein are incorporated by reference in their entirety.

The sequence listing contained in the file named “BCS13-2001_ST25”, which is 376 kilobytes (size as measured in Microsoft Windows®), contains 41 sequences SEQ ID NO: 1 through SEQ ID NO: 55, is filed herewith by electronic submission and is incorporated by reference herein.

The invention will be further described with reference to the examples described herein; however, it is to be understood that the invention is not limited to such examples.

### SEQUENCE LISTING

SEQ ID NO. 1: Promoter region of the Arabidopsis thaliana HDC1 gene
SEQ ID NO. 2: overexpression vector pMDC32 35S HDC
SEQ ID NO. 3: overexpression vector pUB-DEST Ubi10 HDC1
SEQ ID NO. 4: Amino acid sequence Saccharomyces cerevisiae Rxt3 aa
SEQ ID NO. 5: Nucleotide sequence of HDC1 from Arabidopsis thaliana
SEQ ID NO. 6: Amino acid sequence of HDC1 from Arabidopsis thaliana
SEQ ID NO. 7: Nucleotide sequence of HDC1 from Arabidopsis lyrata
SEQ ID NO. 8: Amino acid sequence of HDC1 from Arabidopsis lyrata
SEQ ID NO. 9: Nucleotide sequence of HDC1 from Populus trichocarpa
SEQ ID NO. 10: Amino acid sequence of HDC1 from Populus trichocarpa
SEQ ID NO. 11: Nucleotide sequence of HDC1 from Medicago truncatula
SEQ ID NO. 12: Amino acid sequence of HDC1 from Medicago truncatula
SEQ ID NO. 13: Nucleotide sequence of HDC1 from Vitis vinifera
SEQ ID NO. 14: Amino acid sequence of HDC1 from Vitis vinifera
SEQ ID NO. 15: Nucleotide sequence of HDC1 from Ricinus communis
SEQ ID NO. 16: Amino acid sequence of HDC1 from Ricinus communis
SEQ ID NO. 17: Nucleotide sequence of HDC1 from Oryza sativa
SEQ ID NO. 18: Amino acid sequence of HDC1 from Oryza sativa
SEQ ID NO. 19: Nucleotide sequence of HDC1 from Oryza sativa
SEQ ID NO. 20: Amino acid sequence of HDC1 from Oryza sativa
SEQ ID NO. 21: Nucleotide sequence of HDC1 from Brachypodium distachyon
SEQ ID NO. 22: Amino acid sequence of HDC1 from Brachypodium distachyon
SEQ ID NO. 23: Nucleotide sequence of HDC1 from Sorghum bicolor
SEQ ID NO. 24: Amino acid sequence of HDC1 from Sorghum bicolor
SEQ ID NO. 25: Nucleotide sequence of HDC1 from Sorghum bicolor
SEQ ID NO. 26: Amino acid sequence of HDC1 from Sorghum bicolor
SEQ ID NO. 27: Nucleotide sequence of HDC1 from Zea mays
SEQ ID NO. 28: Amino acid sequence of HDC1 from Zea mays
SEQ ID NO. 29: Nucleotide sequence of HDC1 from Glycine max
SEQ ID NO. 30: Amino acid sequence of HDC1 from Glycine max
SEQ ID NO. 31: Nucleotide sequence of HDC1 from Glycine max
SEQ ID NO. 32: Amino acid sequence of HDC1 from Glycine max
SEQ ID NO. 33: Nucleotide sequence of HDC1 from Glycine max
SEQ ID NO. 34: Amino acid sequence of HDC1 from Glycine max
SEQ ID NO. 35: Nucleotide sequence of HDC1 from Glycine max
SEQ ID NO. 36: Amino acid sequence of HDC1 from Glycine max
SEQ ID NO. 37: Nucleotide sequence of HDC1 from Triticum aestivum
SEQ ID NO. 38: Amino acid sequence of HDC1 from Triticum aestivum
SEQ ID NO. 39: Nucleotide sequence of HDC1 from Solanum lycopersicum
SEQ ID NO. 40: Amino acid sequence of HDC1 from Solanum lycopersicum
SEQ ID NO. 41: Amino acid sequence of HDC1 from Oryza sativa
SEQ ID NO. 42: hdc1-1 flanking sequence forward primer (genotyping)
SEQ ID NO. 43: hdc1-1 flanking sequence reverse primer (genotyping)
SEQ ID NO. 44: hdc1-1 left border forward primer (genotyping)
SEQ ID NO. 45: hdc1-1 left border reverse primer (genotyping)
SEQ ID NO. 46: HDC1 paid forward primer (RT-PCR/qPCR)
SEQ ID NO. 47: HDC1 paid reverse primer (RT-PCR/qPCR)
SEQ ID NO. 48: HDC1 pair2 forward primer (RT-PCR/qPCR)
SEQ ID NO. 49: HDC1 pair2 reverse primer (RT-PCR/qPCR)
SEQ ID NO. 50: HDC1 pair3 forward primer (RT-PCR/qPCR)
SEQ ID NO. 51: HDC1 pair3 reverse primer (RT-PCR/qPCR)
SEQ ID NO. 52: HDC1 pair4 forward primer (RT-PCR/qPCR)
SEQ ID NO. 53: HDC1 pair4 reverse primer (RT-PCR/qPCR)
SEQ ID NO. 54: Nucleotide sequence of HDC1 from Arabidopsis thaliana codon-optimized for overexpression in wheat
SEQ ID NO. 55: overexpression vector pTVE704

### EXAMPLES

### Example 1

### Experimental Procedures

### Plant Materials

All transgenic lines for HDC1 were generated in our laboratory in Arabidopsis thaliana Col-0 background. The stable homozygous knockout line hdc1-1 was obtained from progeny of GABI-Kat line 054G03. Stable, homozygous complementation lines were identified from the progeny of hdc1-1 plants transformed with genomic HDC1 including the native promoter (see cloning procedures). Stable, homozygous HDC1-overexpressing lines were generated from the progeny of wildtype Col-0 plants transformed with HDC1 under the control of 35-S or Ubiquitin-10 promoters (see cloning procedures). Seeds for 35S::HDA6 (Gu et al., 2011, PLoS Genet. 7) and axe1-5 (Probst et al., 2004, Plant Cell 16, 1021-1034) were kindly provided by Yuehui He and Ortrun Mittelsten Scheid.

Growth Conditions and Treatments

All experiments were carried out in controlled growth rooms at a temperature of 20-22° C. and a light intensity of 120-150 μmol PAR. Plants were grown either in long days (16 h light) or in short days (10 h light) as indicated in text and figure legends. Seeds of A. thaliana wildtype and transgenic lines were sterilized, stratified and germinated on soil or on agar plates. Agar plates contained half strength Murashige & Skoog (MS) media with 1% sucrose and 0.8% agar at pH 5.7. For germination assays media were supplemented with NaCl, ABA (cat. A1049,SIGMA), PAC (Fluka cat. 46046) or TSA (SIGMA cat.T8852) at the concentrations given in the figures. Germination rate was scored on day 6 after sowing by counting seedlings that had developed green cotyledons. Experiments with adult plants were carried out on soil or in hydroponic culture. For the latter, seeds were germinated on agar plates and 2-3 weeks old seedlings were placed perforated lids of black 1-litre plastic containers. The growth medium consisted in a minimal sufficient nutrient medium (Kellermeier et al., 2013, PLoS Genet. 7). For salt treatment NaCl powder was stirred directly into the growth container to obtain the desired concentration (as stated in the figures). Control media were stirred without adding NaCl. For controlled drought experiments, plants were grown on soil in pots according to a randomized design. Using previously reported methodology (Granier et al., 2006, New Phytologist 169:623-635; Skirycz et al., 2011, Nat. Biotech. 29:212-214), controlled watering was used to impose moderate water stress. After 14 days of plant growth in well-watered soil, watering was reduced so that the relative soil water content of the stressed plants was maintained at 50% of the normal watering regime. Control plants were watered normally.

### Cloning Procedures

Entry clones with full length HDC1, HDA6, HDA19 and AtSIN3 with or without stop codon were generated by PCR amplification using primers that contained attB1 and attB2 sites or attB3 and attB4 as 5′ modifications. Gel-purified PCR products were introduced into pDONR207/221 (Life Technologies) using BP-clonase II according to the manufacturer's instructions and transferred to destination vectors by recombination using LR-clonase II (Life Technologies). The reaction product was used to transform Top10 bacterial cells. Antibiotic marker-resistant colonies were isolated and verified by restriction digest analysis and sequencing. The following plasmids were generated and used in this study: 35S::HDA6/HDA19-RFP in pB7RWG2, HDC1 (646 bp upstream) promoter in pMDC163, HDC1 gDNA (including 646 bp upstream sequence) in pMDC123, 2×35S::HDC1 in pMDC032 (Curtis and Grossniklaus, 2003, Plant Physiol. 133:462-9), Ubi10::HDC1 in pUB-Dest, 35S::GFP-HDC1 in pH7WGF2 (Karimi et al., 2002, Trends Plant Sci 7:193-195), Ubi10::GFP-HDC1 pUBN-GFPDest (Grefen et al., 2010, Plant J 64:355-365), 35S::nYFP-HDC1/cYFP-HDA6/HDA19/SIN3 in pBiFCt-2in1-NN, 35S::nYFP-SIN3/cYFP-HDA19 in pBiFCt-2in1-NN (Grefen and Blatt, 2012, Biotechniques 53:311-314).

### Antibodies

HDC1 antibody was raised in rabbit (Agrisera) using a synthetic peptide matching amino acids 341-356 in the HDC1 sequence, and affinity purified. An extra cysteine was added to the N-terminus to improve binding capacity. H3K9/K14Ac and H3 antibodies were purchased from Diagenode (pAb-005-044) and Abcam (ab1791). His-tag antibody was obtained from NEB (#2366).

### Plant Transformation

Plasmids were inserted by heat shock into Agrobacterium tumefaciens strain GV3101 pMP90 (Koncz and Schell, 1986, Mol. Gen. Genet. 204: 383-396). Agrobacterium-mediated transformation of A. thaliana was performed by the floral-dip method (Clough and Bent, 1998, Plant J. 16, 735-743). Homozygous T₂ progenies were used for germination tests. Agrobacterium-mediated transient transformation of N. tabacum and N. benthamiana was achieved by leaf infiltration (Geelen et al., 2002, Plant Cell 14: 387-406). For ratiometric BiFC assays and co-localisation studies, each construct was co-expressed with p19 protein of tomato blushy stunt virus, encoding for a suppressor of gene silencing (Voinnet et al., 2003, Plant Journal 33, 949-956).

### Polymerase Chain Reaction

Total genomic DNA was extracted according to (Edwards et al., 1991, Nucleic Acids Research 19, 1349-1349). All the PCR reactions were performed with 0.4 units of Taq polymerase (Promega cat. M8301). Total RNA was extracted using hot phenol (Schmitt et al., 1990, Nucleic Acids Research 18, 3091-3092). cDNA was obtained with Quantitect Reverse Transcription kit (Qiagen) following manufactures procedure. Quantitative PCR was performed on MX3000 sequence detection system (Agilent) with Brilliant III Ultra Fast SYBR QPCR Master Mix n (Agilent). Primer sequences are provided in the sequence listing as SEQ IDs 43-53.

### ChIP

Chromatin extraction and immunoprecipitation (ChIP) were carried out following published protocols ((Gendrel et al., 2002, Science 297, 1871-1873; Saleh et al., 2008, Plant Cell 20, 568-579). In brief, tissue samples were incubated in 1% (w/v) formaldehyde for 15 min under vacuum. Cross-linking was stopped by adding 125 mM glycine, and tissues were rinsed, blotted dry and frozen. Diluted chromatin extracts were incubated with antibody against H3K9/K14Ac (Diagenode pAb-005-044) following the manufacture instructions. Immunoprecipitated chromatin-DNA (IP-DNA) or input chromatin-DNA was reverse cross-linked and residual protein was removed by proteinase K treatment. DNA was recovered by phenol/chloroform extraction and ethanol precipitation. DNA then was re-suspended and purified by MinElute Reaction Cleanup kit (QIAGEN). Before proceeding to ChIP-qPCR, DNA samples were amplified using GenomePlex Complete Whole Genome Amplification (WGA2, Sigma-Aldrich) following the manufacturer's protocol.

Protein Extraction and Western Blotting

Nuclei-enriched protein extracts were prepared according to published a published protocol (Gendrel et al., 2002, supra). The chromatin was extracted twice with 0.4M H2SO4 and protein precipitated with 20% trichloroacetic acid. All buffers were supplemented with 100 mM PMSF and proteinase inhibitors (Complete Mini, Roche UK). Samples were boiled and loaded onto SDS-PAGE gels. After transfer to PVDF membrane (IPVH00010, Millipore), Ponceau S staining (P3504, Sigma-Aldrich) was carried out. HDC1 antibody was incubated overnight in a dilution of 1:4000. Secondary rabbit antibody conjugated with horseradish peroxidase (Roche) was incubated with the membrane for at least 1 h. Proteins were detected using the ECL+system (RPN2132, Amersham).

### Production of Recombinant Tagged Protein and GST Pull Down Assays

GST- or His-tagged proteins were expressed in E. coli BL21 cells. Following induction with 1 mM IPTG cells were harvested and sonicated in lysis buffer. The soluble HDC1-His, GST-HDA6 and GST-HDA19 proteins were affinity-purified using the Ni-NTA (Sigma) and Glutathione-Sepharose resin (GE Healthcare) according to the manufacturer′ instructions. For pull-down assays, GST-tagged proteins were bound to Glutatione-Sepharose resin and applied to a microcolumn. Recombinant HDC1-His or nuclei-enriched plant lysates (Gendrel et al., 2002, supra) were combined with 1× protein inhibitor (Complete Mini, 11836153001, Roche, UK) in Tris-NaCl buffer. Samples were incubated overnight on ice. After several washes, pulled down protein was eluted in 1× Laemmli Buffer.

### GUS Assay

Plants tissues from independent primary transformants expressing HDC1 promoter::GUS were infiltrated in a solution containing 0.1M NaPO4, 10 mM EDTA, 0.1% Triton, 1 mM K3Fe(CN)6 and 2 mM X-GLUC. The samples were incubated overnight at 37° C., followed by 70% ethanol washes at 65° C. every two hours to remove the excess to blue coloration. Photos were taken on a stereo microscope.

### Confocal Microscopy

Fluorescence in tobacco epidermal cells was assessed two days post infiltration using a CLSM-510-META-UV confocal microscope (Zeiss, Jena). For single protein localization GFP fluorescence was excited at 488 nm with light from an Argon laser and collected after passage through an NFT545 dichroic mirror with a 505 nm long pass filter. For co-localization experiments GFP fluorescence was collected with a 505-530 band pass filter. RFP fluorescence was excited at 543 nm with light from a Helium Neon laser and was collected after passage through an NFT545 dichroic mirror and a 560-615 nm band pass filter. YFP fluorescence was excited at 514 nm with light from Argon laser and was collected using lambda mode between 520-550 nm. Co-localization plane and line scans were evaluated using Zeiss LSM 510 AIM software (v3.2).

### Determination of Abscisic Acid (ABA)

ABA in methanol-extracts from dried leaf sample was quantified by LC-MS (Page et al., 2012) at the University of Exeter Mass Spectrometry Facility (Exeter, UK) using 1200 series HPLC (Agilent Technologies, 3.5 μm, 2.1×150 mm Eclipse Plus C18 column) and a 6410B enhanced sensitivity triple quadruple mass spectrometer (Agilent Technologies). [²H6] (+)-cis, trans-abscisic acid, (Chemlm Ltd, Czech Republic) was included as a standard.

### Accession Numbers of Genes

ABA1 (ABA DEFICIENT 1): AT5G67030; ABA3(ABA DEFICIENT 3): AT1G16540; ABI3 (ABA INSENSITIVE 3): AT3G24650; AFP3 (ABI FIVE BINDING PROTEIN) 3: AT3G29575; DR4 (DROUGHT-REPRESSED 4): AT1G73330; FLC (FLOWERING LOCUS C): AT5G10140; FUS3 (FUSCA3): AT3G26790; HDC1 (HISTONE DEACETYLATION COMPLEX 1): AT5G08450; HDA6 (HISTONE DEACETYLASE 6): AT5G63110; HDA19 (HISTONE DEACETYLASE 19): AT4G38130; LEC1 (LEAFY COTYLEDON 1): AT1G21970; PYL4 (PYR1-LIKE 4): AT2G38310; RAB18 (RESPONSIVE TO ABA 18): AT5G66400; RD29A (RESPONSIVE TO DESSICATION 29): AAT1G16540; RD29B (RESPONSIVE TO DESSICATION 29B): AT5G52300; SIN3 (SIN3-LIKE 3): AT1G24190.

### Example 2

### HDC1 is a Non-Redundant, Ubiquitous, Nuclear Protein

HDC1 (At5g08450) is a single-copy gene in A. thaliana. Predicted splice variants only differ in the upstream UTR. Unique HDC1 homologues are also present in all other plant species for which genome information is currently available, including important crops such as maize and rice (FIG. 1A). The ˜900 amino-acid long sequence of the predicted plant HDC1 proteins contains a ˜300 amino-acid long sequence in the C-terminal half that is highly similar to Rxt3 proteins, which are ubiquitously present in lower eukaryotes but remain functionally uncharacterized (alignment in FIG. 1C). Particularly high sequence similarity occurs in a Pfam signature (PF08642) labeled as ‘histone de-acetylation Rxt3’ (box in FIG. 1C). The term derives from biochemical evidence that yeast Rxt3 co-elutes with the LRpd3 complex (Carrozza et al., 2005, Cell 123, 581-592.) but the region has no homology to catalytic domains of histone deacetylases. Based on sequence similarity no obvious function can be assigned to this or any other part of the HDC1 sequence. The more variable extended N-terminal part of HDC1 has no counterpart in non-plant genomes. Sequence extension from Rxt3 to HDC1 occurred between algae and higher plants with mosses showing intermediate length (see sequence alignment in FIG. 1C).

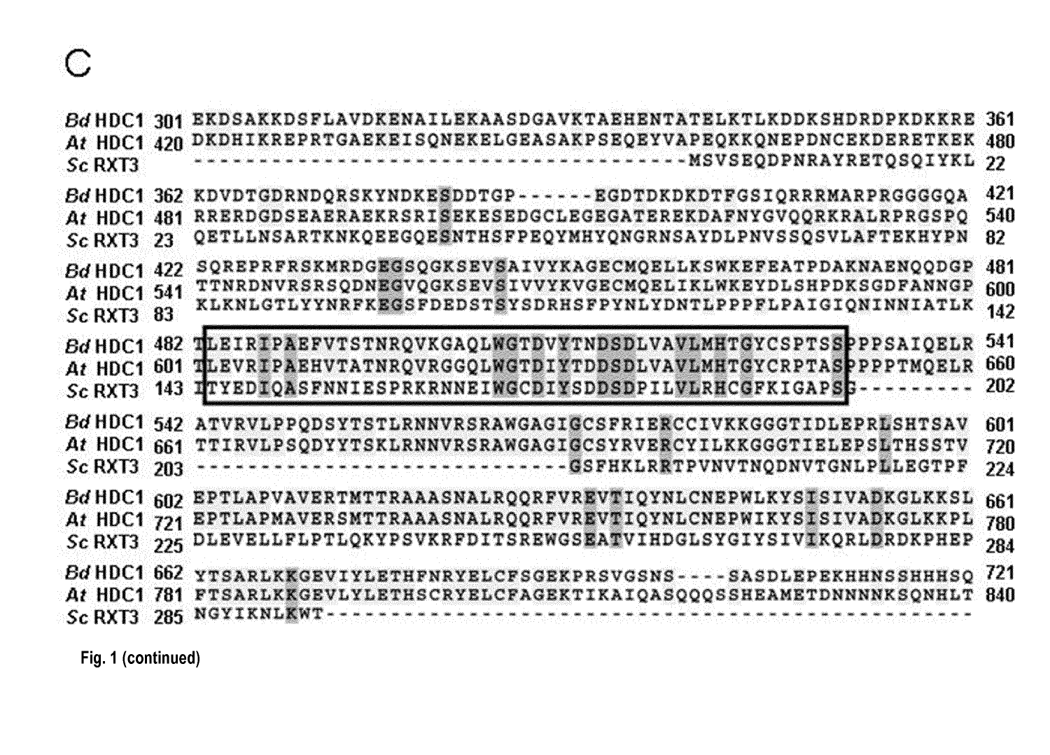

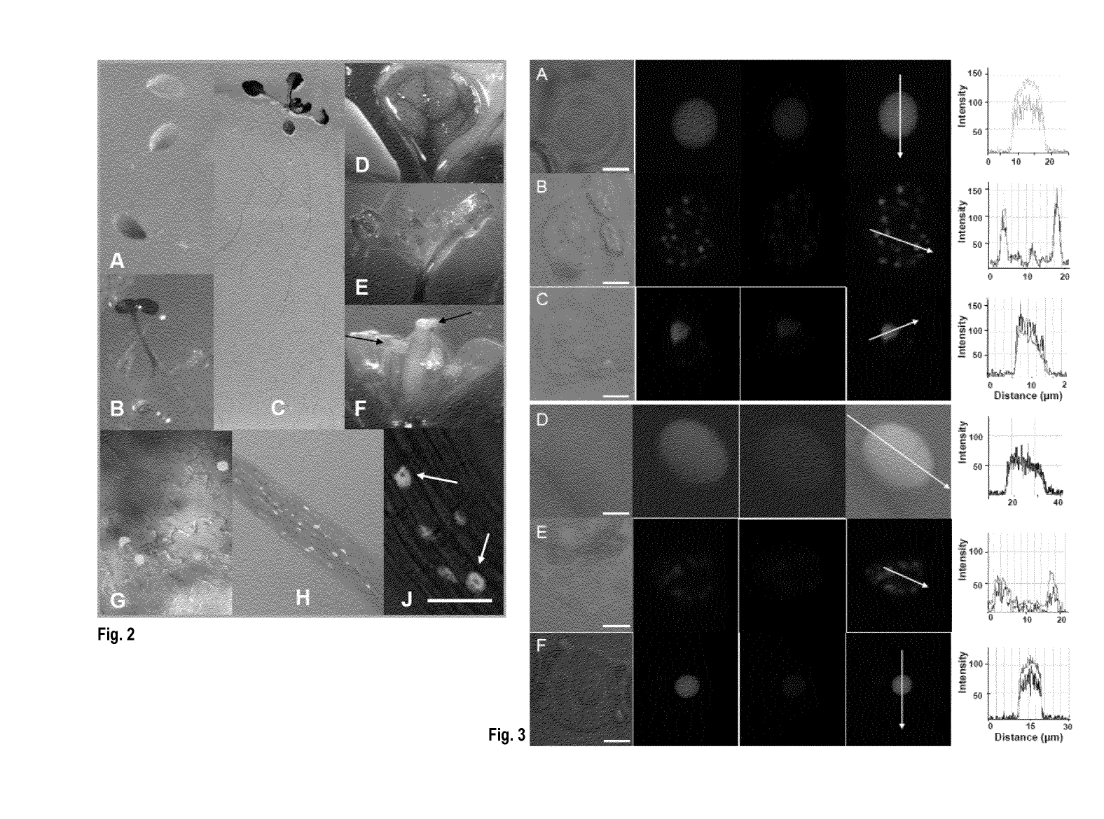

The notion of a conserved non-redundant function of HDC1 is supported by ubiquitous expression within the plant. Histochemical analysis of stable A. thaliana lines expressing β-glucuronidase (GUS) under the control of the HDC1 promoter revealed HDC1-promoter activity in all vegetative tissues, including seed, root, cotyledon, rosette leaf and flower bud (FIG. 2, A-E). However, GUS was not detected inside anthers and stigmas (FIG. 2, F), indicating that HDC1 is silenced during reproduction. This is in accordance with a general re-setting of chromatin status during reproduction (Paszkowski and Grossniklaus, 2011, Current Opinion in Plant Biology 14, 195-203).

Microscopical analysis of a green fluorescent protein (GFP)-HDC1 fusion protein in transiently expressing tobacco plants and in stable transgenic A. thaliana plants showed exclusive presence of HDC1 in the nucleus (FIG. 2, G, H) but not in the nucleolus (FIG. 2, J).

### Example 3

### HDC1 Physically Interacts with HDA6 and HDA19 and Promotes Histone Deacetylation

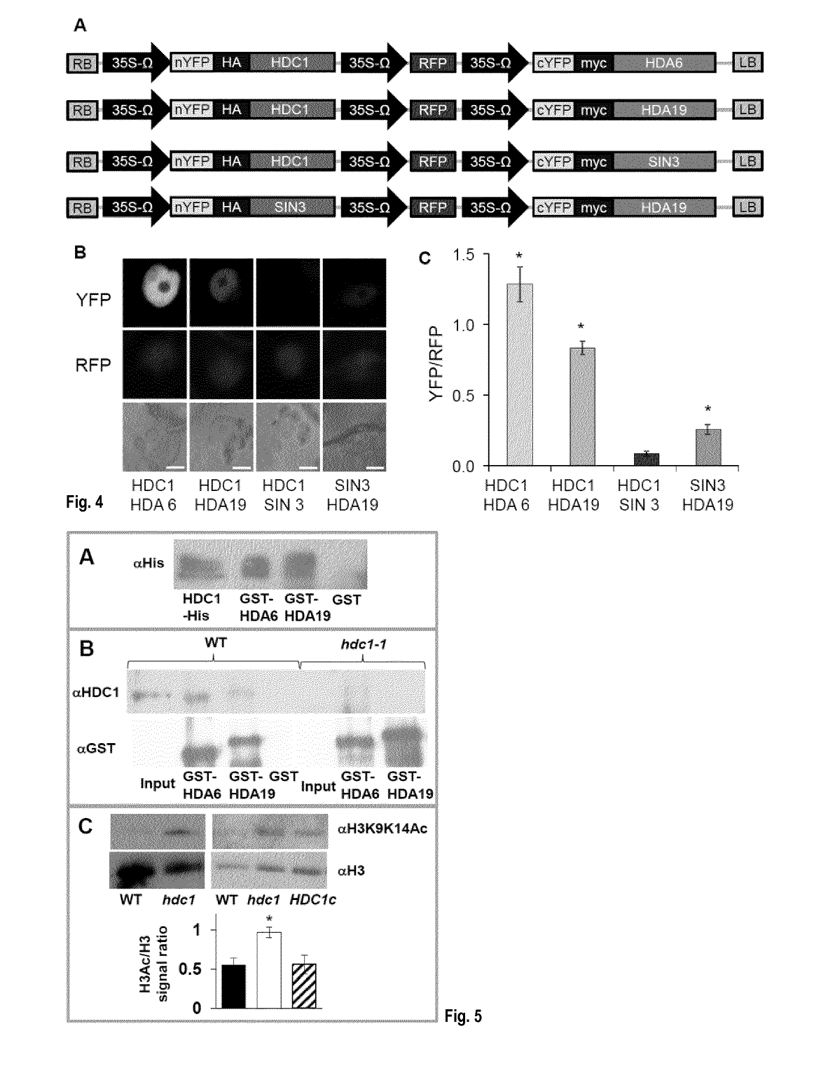

To investigate whether HDC1 is a member of HDAC protein complexes in plants we tested co-localization and direct interaction of HDC1 with known HDACs of A. thaliana. Co-expression of full-length GFP-HDC1 with red fluorescent protein (RFP)-HDA6 or RFP-HDA19 in epidermal tobacco cells indicated tight co-localization of HDC1 with HDA6 and HDA19 in different locations within the nucleus (FIG. 3). Direct interaction was investigated by bimolecular fluorescence complementation (BiFC). To avoid misinterpretation of background fluorescence we used a new ratiometric BiFC assay (Grefen and Blatt, 2012, supra) in which N- and C-terminal halves of yellow fluorescent protein (YFP), fused to HDC1 and HDA6/19 respectively, and a full-length RFP, are expressed from a single vector FIG. 4A). In RFP-producing cells, a strong YFC signal was recorded for HDA6 and for HDA19, indicating successful BiFC and hence interaction of HDC1 with both HDACs. BiFC was also successful when HDA19 was co-expressed with Sin3-like protein 3 (SNL3, AtSin3) previously shown to interact with HDA19 in yeast-2-hydrid assays (Song et al., 2005, supra). By contrast, no YFP signal was recorded for HDC1 and AtSin3 indicating that HDC1 does not interact with all HDAC complex proteins. Normalization of the obtained YFP signal to the RFP signal from the same cell (FIG. 4B) provided statistically significant, quantitative evidence for a strong and specific interaction of HDC1 with the two deacetylases in the heterologous system (FIG. 4C).

In vitro pull-down experiments using GST- and His-tagged recombinant proteins further confirmed the ability of HDC1 to physically interact with HDA6 and HDA19 (FIG. 5A). Using GST-HDA6 as bait, HDC1 was pulled down in nuclei-enriched protein samples obtained from leaves of mature A. thaliana plants (FIG. 5B). [Note that a triple band of HDC1 seen in the in-vitro pull down samples was not seen here indicating stable post-translational modifications in the heterologous system but not in planta.] Considerably less HDC1 was pulled down when GST-HDA19 was used as bait. HDC1 was not recovered in pull-down assays with GST alone. No HDC1 was detected when the same assays were performed with protein extract from a T-DNA insertion knockout line, hdc1-1 (for mutant description see below).

To test whether HDC1 had an influence on histone deacetylation activity in the plant, we probed leaf protein extracts from wildtype and mutant lines with a commercial antibody that recognizes acetylated lysines 9 and 14 in histone 3 (anti-H3K9K14ac), a predominant target of HDA6 (To et al., 2011, supra). As shown in FIG. 5C, hdc1-1 knockout plants produced a significantly higher H3K9K14ac:H3 signal ratio than wildtype plants, indicating higher levels of the acetylated form of H3 over the de-acetylated form. Expression of the genomic sequence of HDC1 under its own promoter in the hdc1-1 background (HDC1c) reverted this phenotype; H3K9K14ac:H3 in the complementation line was similar to wildtype (FIG. 5C). We conclude that HDC1 interacts with histone deacetylases and is required for histone deacetylase activity in planta.

### Example 4

### Mutant Lines for Functional Characterization of HDC1

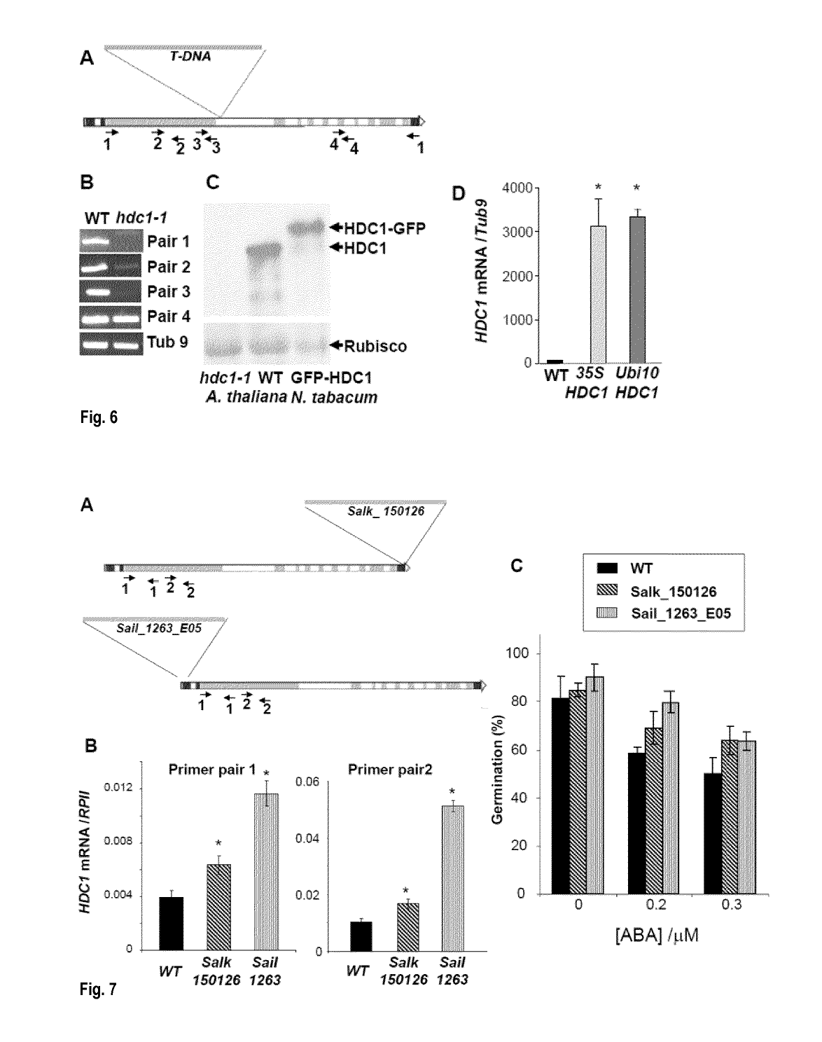

To investigate physiological functions of HDC1 we generated several homozygous lines from currently available A. thaliana lines with T-DNA-insertions in HDC1 coding sequence or UTRs (SALK043645, SALK 150126C, SAIL1263E05 and GABI-Kat 054G03, all in Col-0 background). Only one of these, hdc1-1 derived from GABI-Kat 054G03, with a TDNA-insertion in the first intron, proved to be a true knockout of HDC1 at transcript and protein level (FIG. 6A-C). HDC1 transcript levels in the other T-DNA insertion lines were similar to those in wildtype or even higher FIG. 7A,B). Some partial mRNA but no HDC1 protein (full-length or partial) was detected in hdc1-1 plants (Supplemental fig. S2C). HDC1c complementation lines were obtained by expressing genomic HDC1 under its own promoter (646 bp upstream sequence) in hdc1-1 background. We also produced stable homozygous HDC1-overexpressing lines in Col-0 background using either 35-S or Ubiquitin-10 promoter (HDC1-OX1 and HDC1-OX2 respectively). Both lines produced approximately 30-fold higher HDC1 mRNA levels than Col-0 wildtype FIG. 6D).

### Example 5

### HDC1 Determines the Set Point of ABA Sensitivity During Germination

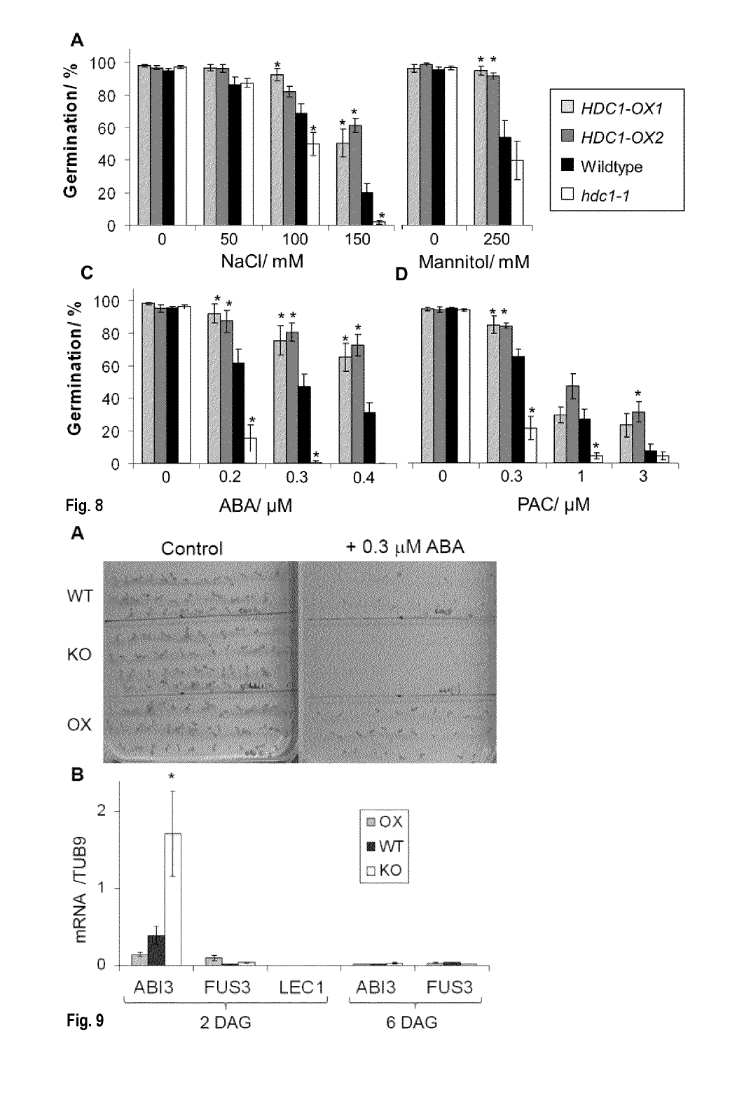

It was previously reported that hda6 and hda19 mutant lines are hypersensitive to ABA during germination (Chen et al., 2010, supra; Chen and Wu, 2010, Plant Signal Behay. 5, 1318-1320). Germinating seeds arrest growth and development if they encounter low water potentials in the environment (Finkelstein et al., 2008, In Annual Review of Plant Biology (Palo Alto: Annual Reviews), pp. 387-415). The post-imbibition response is mediated by ABA and can be mimicked by external application of ABA. Gibberellin (GA) antagonizes ABA in this response and hence seedling growth arrest also occurs if the GA-biosynthesis inhibitor paclobutrazol (PAC) is applied (Daszkowska-Golec, 2011, supra). To test a function of HDC1 in this process seeds of A. thaliana wildtype, hdc1-1, and HDC1-OX lines were imbibed to break dormancy, and subsequently plated out on agar plates containing different concentrations of NaCl, mannitol, ABA or PAC. A cumulative germination rate (encompassing all post-imbibition stages of seedling development) was scored as the number of seedlings that had developed cotyledons after 6 days. In control conditions, all lines germinated similarly well (close to 100%) and germinated seedlings were similar in size and shape (FIG. 8, FIG. 9). All lines showed a decrease in germination rates with increasing concentrations of NaCl, mannitol, ABA or PAC, however, compared to wildtype, hdc1-1 was significantly more sensitive whereas the OX lines were significantly less sensitive to the treatments. Hyposensitivity was observed in both OX lines, independent of promoter or insertion site. Homozygous lines derived from SALK 150126C, SAIL1263E05 displayed similar or slightly decreased ABA-sensitivity during germination in accordance with a moderate increase of HDC1 mRNA in these lines (FIG. 7C). We conclude that the expression level of HDC1 quantitatively determines the set point of ABA-sensitivity in germinating seeds.

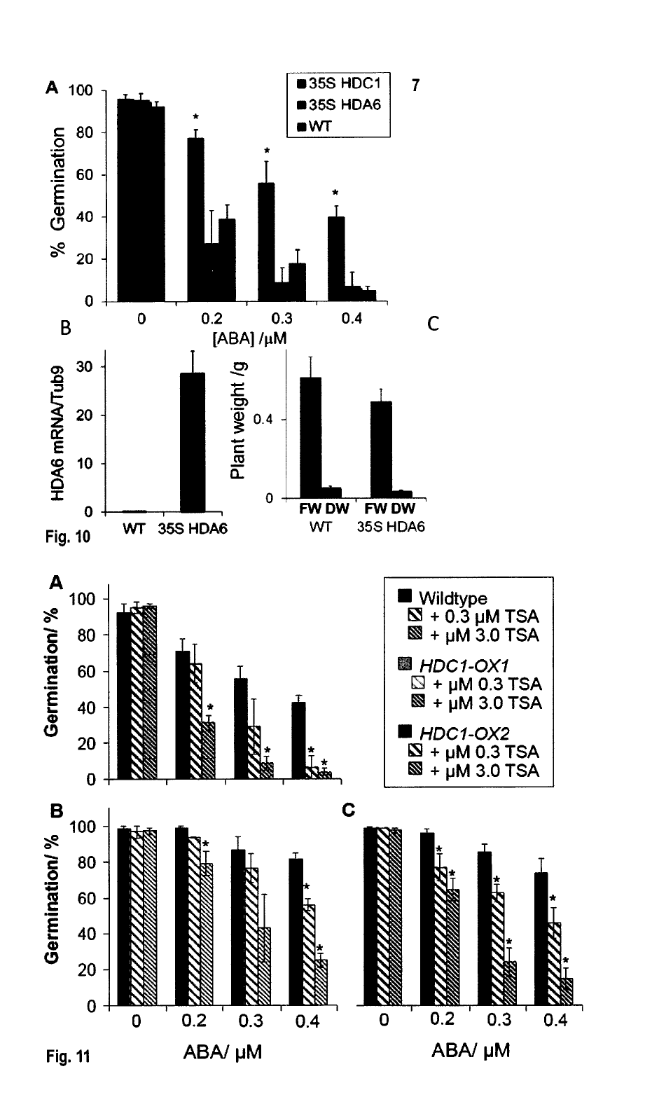

The fact that HDC1 over-expression had a de-sensitizing effect on ABA-dependent germination was interesting because no physiological phenotypes have been reported for HDA6 overexpression to date. We therefore assessed ABA-sensitivity in seedlings of an HDA6-overexpressing line previously generated for biochemical studies (Gu et al., 2011, supra). 35S::HDA6 seedlings showed similar ABA-sensitivity as wildtype plants, and they were considerably more sensitive to ABA than HDC1-OX seedlings despite a similar increase in transcript level (FIG. 10A, B).

To test whether histone deactylation was required for ABA-dependence of seed germination and for the effect of HDC1 on this process, we subjected germinating seeds to the histone deacetylase inhibitor trichostatin A (TSA). Unlike higher TSA concentrations tested before (Tanaka et al., 2008, Plant Physiol. 146:149-161), the low-micromolar concentrations of TSA applied in our experiments had no effect on seed germination in the absence of ABA (FIG. 11). Nevertheless, TSA increased the ABA-sensitivity of wildtype plants in a dose-dependent manner, with 0.3 μM producing a significant effect at 0.2 μM ABA and 3 μM TSA producing a significant effect at 0.4 μM ABA. Furthermore, addition of TSA increased ABA-sensitivity of the HDC1-overexpressing lines. Thus ABA-sensitivity of germinating seeds and de-sensitization of seedlings towards ABA by HDC1-overexpression depend on the catalytic activity of histone deacetylases.

### Example 6

### HDC1 does not Impact on Vegetative Development but is Required for Flowering

Several developmental phenotypes have been reported for HDAC mutants. For example, hda6/hda19 double mutants display embryonic structures on mature leaves and do not repress embryo-specific transcription factors such as LEC1, FUS3 and ABI3 after germination (Tanaka et al., 2008, supra). By contrast, leaves of hdc1-1 plants were normal and LEC1 and FUS3 were effectively repressed already two days after germination (DAG, FIG. 9). ABI3 transcript was still present at 2 DAG, with hdc1-1 plants expressing higher levels and HDC1-OX plants expressing lower levels than wildtype plants, but was reduced to very low levels in all lines by 6 DAG. We conclude that in control conditions HDC1 is not required for successful progression of seedlings into the vegetative growth phase.

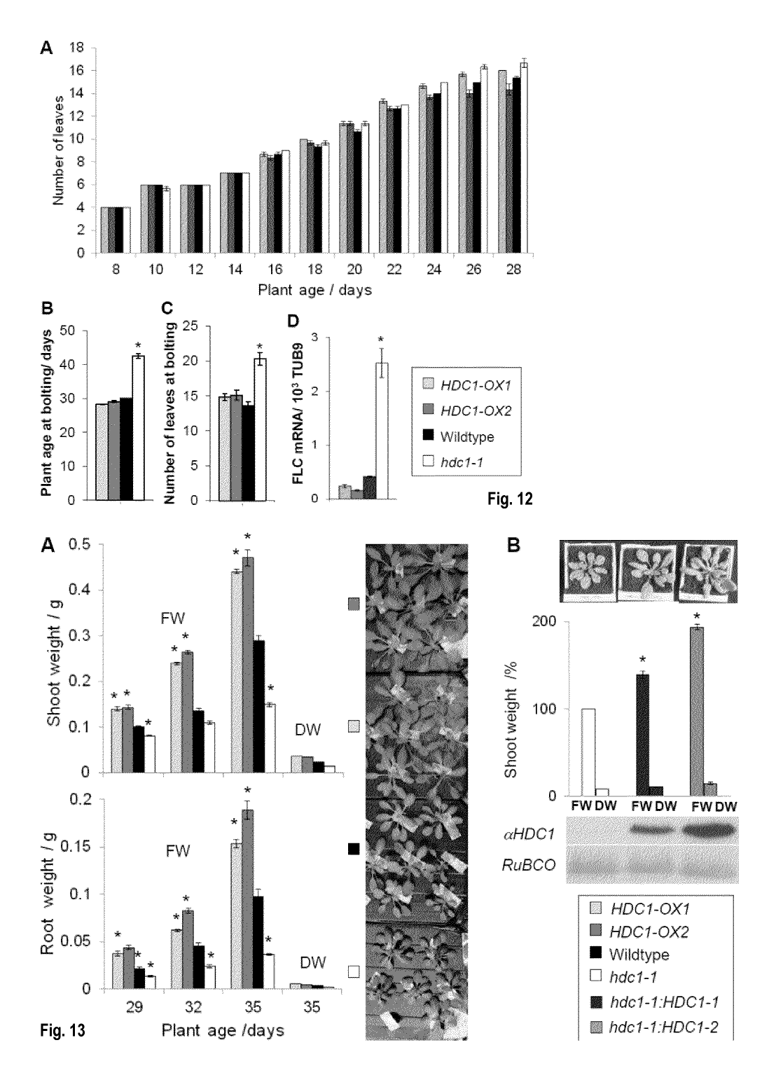

During vegetative growth, leaf development was normal in hdc1-1 and HDC1-OX plants. New leaves appeared at a similar rate in all lines (FIG. 12A). When grown in long day conditions, wildtype and HDC1-OX plants started to bolt within 4 weeks whereas hdc1-1 plants continued to produce rosette leaves and flowered approximately 2 weeks later (FIG. 12B) at considerably higher rosette leaf number (FIG. 12C). The flowering phenotype was reflected in a high transcript level of the flowering inhibitor FLC in hdc1-1 plants knockout plants on day 28 compared to low levels in the wildtype and HDC1-OX plants (FIG. 12D). It can be concluded that HDC1 does not impact on vegetative development but is required for the transition to the reproductive stage.

### Example 7

### HDC1 Promotes Plant Growth

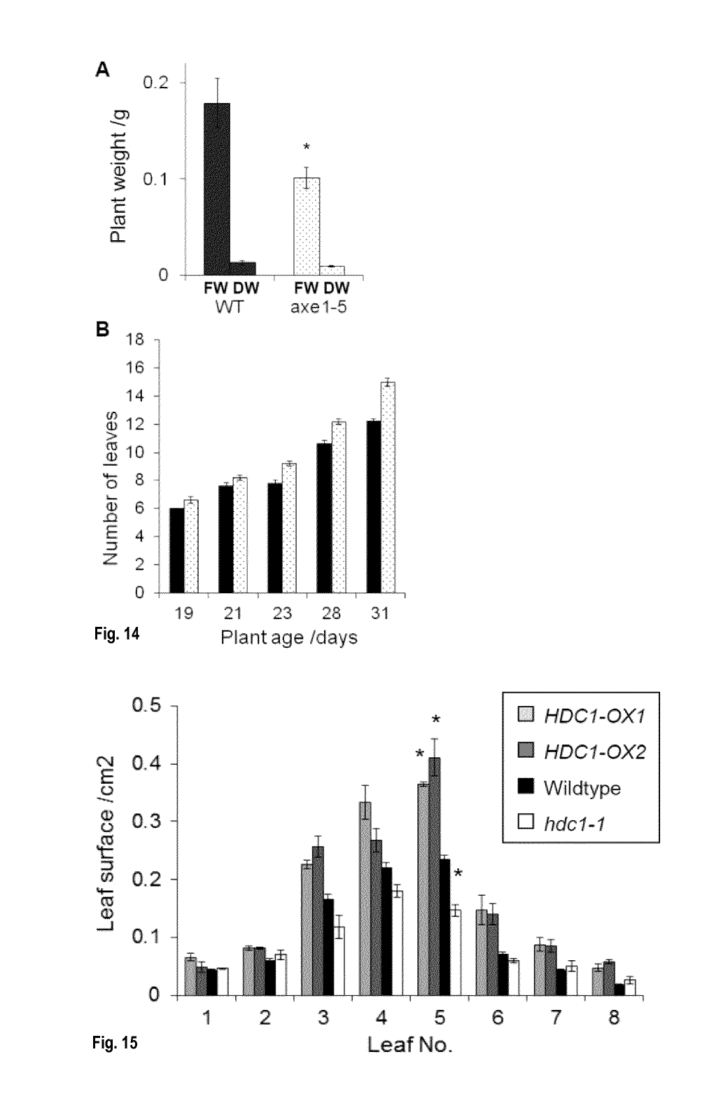

Despite normal vegetative development, HDC1 mutants showed a clear growth phenotype (FIG. 13). Differences in leaf expansion became apparent within 2 weeks after germination (FIG. 14). Significant differences of shoot and root weights between the lines were recorded in older plants, particularly when the vegetative growth phase was extended by applying short-day conditions (FIG. 13). With a similar number of leaves, 4-weeks old HDC1-OX plants had produced 20% more and hdc1-1 plants had produced 10% less fresh weight than wildtype plants, and the differences increased to 50% (more or less) after 5 weeks (FIG. 13A). All lines had a similar relative water content of 92±1% and hence differences in fresh weight were primarily caused by differences in dry matter. Both HDC1-overexpressing lines showed enhanced growth, with OX2 (Ubi10) being consistently slightly bigger than OX1 (35S) plants. A positive correlation between HDC1 expression level and growth was further confirmed in hdc1-1::HDC1 complementation lines. Plant sizes and weights reflected the HDC1 protein levels in the lines (FIG. 13B). No growth phenotype has been reported for A. thaliana histone deacetylase mutants to date. We therefore re-assessed growth of hda6 knockdown (axe1-5) plants in our growth conditions. Indeed axe1-5 plants produced less fresh and dry weight than the corresponding wildtype plants (Col-0 DR5) despite slightly higher leaf number (FIG. 15). By contrast, HDA6-overexpressing plants had similar weights as wildtype plants (FIG. 10) and therefore did not phenocopy HDC1-overexpressing lines.

### Example 8

### HDC1 Alters Transcript Levels and Acetylation Status of Salt Stress-Regulated Genes

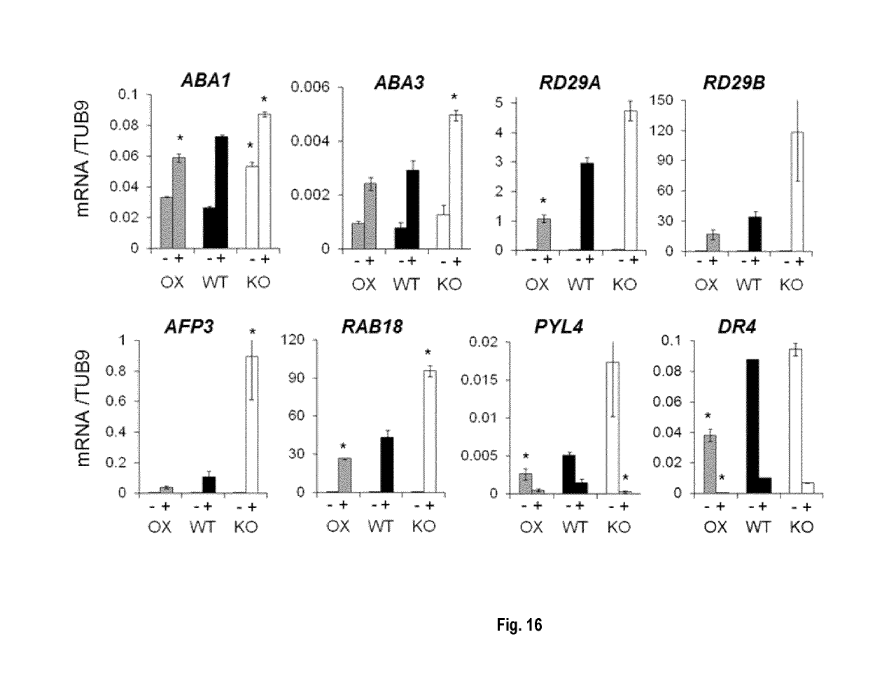

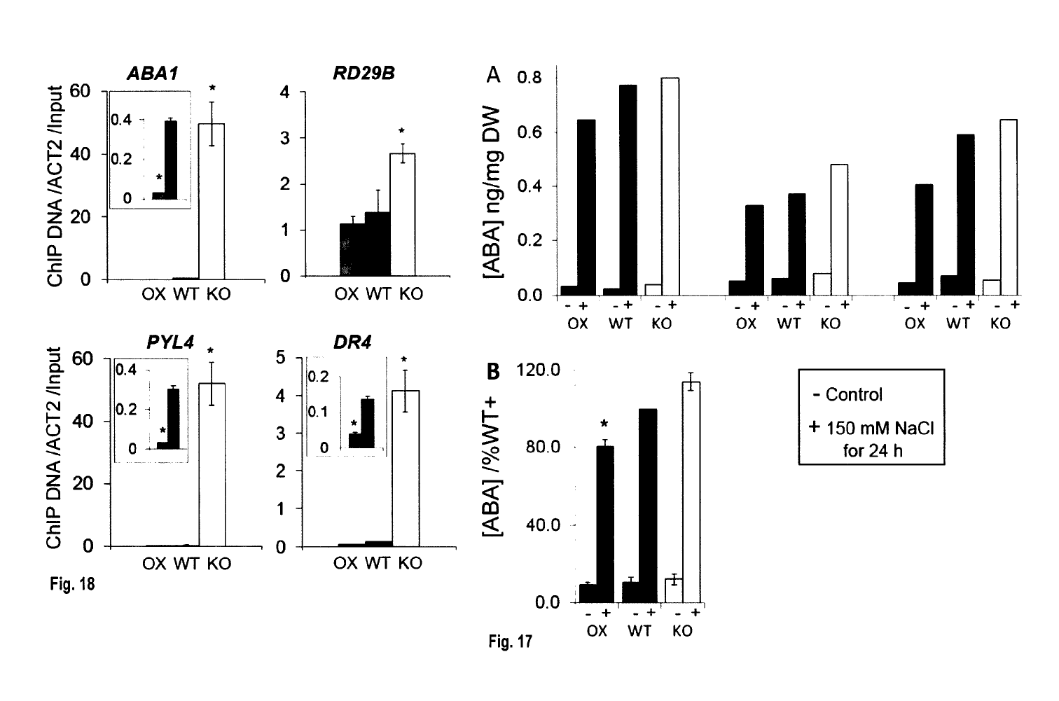

To examine a function of HDC1 in transcriptional regulation, we treated 4-weeks old hydroponically grown wildtype and mutants plants with 150 mM NaCl for 24 hours, and determined transcript levels of several known salt stress-responsive genes including ABA-biosynthesis genes ABA1 and ABA3, transcription factors Rd29A/B, dehydrin Rab18 and ABI5-binding protein AFP3 (Yamaguchi-Shinozaki and Shinozaki, 2006, supra). We found that after the salt treatment transcript levels showed a consistent profile across the lines with higher levels in hdc1-1 and/or lower levels in HDC1-OX plants than in wildtype plants (FIG. 16). In control conditions, transcript levels of the genes were similarly low in all lines apart from ABA1 transcript which was increased in hdc1-1. Shoot ABA levels confirmed that ABA biosynthesis was efficiently induced by salt in al lines but attained levels were slightly higher/lower in hdc1-1/OX lines (FIG. 17).

ABA-receptor PYL4 and of ‘drought-repressed’ gene DR4 were efficiently repressed by salt stress in all lines but higher/lower transcript levels in hdc1-1/HDC-OX plants were recorded in control conditions.

To assess whether and which of the observed transcriptional changes were a direct consequence of altered histone acetylation status, we performed anti-H3K9K14ac ChIP-qPCR on regions encompassing the start codons of the above genes. For ABA1, RD29B, PYL4 and DR4 we recovered less ChIP-DNA from HDC1-OX plants and more from ChIP-DNA hdc1-1 plants than from wildtype plants (FIG. 18). By contrast, no change was found for ABA3, suggesting that the transcriptional changes in this gene are the result of positive feedback control through ABA (Barrero et al., 2006, Plant Cell Env. 29:2000-2008). Acetylation status of other genes remain to be tested. The results identify ABA1, RD29B, PYL4 and DR4 as direct targets of HDC1-facilitated histone de-acetylation, and they provide a mechanistic explanation for the altered transcriptional responses of these genes in the mutants.

### Example 9

### The Growth-Enhancing Effect of HDC1 Overexpression is Maintained Under Water Stress

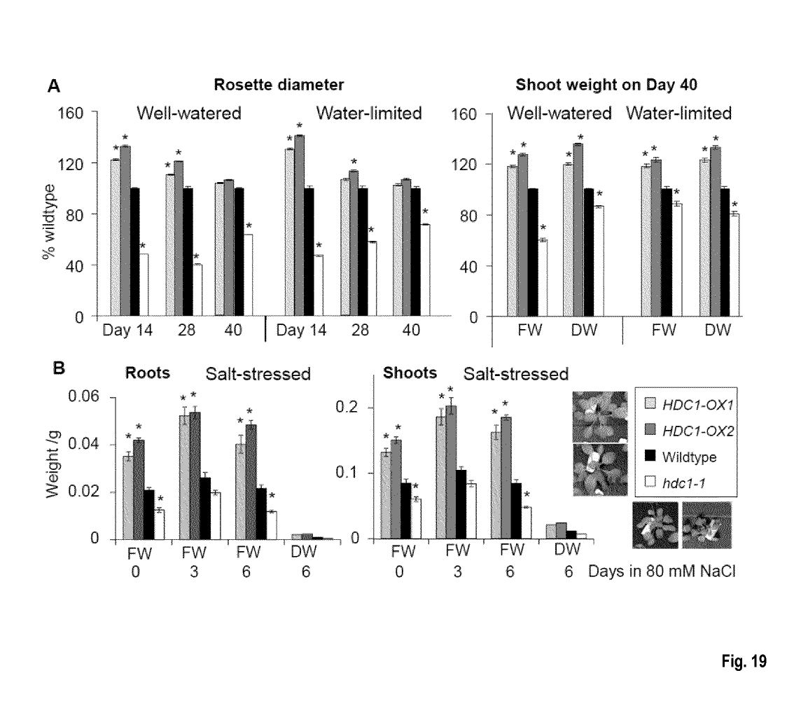

The combination of enhanced growth with lower expression of stress-inducible genes in HDC1-OX lines raised our curiosity about the net outcome of these potentially counter-productive features on plant performance under water or salt stress. We therefore subjected HDC1 mutant lines and wildtype plants to a controlled water-limiting regime in short-day conditions that started on day 14 and imposed a continuous relative soil water content of ˜50% of the control condition for the remainder of the experiment (FIG. 19A). Differences in growth between the lines were apparent in larger (HDC1-OX) and smaller (hd1-1) rosette diameters of younger plants, recorded on day 14 and 28. In older plants, rosette diameters differed less due to maximal extension of the outer leaves, but significant differences of total shoot fresh and dry weights were found when the plants were harvested on day 40 (before flowering). In well-watered conditions, shoot fresh weights were ˜20% higher in HDC1-OX plants and ˜40% lower in hdc1-1 plants than in wildtype plants. Limited water supply slowed the growth of all lines (by ˜30% on day 28 and ˜80% on day 40), yet HDC1-OX plants still produced significantly higher (˜20%) biomass than wildtype plants, and hdc1-1 knockout plants were still significantly smaller than wildtype plants (although the difference in fresh weight had narrowed to ˜10%, FIG. 19A).

In a second experiment, hydroponically grown plants were subjected for 6 days to a moderate salt stress (80 mM NaCl, FIG. 19B). The stress did not produce severe chlorosis or desiccation, but it reduced shoot water content (from 92±1% to 86±1% after 6 days) and slowed growth in all lines (compare data for control plants in FIG. 13). Under salt stress, HDC1-OX continued to produce significantly more root and shoot biomass than wildtype and hdc1-1 plants remained smaller. Thus, lower responsiveness of salt-inducible genes in HDC1-OX plants does not seem to present a disadvantage for growth under moderate salt stress.

### Example 10

### HDC1 Overexpression in Wheat: Materials and Methods

### Cloning Procedures

The 2757 bp coding sequence of the A. thaliana HDC1 gene (SEQ ID NO.: 5) was optimized for wheat codon usage (resulting in the nucleotide sequence of SEQ ID NO: 54). A BsaI site was created at the ATG and a MluI site behind the stop codon. A gel-purified BsaI-MluI fragment containing the optimized hdc1 gene was ligated between the maize ubiquitin-1 promoter PubiZm and a nos terminator in a NcoI-MluI digested vector pTCD145 that contains in addition a P35S:bar selectable marker cassette. The ligation reaction product was used to transform MC1061 bacterial cells. Antibiotic marker-resistant colonies were isolated and verified by restriction digest analysis and sequencing.

The plant transformation vector pTVE704 used for the generation of the wheat transgenics (SEQ ID NO. 55) contains two expression cassettes. The selectable marker cassette has the 35S promoter driving the Bar gene and the hdc1 cassette has the maize ubiquitin-1 promoter driving the codon optimized A. thaliana HDC1 coding sequence. The pTVE704 vector backbone is derived from pGSC1700 (Cornelissen and Vandewiele, 1989: Nuclear transcriptional activity of the tobacco plastid psbA promoter. Nucleic Acids Research, 17, 19-25).

### Plant Transformation

Plasmids were inserted by heat shock into Agrobacterium tumefaciens strain AGL1 (Lazo et al. 1991). Agrobacterium-mediated transformation of Triticum aestivum immature embryos was performed using a modification of the Rothamsted method (Wu et al. 2003: Factors influencing successful Agrobacterium-mediated genetic transformation of wheat. Plant Cell Reports, 21, 659-668). Plants were selected using media containing PPT and regenerated plantlets were transferred to the greenhouse to obtain multiple events. Single copy events were confirmed by Southern Blot analysis.

### Example 11

### Effect of HDC1 Overexpression in Wheat on Biomass

### Plant Material and Growth Conditions

To evaluate the response of wheat (Triticum aestivum) containing the HDC1 gene under drought and control conditions, several independent events of the variety Fielder transformed using Agrobacterium tumefaciens with a single copy of the HDC1 gene combined with the bar gene as a selectable marker were used.

120 seeds of each event and 30 seeds of the wild type variety Fielder were sown in zip lock bags and put in a fridge at 4° C. and a 12 h light regime. After 8 days, the seeds were sown in square 9 cm pots and put in a growth chamber with a 16 h light regime (app. 250 par), with a day temperature of 20-22° C. and a night temperature of 14-16° C.

### Selection of Plant Material

At 1-2 leaf stage, the plants for each event were sampled for cRT-PCR of bar and taqman for presence/absence of the HDC1 gene. For each event, homozygous plants were selected to be used for the experiment.

### Treatment

All plants were treated identically to normal watering until 19 days after sowing, when two treatments were imposed. Normal watering (“control”) maintained the optimal watering, whilst a restricted watering regime to impose drought stress (“drought”). Soil Water Capacity (SWC) and Soil Retention Capacity (SRC) of the used soil were determined at the start of the experiment. These data were used to determine the target weights of the pots for each treatment. The pots with normal watering were kept at 50% SRC, the pots used in the restricted watering regime were kept at 40% SRC. All pots were weighed on daily basis and if needed, water was added until the target weight was reached. The plants were ordered in a randomized block design with 5 repetitions for each homozygous event and the wild type variety Fielder as control.

### Sampling for Fresh Weight Determination

After 14 days of treatment, 33 days after sowing, all plants were harvested to determine fresh weight.

### Data Analysis

All data was recorded using Excel. Data was analyzed using the statistical programming language R. To determine the effects between the homozygous genotypes and the wild type control, a two way ANOVA was used.

### Results

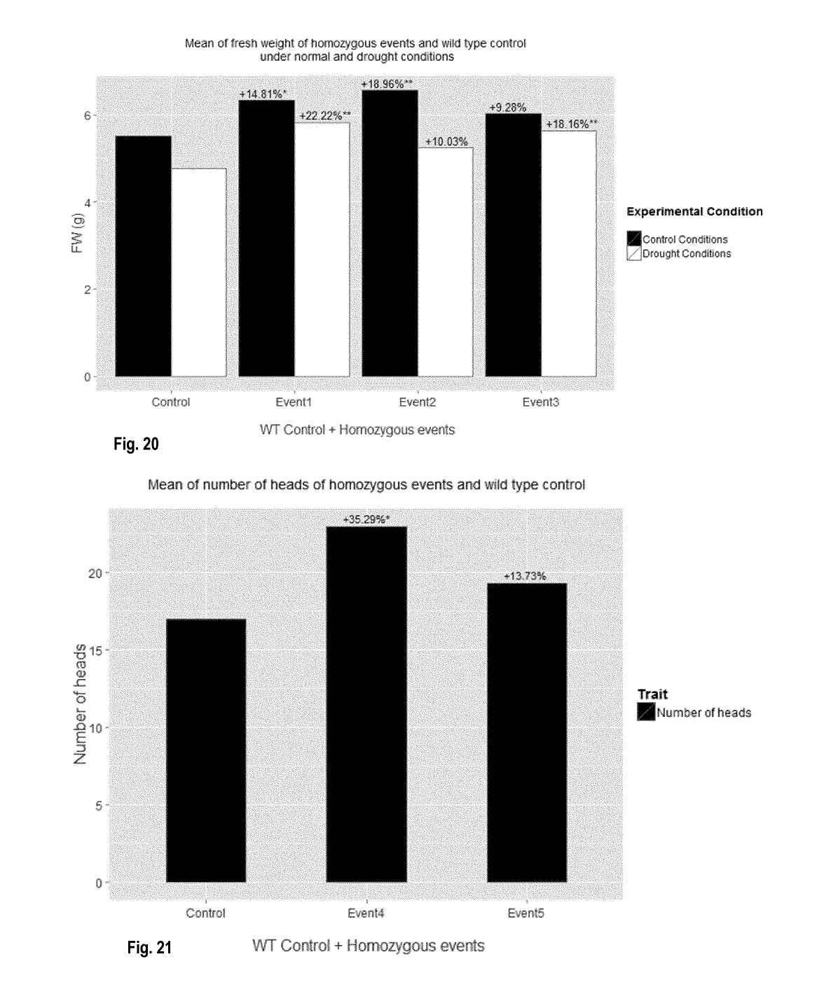

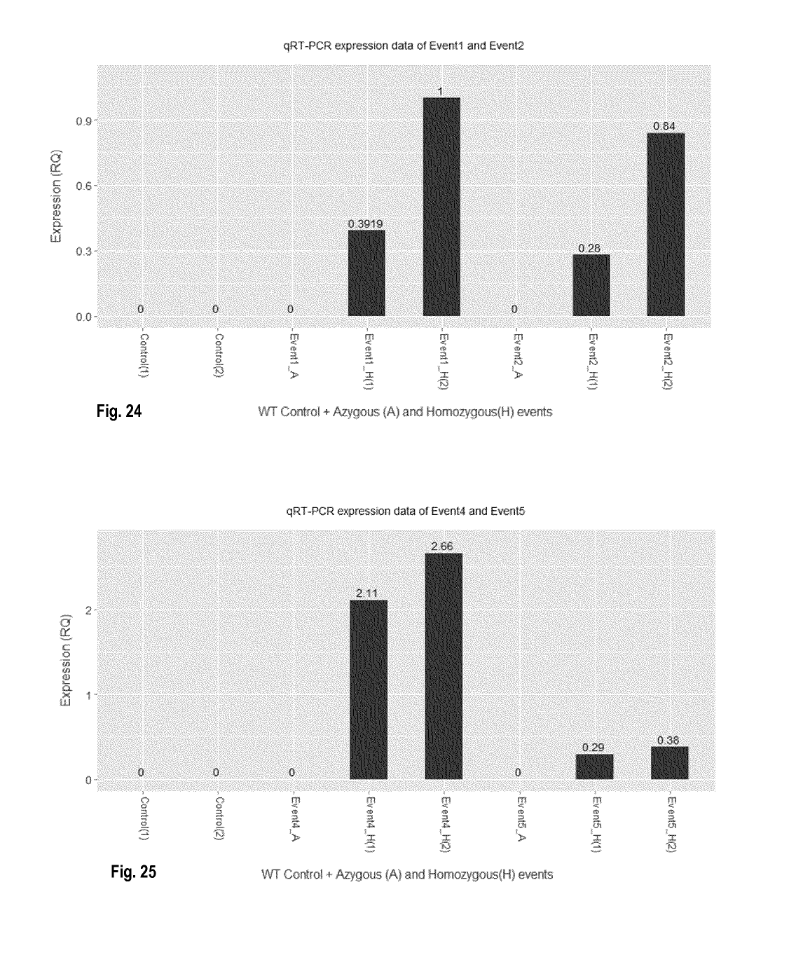

Whilst no expression of HDC1 was detected in wild type control or azygous plants, a strong overexpression of HDC1 was detected in event#1 and event#2 (FIG. 24). Expression was not determined in event#3 since the left border of the T-DNA was not found to be intact. In the biomass experiment, 3 independent events (#1, #2 and #3) performed better under drought, as well as under control conditions (FIG. 20). For those events, there was an increase of 10-20% increase in biomass (fresh weight) under drought conditions in comparison to the wild type control. The events showed an increase of 9-19% in biomass (fresh weight) under control conditions in comparison to the wild type control.

### Example 12

### Effect of HDC1 Overexpression in Wheat on Yield

### Plant Material and Growth Conditions

To evaluate the response of wheat (Triticum aestivum) containing the HDC1 gene under control conditions, several independent events of the variety Fielder transformed using Agrobacterium tumefaciens with a single copy of the HDC1 gene combined with the bar gene as a selectable marker were used. Integrity of the construct was confirmed using left border/right border analysis with PCR, all events with a border that was not intact were excluded from the experiment.

50 seeds of each event and 30 seeds of the wild type variety Fielder were sown in zip lock bags and put in a fridge at 4° C. and a 12 h light regime. After 8 days, the seeds were sown in square 9 cm pots and were put in a greenhouse compartment with a 16 h light regime (app. 250 par), with a day temperature of 20-22° C. and a night temperature of 14-16° C. After selection, the plants were transplanted in 17 cm pots, and were watered with drip irrigation. The plants were grown until full maturity.

### Selection of Plant Material

At 1-2 leaf stage, the plants were sampled for cRT-PCR of bar and taqman for presence/absence of the HDC1 genes. Of each line, 3 homozygous plants were selected to be grown under normal watering conditions (“control”).

### Yield Traits Observations

The following traits were analyzed during the seed production:

Number of tillers and number of heads

Number of seeds per plant

Yield in gram per plant

### Data Analysis

All data was recorded using Excel. Data was analyzed using the statistical programming language R. To determine the effects between the homozygous genotypes and the wild types, a two way ANOVA was used.

### Results

Whilst no expression of HDC1 was detected in wildtype control or azygous plants, a strong overexpression of

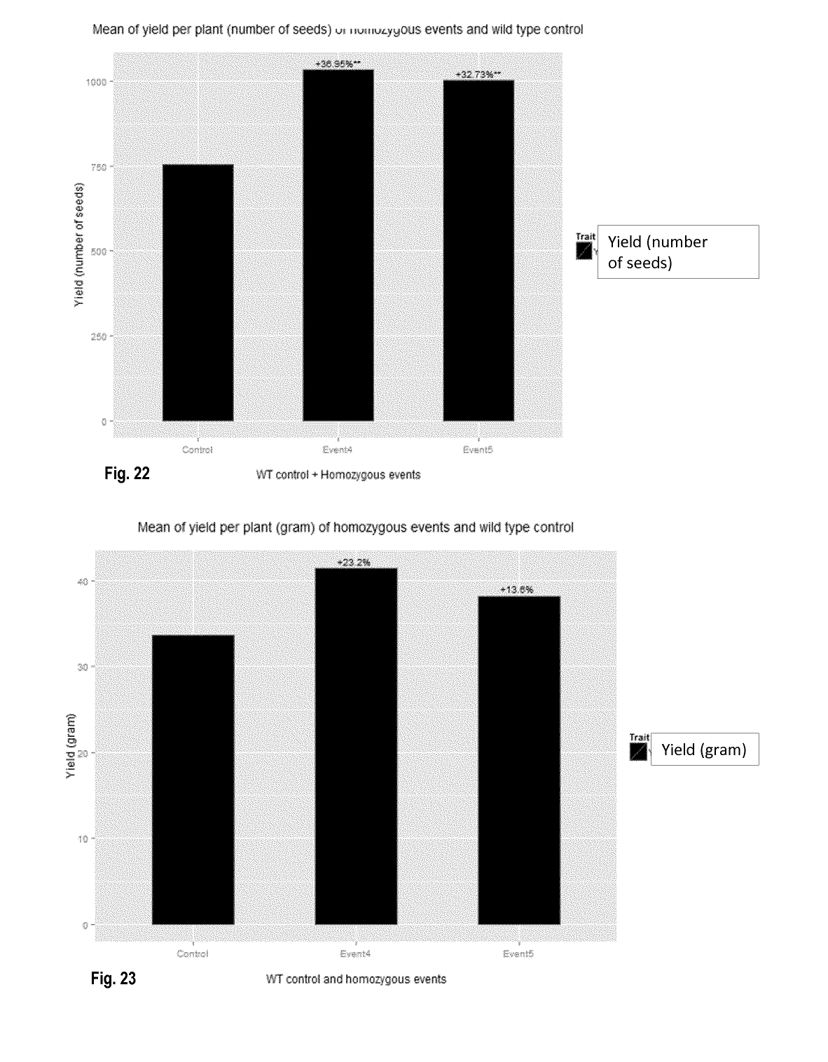

HDC1 was detected in event#4 and event#5 (FIG. 25). Two of the studied events showed an increase of 14% (Event5) and 35% (Event4) in comparison to the wild type control in the number of heads (FIG. 21). These events showed an increase of 14% (Event5) and 23% (Event4) in yield (gram) in comparison to the wild type control (FIG. 23) and an increase of 33% (Event5) and 37% (Event4) in yield (number of seeds) in comparison to the wild type control (FIG. 22).

### Example 13

### HDC1 Overexpression in Crop Plants

HDC1 overexpression constructs are transformed into crop plants other than wheat according to standard methods known in the art and overexpression is confirmed by RT-PCR, Northern or western blotting. Biomass (of vegetative tissue and seeds) of plants overexpressing HDC1 grown under various stress conditions as described above (e.g. water limiting conditions, salt stress, osmotic stress) or grown under non-stress condition are compared to wt plants grown under the same conditions. An increased biomass is observed in HDC1-overexpression plants compared to wt, both under stress and under non-stress conditions.

Seeds of the above plants overexpressing HDC1 are subjected to ABA, osmotic stress and/or histone deacetylase inhibitors, and germination was compared to seeds of control plants as described above. Germination of the HDC1 overexpressing seeds was less inhibited by the above treatment compared to wt seeds.

Also, flowering time, seed yield and plant height of HDC1-overexpressing crop plants is compared to that of wt plants. Overexpressing plants display an earlier flowering time than wt plants, an increased seed yield and increased plant height as compared to wt plants.

1. A method for increasing tolerance of a plant, plant part, plant organ or plant cell to stress conditions; or for reducing ABA sensitivity of a plant, plant part, plant organ or plant cell; or for increasing biomass or yield or growth rate of a plant, plant organ or plant part; or for accelerating flowering time of a plant; comprising the step of
a. increasing the expression and/or activity of a protein having the activity of the protein with the amino acid sequence of SEQ ID NO. 6, in said plant, plant part, plant organ or plant cell.

2. The method according to claim 1, wherein said stress condition is a moderate stress condition.

3. The method according to claim 1 or 2, wherein said increasing the expression and/or activity of a protein having the activity of the protein with the amino acid sequence of SEQ ID NO. 6 comprises expressing in said plant cell, plant part, plant organ or plant a chimeric gene comprising the following operably linked elements:
i. A plant-expressible promoter
ii. A nucleic acid which when transcribed results in an increased activity and/or expression of a protein having the activity of the protein encoded by SEQ ID NO. 6
iii. Optionally, a 3′ end region involved in transcription termination and polyadenylation functional in plants

4. The method according to claim 3, wherein said nucleic acid encodes a protein having the activity of the protein with the amino acid sequence of SEQ ID NO. 6.

5. The method according to claim 3 or 4, wherein said nucleic acid comprises a nucleic acid sequence encoding a protein having at least 70% sequence identity to SEQ ID NO. 6, SEQ ID NO. 8, SEQ ID NO. 10, SEQ ID NO. 12, SEQ ID NO. 14, SEQ ID NO. 16, SEQ ID NO. 18, SEQ ID NO. 20, SEQ ID NO. 22, SEQ ID NO. 24, SEQ ID NO. 26, SEQ ID NO. 28, SEQ ID NO. 30, SEQ ID NO. 32, SEQ ID NO. 34, SEQ ID NO. 36, SEQ ID NO. 38, SEQ ID NO. 40 or SEQ ID NO. 41, or a nucleic acid sequence having at least 70% sequence identity to SEQ ID NO. 5, SEQ ID NO. 7, SEQ ID NO. 9, SEQ ID NO. 11, SEQ ID NO. 13, SEQ ID NO. 15, SEQ ID NO. 17, SEQ ID NO. 19, SEQ ID NO. 21, SEQ ID NO. 23, SEQ ID NO. 25, SEQ ID NO. 27, SEQ ID NO. 29, SEQ ID NO. 31, SEQ ID NO. 33, SEQ ID NO. 35, SEQ ID NO. 37 or SEQ ID NO. 39.

6. The method according to any one of claims 3-5, wherein said promoter is a constitutive promoter or an inducible promoter.

7. The method according to any one of claims 1-6, wherein said plant is selected from wheat, oilseed rape, lettuce, tobacco, cotton, corn, rice, vegetable plants, carrot, cucumber, leek, pea, melon, potato, tomato, sorghum, rye, oat, sugarcane, peanut, flax, bean, sugar beets, soy bean, sunflower, ornamental plants.

8. The method according to any one of claims 1-7, wherein said stress condition is selected from drought stress, salt stress, low nutrient levels, high light stress and oxidative stress.

9. A method for enhancing survival of a plant, plant part, plant organ or plant cell under severe stress conditions, or for enhancing recovery after severe stress of a plant, plant part, plant organ or plant cell, or for delaying the flowering time of a plant, comprising the step of:
a. decreasing the expression and/or activity of a protein having the activity of the protein encoded by SEQ ID NO.6 in said plant, plant part, plant organ or plant cell.

10. The method of claim 9, wherein said reducing the expression and/or activity comprises expressing in said plant cell, plant part, plant organ or plant a chimeric gene comprising the following operably linked elements:
i. A plant-expressible promoter
ii. A nucleic acid which when transcribed results in a decreased activity and/or expression of a protein having the activity of the protein encoded by SEQ ID NO. 6
iii. Optionally, a 3′ end region involved in transcription termination and polyadenylation functional in plants

11. The method of claim 10, wherein said nucleic acid when transcribed yields an HDC1 inhibitory RNA molecule.

12. The method of claim 11, wherein said promoter is an inducible promoter.

13. A chimeric gene as described in any one of claim 3-6 or 10-12.

14. A plant, plant part, plant organ, plant cell or seed comprising the chimeric gene of claim 13.

15. The plant, plant part, plant organ, plant cell or seed of claim 14, which is oilseed rape, lettuce, tobacco, cotton, corn, rice, wheat, vegetable plants, carrot, cucumber, leek, pea, melon, potato, tomato, sorghum, rye, oat, sugarcane, peanut, flax, bean, sugar beets, soya, sunflower, ornamental plants.

16. Method for reducing yield penalty of a plant under stress conditions comprising expressing in said plant a chimeric gene as described in any one of claims 3-6.

17. A method for producing a plant with increased tolerance to stress conditions, or a plant with reduced ABA sensitivity, or a plant with increased biomass or yield or growth rate, or a plant with an earlier flowering time, comprising the steps of:
a. Introducing into a cell of a plant a chimeric gene as described in any one of claims 3-6 to generate a transgenic cell; and
b. Generating a plant, plant part, plant organ from said transgenic plant cell expressing said chimeric gene.

18. A method for modulating histone acetylation in a cell, comprising the step of modulating the expression and/or activity of a protein having the activity of the protein encoded by SEQ ID NO.6 in said cell, wherein increasing the expression and/or activity of said protein inhibits histone acetylation and decreasing the expression and/or activity of said protein enhances histone acetylation.

19. Use of a chimeric gene as described in any one of claims 3-6 to increase the tolerance of a plant, plant part, plant organ or plant cell to stress conditions; or to reduce ABA sensitivity of a plant, plant part, plant organ or plant cell; or to increase biomass or yield or growth rate of a plant, plant organ or plant part; or to accelerate flowering time of a plant.

20. Use of the plant of claim 14 or 15, to produce seed comprising the chimeric gene of claim 13.

21. Use of the plant of claim 14 or 15 comprising a chimeric gene as described in any one of claims 3-6 to produce a population of plants with increased tolerance to stress conditions, preferably moderate stress conditions or with reduced ABA sensitivity, or with increased biomass or yield or growth rate, or with an accelerated flowering time.

22. A protein having the activity of the protein with the amino acid sequence of SEQ ID NO. 6.

23. The protein of claim 22, having at least 70% sequence identity to SEQ ID NO. 6, SEQ ID NO. 8, SEQ ID NO. 10, SEQ ID NO. 12, SEQ ID NO. 14, SEQ ID NO. 16, SEQ ID NO. 18, SEQ ID NO. 20, SEQ ID NO. 22, SEQ ID NO. 24, SEQ ID NO. 26, SEQ ID NO. 28, SEQ ID NO. 30, SEQ ID NO. 32, SEQ ID NO. 34, SEQ ID NO. 36, SEQ ID NO. 38, SEQ ID NO. 40 or SEQ ID NO. 41.

24. A nucleic acid encoding the protein of claim 22 or 23.

25. The nucleic acid of claim 24, having at least 70% sequence identity to SEQ ID NO. 5, SEQ ID NO. 7, SEQ ID NO. 9, SEQ ID NO. 11, SEQ ID NO. 13, SEQ ID NO. 15, SEQ ID NO. 17, SEQ ID NO. 19, SEQ ID NO. 21, SEQ ID NO. 23, SEQ ID NO. 25, SEQ ID NO. 27, SEQ ID NO. 29, SEQ ID NO. 31, SEQ ID NO. 33, SEQ ID NO. 35, SEQ ID NO. 37 and SEQ ID NO. 39.


SEQUENCE LISTING (55 sequences)

SEQ ID NO: 1 - DNA, 647 bp, Arabidopsis thaliana
tatataaataccaaggtgatatgactccttccttcgatttatttatttattattttatttcgtctcagtgaatttaatgagctctgttttccgttgactttttattgtactgtataaaaaaaattaaaaacgacaaaatctatatcctatgaacaattcaattaatagaaagttttatggaaaaagtgagagattgaataagtatgagggcataacggcaataaataaaacctaaattgtggagacttgtaagagcacgacggtctgtgacaagaagcaaatattaacgcgaaaaataaacatttgtccaaaataaagtagcaaaccaaggagaacggaaaataaattagactcatcagagaaactcagagagaggcaaaagtccgaatccagtttgccatttattacttcccggcggcaaaatccaaaagggtttgcttcttcgtgctctgcttcagtttcaattggtaaaagaaatatcctttttaaaaaaatcttcggctctgtgttcattttagggattcaatgtttagtctggtgattcaaattctgtgttttgctctaggttgtgtatgaattaagtgcaattctatctgttgcagcagtgaatttctgggttattgaatttgggagtgatgagtggtgtt

SEQ ID NO: 2 - DNA, 12856 bp, artificial
  vector
  misc_feature (10087)..(12843): inverse complement of HDC1 coding region
ttgtacaaacttgtttgatagcttggcgcgcctcgagggggggcccggtacccggggatcctctagagtcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacctgcaggcacgccaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctccaccgcggtgggcggccgctctagaactagttaattaaggaattatcgaaccactttgtacaagaaagctgggtttagttgggggagagaaaatgaacacgagcaagagtgtactcttttccagcaatccaaacaccagtttgtgaccactgtacatcttcccaatcaagattctcctccaacacctcgatatgatctgctgggagtggaaacccgatagaccgcataagcttctgtgggagaggtttcttacatcgtgaccagcggaaaacatcaattaaactgttgtctgaatctgttttgtcaccgtttgtcagatggttctgtgacttgttattattattatctgtctccatagcttcatgtgatgattgttgttgtgaggcttggattgctttgatggtcttctctcctgcgaaacagagctcatacctgcatgaatgagtttctaagtacaaaacttcccctttcttcaagcgggcagaggtgaaaagaggcttcttgagacctttatcagcaacaatgcttatgctatatttaatccaaggttcattgcagagattgtattgtattgtgacttctcgtacaaacctttgttgccgcagagcattcgaagctgcagctctggtggtcatagatctttcaacagccattggtgcaagagttggctccacagttgaggagtgtgtaagggaaggttccagttcaatagtcccacctcctttcttcagtatatagcaccgctcaactctataactgcatccgattccagctccccatgctcgagaacggacattgttccttagcttggaggtgtagtaatcttgtgacggcaagactctaatagtagtgcgcagctcttgcattgtcggtggaggaggagaagctgtgggacgacagtaacctgtatgcatgagaacagcaacaagatcggaatcgtctgtgtatatatctgttccccatagttggccacctcttacttggcgatttgtagcagtaacatgctcagctggaatcctaacttcaagagtggggccattattagcgaaatcaccgcttttatcaggatgagacaaatcatattctttccacaacttaatcagttcttgcatacattcgccaactttgtaaacaacaatcgacacctctgacttgccttgtactccttcgttgtcctgactccgtgagcggacattgtcgcgattagtggtttgtgggctgcctctcggtctcagcgctctcttcctctgctgaaccccataattgaaggcatccttttccctctcggtagctccttcaccctctaaacacccatcttcagattctttttcactgattctgctgcgcttttcagctctttctgcctctgaatcaccatccctctctctccttttttcctttgtttctctttcgtctttttcacaattatccggttcgttctgcttcttctgctctggtgccacatattcctgctctgagggtttggcagatgcttctcccagctctttctcgttctgcgagatctctttctcagcaccagtccttggctctcttttgatatgatctttatctttctctttatttctttctcgatctttctgctccatcctctcccgttcccacctatcggattccctttcttctcttccaatctctttgggttcactcatgacactaccaacaagcacagatactcgacggtcatttctatccttgtctcggtccccccattctcgatgctttaactcttttcttttcttatctttttccttaaatctatcttcgtttttggcatcaaccttgttttctccgacggtttcacgtccttccaaatgagacccctccacaggcgcagagagatctttaggcccaacctcagttgggccttgcggattaccgcgggatacaacccacgggtccacatttgcagtcgaaccttcagcaactctcttccctctatggtaatccttctgctctttccaagccaagtgagcatgcccttccttttccatcttaatctcccccttttgttcattataattttgattctccctatcaaattttgtatccctagtatagctaccggcattacttttcccgctaaaatcatcccctggacgctcaaacttggcatccctgtcactcttaggaccctgaatctccctcttagtctcaccatacatctctctcccatcattcctagtatagcttccggtattaccttttccgctaaagtcatctactgatctctcaaatttcacatctctgtctcccttaggaccctgaatctccctctttgtctcaccatatatctctctcccatcactcctattttctctactctcaaccctaatttccctgccatccttagcaccatctctcggctccatcggcacaggggcgtgagtcaaatgaggatcactagaagaaacagttgtgggcagcgacggagaccgatagacaagaggcagaggagagcgtctctctccatctctaggctcgcttctcgcaaccttaaccaccgttctagattcaacctcataaggagcagaagcagaagcagcagcagcaagtggtgaatgggagtgagaatgagaatgagggtgaggaagcgcctggaggtgaggttgaggctgagattgagattgagattggggatgctgatggggctgttgatggttatgatgaacctgagccggtggtggcgtcacaggctgatgcggcgatttagggtaagatccagaatcctcgtgagggtattttgctactgatgaagaagaagatggatgagtaacaccctcttcgtgagatctctttggaacaccactcattaagcctgctttt

SEQ ID NO: 3 - DNA, 11922 bp, artificial
  vector
  misc_feature (9155)..(11911): HDC1 region
ttgtacaaagtggtgatgggacgtccgcggagatctacgcgtgtcgactcgagatatccaactagtttataagcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgcaggcatgcgacgtcgggccctctagaggatccccgggtaccgcgaattatcgatcatgagcggagaattaagggagtcacgttatgacccccgccgatgacgcgggacaagccgttttacgtttggaactgacagaaccgcaacgttgaaggagccactgagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaactcgatcgaggggatctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggtccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtgccgccccgtccggtcctgcccgtcaccgaaatctgatgacccctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaccggcatgcaagctgataattcaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagtcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccctaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttaaagtactttaaagtactttaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtagattaatctgagtttttctgattaacactcgagtgcgggatcctctaagggcccatcacaagtttgtacaaaaaagcaggcttaatgagtggtgttccaaagagatctcacgaagagggtgttactcatccatcttcttcttcatcagtagcaaaataccctcacgaggattctggatcttaccctaaatcgccgcatcagcctgtgacgccaccaccggctcaggttcatcataaccatcaacagccccatcagcatccccaatctcaatctcaatctcagcctcaacctcacctccaggcgcttcctcaccctcattctcattctcactcccattcaccacttgctgctgctgcttctgcttctgctccttatgaggttgaatctagaacggtggttaaggttgcgagaagcgagcctagagatggagagagacgctctcctctgcctcttgtctatcggtctccgtcgctgcccacaactgtttcttctagtgatcctcatttgactcacgcccctgtgccgatggagccgagagatggtgctaaggatggcagggaaattagggttgagagtagagaaaataggagtgatgggagagagatatatggtgagacaaagagggagattcagggtcctaagggagacagagatgtgaaatttgagagatcagtagatgactttagcggaaaaggtaataccggaagctatactaggaatgatgggagagagatgtatggtgagactaagagggagattcagggtcctaagagtgacagggatgccaagtttgagcgtccaggggatgattttagcgggaaaagtaatgccggtagctatactagggatacaaaatttgatagggagaatcaaaattataatgaacaaaagggggagattaagatggaaaaggaagggcatgctcacttggcttggaaagagcagaaggattaccatagagggaagagagttgctgaaggttcgactgcaaatgtggacccgtgggttgtatcccgcggtaatccgcaaggcccaactgaggttgggcctaaagatctctctgcgcctgtggaggggtctcatttggaaggacgtgaaaccgtcggagaaaacaaggttgatgccaaaaacgaagatagatttaaggaaaaagataagaaaagaaaagagttaaagcatcgagaatggggggaccgagacaaggatagaaatgaccgtcgagtatctgtgcttgttggtagtgtcatgagtgaacccaaagagattggaagagaagaaagggaatccgataggtgggaacgggagaggatggagcagaaagatcgagaaagaaataaagagaaagataaagatcatatcaaaagagagccaaggactggtgctgagaaagagatctcgcagaacgagaaagagctgggagaagcatctgccaaaccctcagagcaggaatatgtggcaccagagcagaagaagcagaacgaaccggataattgtgaaaaagacgaaagagaaacaaaggaaaaaaggagagagagggatggtgattcagaggcagaaagagctgaaaagcgcagcagaatcagtgaaaaagaatctgaagatgggtgtttagagggtgaaggagctaccgagagggaaaaggatgccttcaattatggggttcagcagaggaagagagcgctgagaccgagaggcagcccacaaaccactaatcgcgacaatgtccgctcacggagtcaggacaacgaaggagtacaaggcaagtcagaggtgtcgattgttgtttacaaagttggcgaatgtatgcaagaactgattaagttgtggaaagaatatgatttgtctcatcctgataaaagcggtgatttcgctaataatggccccactcttgaagttaggattccagctgagcatgttactgctacaaatcgccaagtaagaggtggccaactatggggaacagatatatacacagacgattccgatcttgttgctgttctcatgcatacaggttactgtcgtcccacagcttctcctcctccaccgacaatgcaagagctgcgcactactattagagtcttgccgtcacaagattactacacctccaagctaaggaacaatgtccgttctcgagcatggggagctggaatcggatgcagttatagagttgagcggtgctatatactgaagaaaggaggtgggactattgaactggaaccttcccttacacactcctcaactgtggagccaactcttgcaccaatggctgttgaaagatctatgaccaccagagctgcagcttcgaatgctctgcggcaacaaaggtttgtacgagaagtcacaatacaatacaatctctgcaatgaaccttggattaaatatagcataagcattgttgctgataaaggtctcaagaagcctcttttcacctctgcccgcttgaagaaaggggaagttttgtacttagaaactcattcatgcaggtatgagctctgtttcgcaggagagaagaccatcaaagcaatccaagcctcacaacaacaatcatcacatgaagctatggagacagataataataataacaagtcacagaaccatctgacaaacggtgacaaaacagattcagacaacagtttaattgatgttttccgctggtcacgatgtaagaaacctctcccacagaagcttatgcggtctatcgggtttccactcccagcagatcatatcgaggtgttggaggagaatcttgattgggaagatgtacagtggtcacaaactggtgtttggattgctggaaaagagtacactcttgctcgtgttcattttctctcccccaactaaacccagctttc

SEQ ID NO: 4 - protein, 294 aa, Saccharomyces cerevisiae
MSVSEQDPNRAYRETQSQIYKLQETLLNSARTKNKQEEGQESNTHSFPEQYMHYQNGRNSAYDLPNVSSQSVLAFTEKHYPNKLKNLGTLYYNRFKEGSFDEDSTSYSDRHSFPYNLYDNTLPPPFLPAIGIQNINNIATLKITYEDIQASFNNIESPRKRNNEIWGCDIYSDDSDPILVLRHCGFKIGAPSGGSFHKLRRTPVNVTNQDNVTGNLPLLEGTPFDLEVELLFLPTLQKYPSVKRFDITSREWGSEATVIHDGLSYGIYSIVIKQRLDRDKPHEPNGYIKNLKWT

SEQ ID NO: 5 - DNA, 2757 bp, Arabidopsis thaliana
  CDS (1)..(2757)
atgagtggtgttccaaagagatctcacgaagagggtgttactcatccatcttcttcttcatcagtagcaaaataccctcacgaggattctggatcttaccctaaatcgccgcatcagcctgtgacgccaccaccggctcaggttcatcataaccatcaacagccccatcagcatccccaatctcaatctcaatctcagcctcaacctcacctccaggcgcttcctcaccctcattctcattctcactcccattcaccacttgctgctgctgcttctgcttctgctccttatgaggttgaatctagaacggtggttaaggttgcgagaagcgagcctagagatggagagagacgctctcctctgcctcttgtctatcggtctccgtcgctgcccacaactgtttcttctagtgatcctcatttgactcacgcccctgtgccgatggagccgagagatggtgctaaggatggcagggaaattagggttgagagtagagaaaataggagtgatgggagagagatatatggtgagacaaagagggagattcagggtcctaagggagacagagatgtgaaatttgagagatcagtagatgactttagcggaaaaggtaataccggaagctatactaggaatgatgggagagagatgtatggtgagactaagagggagattcagggtcctaagagtgacagggatgccaagtttgagcgtccaggggatgattttagcgggaaaagtaatgccggtagctatactagggatacaaaatttgatagggagaatcaaaattataatgaacaaaagggggagattaagatggaaaaggaagggcatgctcacttggcttggaaagagcagaaggattaccatagagggaagagagttgctgaaggttcgactgcaaatgtggacccgtgggttgtatcccgcggtaatccgcaaggcccaactgaggttgggcctaaagatctctctgcgcctgtggaggggtctcatttggaaggacgtgaaaccgtcggagaaaacaaggttgatgccaaaaacgaagatagatttaaggaaaaagataagaaaagaaaagagttaaagcatcgagaatggggggaccgagacaaggatagaaatgaccgtcgagtatctgtgcttgttggtagtgtcatgagtgaacccaaagagattggaagagaagaaagggaatccgataggtgggaacgggagaggatggagcagaaagatcgagaaagaaataaagagaaagataaagatcatatcaaaagagagccaaggactggtgctgagaaagagatctcgcagaacgagaaagagctgggagaagcatctgccaaaccctcagagcaggaatatgtggcaccagagcagaagaagcagaacgaaccggataattgtgaaaaagacgaaagagaaacaaaggaaaaaaggagagagagggatggtgattcagaggcagaaagagctgaaaagcgcagcagaatcagtgaaaaagaatctgaagatgggtgtttagagggtgaaggagctaccgagagggaaaaggatgccttcaattatggggttcagcagaggaagagagcgctgagaccgagaggcagcccacaaaccactaatcgcgacaatgtccgctcacggagtcaggacaacgaaggagtacaaggcaagtcagaggtgtcgattgttgtttacaaagttggcgaatgtatgcaagaactgattaagttgtggaaagaatatgatttgtctcatcctgataaaagcggtgatttcgctaataatggccccactcttgaagttaggattccagctgagcatgttactgctacaaatcgccaagtaagaggtggccaactatggggaacagatatatacacagacgattccgatcttgttgctgttctcatgcatacaggttactgtcgtcccacagcttctcctcctccaccgacaatgcaagagctgcgcactactattagagtcttgccgtcacaagattactacacctccaagctaaggaacaatgtccgttctcgagcatggggagctggaatcggatgcagttatagagttgagcggtgctatatactgaagaaaggaggtgggactattgaactggaaccttcccttacacactcctcaactgtggagccaactcttgcaccaatggctgttgaaagatctatgaccaccagagctgcagcttcgaatgctctgcggcaacaaaggtttgtacgagaagtcacaatacaatacaatctctgcaatgaaccttggattaaatatagcataagcattgttgctgataaaggtctcaagaagcctcttttcacctctgcccgcttgaagaaaggggaagttttgtacttagaaactcattcatgcaggtatgagctctgtttcgcaggagagaagaccatcaaagcaatccaagcctcacaacaacaatcatcacatgaagctatggagacagataataataataacaagtcacagaaccatctgacaaacggtgacaaaacagattcagacaacagtttaattgatgttttccgctggtcacgatgtaagaaacctctcccacagaagcttatgcggtctatcgggtttccactcccagcagatcatatcgaggtgttggaggagaatcttgattgggaagatgtacagtggtcacaaactggtgtttggattgctggaaaagagtacactcttgctcgtgttcattttctctcccccaactaa

SEQ ID NO: 6 - protein, 918 aa, Arabidopsis thaliana
MSGVPKRSHEEGVTHPSSSSSVAKYPHEDSGSYPKSPHQPVTPPPAQVHHNHQQPHQHPQSQSQSQPQPHLQALPHPHSHSHSHSPLAAAASASAPYEVESRTVVKVARSEPRDGERRSPLPLVYRSPSLPTTVSSSDPHLTHAPVPMEPRDGAKDGREIRVESRENRSDGREIYGETKREIQGPKGDRDVKFERSVDDFSGKGNTGSYTRNDGREMYGETKREIQGPKSDRDAKFERPGDDFSGKSNAGSYTRDTKFDRENQNYNEQKGEIKMEKEGHAHLAWKEQKDYHRGKRVAEGSTANVDPWVVSRGNPQGPTEVGPKDLSAPVEGSHLEGRETVGENKVDAKNEDRFKEKDKKRKELKHREWGDRDKDRNDRRVSVLVGSVMSEPKEIGREERESDRWERERMEQKDRERNKEKDKDHIKREPRTGAEKEISQNEKELGEASAKPSEQEYVAPEQKKQNEPDNCEKDERETKEKRRERDGDSEAERAEKRSRISEKESEDGCLEGEGATEREKDAFNYGVQQRKRALRPRGSPQTTNRDNVRSRSQDNEGVQGKSEVSIVVYKVGECMQELIKLWKEYDLSHPDKSGDFANNGPTLEVRIPAEHVTATNRQVRGGQLWGTDIYTDDSDLVAVLMHTGYCRPTASPPPPTMQELRTTIRVLPSQDYYTSKLRNNVRSRAWGAGIGCSYRVERCYILKKGGGTIELEPSLTHSSTVEPTLAPMAVERSMTTRAAASNALRQQRFVREVTIQYNLCNEPWIKYSISIVADKGLKKPLFTSARLKKGEVLYLETHSCRYELCFAGEKTIKAIQASQQQSSHEAMETDNNNNKSQNHLTNGDKTDSDNSLIDVFRWSRCKKPLPQKLMRSIGFPLPADHIEVLEENLDWEDVQWSQTGVWIAGKEYTLARVHFLSPN

SEQ ID NO: 7 - DNA, 2751 bp, Arabidopsis lyrata
  CDS (1)..(2751)
atgagtggtgttccaaagagatctcacgaagagggtgttactcatccatcttcttcttcttcagcaccaaaataccctcacgaggattctggatcttaccctaaatcgccgcatcagcctgttacgccaccaccggctcaggttcatcatcaccatcaacaacaaccccatcagcatccccaatctcaatctcaacctcaacctcaacctcaacctcacctccacacgcttcctcatccccactctcattcaccacttgctgctgcttctgcttctgctgcttatgaggttgaatctagaacggtggttaaggttgcgagaagtgagcctagagatggagagagacgctctcctctccctcttgtctatcggtctccgtccctgcccactactgtttcttctagtgatcctcatttgactcacgcccctgtgcccatggaaccgagagaaggtactaaggatggcagggaaattagggttgagaacagagaaaataggagtgatggaagggagatttatggtgagacaaagagagagattcagggtcctaagagtgacagagatgtgaagtttgatagatcagtagacgactttagcggaaaaggtaataccggaagctattctaggaatgatgggagagagatgtatggtgagacgaagagggagattcagggtcctaagagtgacagggatgccaagtttgagcgtccaggggatgattttagcggaaaaagtaataccggtagctatacgagggatacgaaatttgatagggagaatcagaattataatgaacaaaaggcggagattaagatggaaaaggacgggcatgctcacttggcttggaaagagcagaaggattaccctagaggcaagagagttgctgaaggttcgactgcaaatgtggatccgtgggttgtatcccgcggtaatccgcaaggcccaactgaggttgagcctaaagatctctccgcgccagtggaggggccccatttagaaggacgtgaaaccgtcggagaaaacaaggttgatgcaaaaaatgaagatagatttaaggacaaagataagaaaagaaaagagttaaagcatcgagaatggggggaccgagataaggatagaaatgaccgtcgaggatccgtgcttattggtagtgtcatgagtgaacccaaagagattggaagagacgaaagagaatccgataggtgggaacgggagaggatggagcagaaagatcgagaaaggaataaagagaaagataaagatcatatcaaaagagagccaaggactggtgctgagaaagagatctcacagaacgagaaagagttgggagaagcatctgccaaaccatcagagcaggaatatgtggcaccagagcagaagaagcagaacgaaccggataattgggaaaaagacgaaagagaatcaaaggaaaaaaggagagagagggatggtgattcagaggcagaaagagctgaaaagcgcagcagaatcagtgaaaaagaatctgaagatgggtgtttggagggtgaaggagctactgagagggaaaaggatgccttcaattatggagttcagcagcggaagagagcgctgagaccgagaggcagcccacaaaccacaaaccgcgaccatgtcctctcacggagtcaggacaacgatggagtacaaggcaagtcagaggtgtcgattgttgtttacaaagttggcgaatgtatgcaagaactgattaaattgtggaaagaatatgatttgtctcatcctgataaaagcggtgattttgcaaataatggccccactcttgaagttaggattccagctgagcatgttactgctacaaatcgccaagtaagaggtggccagctatggggaacagatatatacacagacgattccgatcttgttgctgttctcatgcatacaggttactgtcgtcccacagcttctcctcctccaccgacaatgcaagagctgcgcactactattagagtcttgccgtcacaagattactacacctccaagctaaggaataatgtccgttctcgagcatggggagctggaatcggatgcagttacagagttgagcggtgctatatactgaagaaaggaggtgggactattgaactggaaccttctcttacacactcctcaactgtggagccaacacttgcaccaatggctgttgaaagatctatgaccaccagggctgcagcttcgaatgctctgcggcaacaaaggtttgtacgagaagtcacaatacaatacaatctctgcaatgaaccttggatcaaatatagcataagcattgttgctgataaaggtctcaagaagcctcttttcacctctgcccgcttgaagaaaggagaagttttgtacttagaaactcattcatgcaggtatgagctctgtttcgctggagagaaaaccatcaaagcaatccaagcgtctcaacaacaatcatcacatgaagctatggagacagataataataataacaagtcacagaaccatctgacaaacggtgacaaaacagattcagacaacagtttaatcgatgttttccgttggtcacgctgtaagaaacctctcccgcagaagcttatgcggtctatcgggattccactcccagcagatcatatcgaggtgttggaggagaatcttgattgggaagatgtacagtggtcacaaactggtgtttggattgctggaaaagagtacacacttgctcgtgttcattttctctcgcccaactaa

SEQ ID NO: 8 - protein, 916 aa, Arabidopsis lyrata
MSGVPKRSHEEGVTHPSSSSSAPKYPHEDSGSYPKSPHQPVTPPPAQVHHHHQQQPHQHPQSQSQPQPQPQPHLHTLPHPHSHSPLAAASASAAYEVESRTVVKVARSEPRDGERRSPLPLVYRSPSLPTTVSSSDPHLTHAPVPMEPREGTKDGREIRVENRENRSDGREIYGETKREIQGPKSDRDVKFDRSVDDFSGKGNTGSYSRNDGREMYGETKREIQGPKSDRDAKFERPGDDFSGKSNTGSYTRDTKFDRENQNYNEQKAEIKMEKDGHAHLAWKEQKDYPRGKRVAEGSTANVDPWVVSRGNPQGPTEVEPKDLSAPVEGPHLEGRETVGENKVDAKNEDRFKDKDKKRKELKHREWGDRDKDRNDRRGSVLIGSVMSEPKEIGRDERESDRWERERMEQKDRERNKEKDKDHIKREPRTGAEKEISQNEKELGEASAKPSEQEYVAPEQKKQNEPDNWEKDERESKEKRRERDGDSEAERAEKRSRISEKESEDGCLEGEGATEREKDAFNYGVQQRKRALRPRGSPQTTNRDHVLSRSQDNDGVQGKSEVSIVVYKVGECMQELIKLWKEYDLSHPDKSGDFANNGPTLEVRIPAEHVTATNRQVRGGQLWGTDIYTDDSDLVAVLMHTGYCRPTASPPPPTMQELRTTIRVLPSQDYYTSKLRNNVRSRAWGAGIGCSYRVERCYILKKGGGTIELEPSLTHSSTVEPTLAPMAVERSMTTRAAASNALRQQRFVREVTIQYNLCNEPWIKYSISIVADKGLKKPLFTSARLKKGEVLYLETHSCRYELCFAGEKTIKAIQASQQQSSHEAMETDNNNNKSQNHLTNGDKTDSDNSLIDVFRWSRCKKPLPQKLMRSIGIPLPADHIEVLEENLDWEDVQWSQTGVWIAGKEYTLARVHFLSPN

SEQ ID NO: 9 - DNA, 2433 bp, populus trichocarpa
  CDS (1)..(2433)
atgagtggtgctcctgttaaaagatcgcatgaagagggtagtcattcttcttctttgaaattccctcctcatgaagatacaggttcgtatcctaagctgacatcaggggtttcaaatgagttccatctaccatatgagatgggtccagatgctagggtggctaagattcccagaactgagtctcgagacgtagatagaagatcacctttgcattcgatgtatcgaatcccaccatcttcaaatgaatcacacatggattctcatttgaatgttgctcctgaaagaaggcctgaatcaagggattccaaggactgcagagactaccggattgaaaaccgtgagccaaggactgatgcaagagagatgtatggcgaggcaaagagggattcacaaagtgttaaaaatgaaaaggatgtgaggtttgatagtagaggggatgacaataaagaagtaaagcatgacagagaagctcgtattgagccgaagaatgacatgaagatagaaaaggatggttttggtcctgcaagtagtcaggtgaattggaaggaaccaaaagaataccataggggaaagagatgtttggaatctgcaggtgtacatgtggatccttggcatatatcacgtggaaattcccaaggccctgttgagattgaaaaggaagtcgtcagtatcgaggagagggatcatgccaaagttcatgaggcagttggagaaaataaagttgaattgaaaggtgacgatagatttaaagacaaggataggaagaggaaagatttgaagctccgggaatggggagacagagataaggaaagaagtgatcgaaggggaagtatgcaagtaggcaacagtattgctgagggaaaagagttggtgaaggaagagagagaaggagagaggtgggagtgggagaggaaggatctgtcaaaagacagggaaaggttaaaagagagggagaaggaccacatgaaaatagaatcaggaactggagctgaaaaggagggtttgcacaatgaaaaggagtctttggatggatctgttagaatttcagaacaggaaaatccagctttggagccaaagaaacagaaagattttgataactggaaaaatgtcgataaagaagctaaagataaaaagaaagaaagagaagccggcatagaaggagatagacctgagaagggtagcacgatgtgtgggaaagaatctgatgatggatgtgcagatggtgaaattgcaactgaaagggaaagaggagtttttaactatggagtccagcagcgcaagaggatgcttcggcctaggggcagcccgcaagtggcaaattgtgaaccctgttttaggtcccatactcaggactgtgagggatgtcaaggcaaatctgaggtatcctctgtcatttataaagttagtgaatgcatgcaagagctgataaagttatggaaggagtatgaagcatctcaatctgataaaaatagtgaaagcagccataagggccccactcttgaaattcaaataccagcagaacatattactgctacaaatcgccaagtaagaggtggacaattatgggggacagatatatacacaaatgactctgatcttgtcgctgttctcatgcatacaggctacttccgtcccactgcttctcctcctccacctgccatccaagacttatgtgctactatcagagtgttgcctccacaagatagctacatttctatgctgagaaataatgttcgttcacgtgcctggggagctggaattggttgtagctaccgtgttgagcgttgctgcatcatgaagaaaggaggtggaaccattgatcttgagccctgtcttacacatacatcagcagtggaacctactcttgctcctgtagctgttgaacggacaatgactacccgtgctgcagcttcgaatgcattgcggcaacagagatttgtacgtgaagttacaatacagtacaacctttgcaatgagccctggataaaatacagcattagtattattgctgacaagggtctgaaaaagcctctctatacttctgcacgtttgaaaaagggagaagttctatatttagaaacacattcatgcaggtacgagctctgttttacaggagagaaaatggtgaaagtgatgcaggcttctcaggtgcatgaagagacaaataagatccataatcaccacccacattcctcaaacggtgagaagcacgactttgataatgttcttattgatgtattccggtggtctcgctgtaagaaaccactaccgcagaaggtcatgcagtcagttgggatcccattgcccctggaacatgttgaggtattggaggagaatcttgactgggaggatgtgcaatggtcacaaactggtgtttggatagatggaaaagaattcacacttgctagggtgcgctttctatctccaagttag

SEQ ID NO: 10 - protein, 810 aa, populus trichocarpa
MSGAPVKRSHEEGSHSSSLKFPPHEDTGSYPKLTSGVSNEFHLPYEMGPDARVAKIPRTESRDVDRRSPLHSMYRIPPSSNESHMDSHLNVAPERRPESRDSKDCRDYRIENREPRTDAREMYGEAKRDSQSVKNEKDVRFDSRGDDNKEVKHDREARIEPKNDMKIEKDGFGPASSQVNWKEPKEYHRGKRCLESAGVHVDPWHISRGNSQGPVEIEKEVVSIEERDHAKVHEAVGENKVELKGDDRFKDKDRKRKDLKLREWGDRDKERSDRRGSMQVGNSIAEGKELVKEEREGERWEWERKDLSKDRERLKEREKDHMKIESGTGAEKEGLHNEKESLDGSVRISEQENPALEPKKQKDFDNWKNVDKEAKDKKKEREAGIEGDRPEKGSTMCGKESDDGCADGEIATERERGVFNYGVQQRKRMLRPRGSPQVANCEPCFRSHTQDCEGCQGKSEVSSVIYKVSECMQELIKLWKEYEASQSDKNSESSHKGPTLEIQIPAEHITATNRQVRGGQLWGTDIYTNDSDLVAVLMHTGYFRPTASPPPPAIQDLCATIRVLPPQDSYISMLRNNVRSRAWGAGIGCSYRVERCCIMKKGGGTIDLEPCLTHTSAVEPTLAPVAVERTMTTRAAASNALRQQRFVREVTIQYNLCNEPWIKYSISIIADKGLKKPLYTSARLKKGEVLYLETHSCRYELCFTGEKMVKVMQASQVHEETNKIHNHHPHSSNGEKHDFDNVLIDVFRWSRCKKPLPQKVMQSVGIPLPLEHVEVLEENLDWEDVQWSQTGVWIDGKEFTLARVRFLSPS

SEQ ID NO: 11 - DNA, 2466 bp, Medicago truncatula
  CDS (1)..(2466)
atgagtggtacacctaagaaatctcatgaagagtctgttcatccgtcttcaaaacacccgcatgaagacgcgggtgcgtatccaaaattggcgccgtcgtcagtttcaaatgagtatcatatgtcttatgatataggtcaggattctcgggtggtaaaagtgcctcgtgatgtggagagaagatctcctcttcattcagtgtatcggatgccgtcgtcttctagtgatcctcatgccgagcatcctgttggtcctgagaagaggttagaatcaagggaatccaaggatagtagagatatccggtttgagaatcgtgatacgaagactgagaaaaaggagatgtttggagaagtaagaaaggatcctcagagtgctaaaagtgaaaaggatgcacatgttgaaggtagaggagatgacaacaaggatgttagacatgatcgggatagtcataatgattcaaaaggtgatactaagacagaaaaagatagttttaatgcggctagcggccttcacttggattggaaagaatcagaaaaataccatagggcaaaaatatattctgatcctcctggcgcgagtttggaaccctggcctatgtcacgtgggaatacacaagcttcactcgaggttggaaaggagagttcatcagcagaacaaagggagtatggtggggaagctcgtgaagctgttggggagaacaaaattgattccaaaggcgacgatagatctaaagagaaagatagaaaaagaaaggaagtgaagcatcgggactggggggagaaggaaaaagaaagaattgatcgtagaaacaatatacaagttagcaacacgggtagtgactggaaagaatctgtgaatgatcgtagaaacaatgtacaagtaagcaatacgattggtgacggcaaagaacctctgaagcaagatagagatgttgaaaggtgggagagggagaaaaaagatcttcccaaagaaaaagaaaatttaaaagagaaggaaaaggatcagatgaagagggagtcgtggaatggagccgagaaagatgtttcaaataacgagaaggaacctgttgatggatcggctaaggttcctgaacaagaaactgtcttaccggagcagaagaaacaaaaagatgttgatagagaagctaaagacaagagaaaagaaagggaagctgatttagtaggagacaggtctgataagcgcagtaggggctttgacaaggaatcagacgatggatgtgctgatgggcaaggggcaatagaaaaggagagtgaagtctataactatagtggtcagcaccgtaagaggatacaaagatcacgggggagccctcaggtgcctaatcgggagcctcgtttcaggccccgcacccaagacaacgaagggtctcaaggtaaagttgaggtttcttatgttgtttataaagttggtgaaagcatgcaagagctgataaagttgtggacggagtatgaatcatctcaatctcaaattgaaaaaaatggtgaaagctctaaaaatggccccactctggaaattcggatatcgtccgagtatgttactgctacaaatcgccaagtcagaggtggccagctttgggggactgatgtgtacacatatgactccgatcttgttgctgttctcatgcatacaggttactgtcgcccaacagcatctccacctcctgcagccatacaagagttacgcgcaaccatacgggtgctacctccaaaagattgctatatttctacactgagaaacaatgtacgttcccgtgcttggggtgctaaaattggctgcagttatcgaatcgaacggtgttgcattgtgaagaaaggaggtggaactattgatcttgaaccttgccttacacatacatcaactattgagccgacccttgctccagtggctgtggagcggacaatgactaccagggccgcagcttcaaatgcattgcggcagcaaagatatgttcgagaagtcacgattcagtacaatctttgcaatgagccttggatcaaatatagtataagcattgtagcagacaagggtctaaaaaagccacaatacacatctgctcgattgaaaaagggagaagttttgtatttggagacgcatacgaccagatacgaactatgttttgctggagagaagttggtcaaggctacaccagcaactcaggcaaatgaatcaggcgctgagaaggctcaaaatcaccatccacattctgcaaatggtgaaaaaagtgagcctgatcatgttatgattgatgcgttccggtggtctcgttgtaagaagcctctgccacagaaattgatgcgcacgattggcatccctctgcctcttgaacatgtcgaggtgttggaggagaacttggactgggaagatatacaatggtctcaaactggtgtttggattgcaggaaaggaatatacccttgcaagggtgcatttcttgtcgatgaattaa

SEQ ID NO: 12 - protein, 821 aa, Medicago truncatula
MSGTPKKSHEESVHPSSKHPHEDAGAYPKLAPSSVSNEYHMSYDIGQDSRVVKVPRDVERRSPLHSVYRMPSSSSDPHAEHPVGPEKRLESRESKDSRDIRFENRDTKTEKKEMFGEVRKDPQSAKSEKDAHVEGRGDDNKDVRHDRDSHNDSKGDTKTEKDSFNAASGLHLDWKESEKYHRAKIYSDPPGASLEPWPMSRGNTQASLEVGKESSSAEQREYGGEAREAVGENKIDSKGDDRSKEKDRKRKEVKHRDWGEKEKERIDRRNNIQVSNTGSDWKESVNDRRNNVQVSNTIGDGKEPLKQDRDVERWEREKKDLPKEKENLKEKEKDQMKRESWNGAEKDVSNNEKEPVDGSAKVPEQETVLPEQKKQKDVDREAKDKRKEREADLVGDRSDKRSRGFDKESDDGCADGQGAIEKESEVYNYSGQHRKRIQRSRGSPQVPNREPRFRPRTQDNEGSQGKVEVSYVVYKVGESMQELIKLWTEYESSQSQIEKNGESSKNGPTLEIRISSEYVTATNRQVRGGQLWGTDVYTYDSDLVAVLMHTGYCRPTASPPPAAIQELRATIRVLPPKDCYISTLRNNVRSRAWGAKIGCSYRIERCCIVKKGGGTIDLEPCLTHTSTIEPTLAPVAVERTMTTRAAASNALRQQRYVREVTIQYNLCNEPWIKYSISIVADKGLKKPQYTSARLKKGEVLYLETHTTRYELCFAGEKLVKATPATQANESGAEKAQNHHPHSANGEKSEPDHVMIDAFRWSRCKKPLPQKLMRTIGIPLPLEHVEVLEENLDWEDIQWSQTGVWIAGKEYTLARVHFLSMN

SEQ ID NO: 13 - DNA, 2418 bp, Vitis vinifera
  CDS (1)..(2418)
atgagtggtgttcccaagaggcctcacgatgaggtcggcggtggaagcggcggtgctgctgctgctgctgctgctgctgggcattcctccggtgcttctaagtatccgcatgaagattccggcaatgcatttgctgggaaattgaacccatcgtcgtcttcagcaccagttccatcttcggtggttgctaatgaatatcattcccatcctccgcattcgcataatcattcgacttttgaattgggtcctggccccaagatccctcgctccgaactacgggattcagataagagatcgccacttatatcgatgtacagaatgcaggattcacagcattcggatcatcctggtggtggttcggatgcaaagggtgatcctgccaagggggagagggattcgcaaaagggtttcgagagtaggggtgatgatggtattagtactaacagcaataaagaagtgaaatttgatggtgattcgaagatggagaaggagggttttggttcgggaaatgttagtcatttaaattggaaagaatccaaggagtatcatcgagggaaacgttattcggaaaccccaggcgggaatgtagacccctgggttatgtcacggcctaatttgcatggtacaggtgaggtgggaaaggagagtctggcccctgcggatgacagggagtacctggaaacgcatgaggctgttggggaaaataaggttgatttgaaggtcgaggataagttcaaggacaaggacaggaagaggaaagatgcaaagcatagggattggggggaaagggataaggagaggagtgatcgccggaataacaacttgcaagtaggtaatagcagtggtgagggtaaagatttgagtagggaagaaagagaagcggagaggtgggagagagagaggaaggatgtctcaaaagacaaagaaaggccaaaagagagggaaaaggatcatagtaagagagaagcatggaatggagtggagaaagatggtctgcatagtgacaaagaagtggtcgatggatctgtgagaatgtctgagcaggaaagtccagcttcggagcaaaagaaacaaaaagaatttgatggctggaagaatgttgatagggaagctagggatagaagaaaagaaagggatgctgatgcagaaggtgatagacctgaaaagcgcagtagggtttatgacagagaatcagatgatggttgtgcagatgttgaagggggtacagacagggaaagagaagttttcaatcatggagttcatcgtaagaggatgcttcgcccgaggggaagtcctcaaatggcaaatcgtaggtctcgtgctcaggatgtcgaagggtctcaaggtaaacctgaagtatccactgttgtttataaagtcggtgaatgcatgcaagaactgataaaattgtggaaggaatatgaatcatctcaagctgataaaaatggtgaaagctcttctaatggtcctactttagaaatccgaataccagctgagcatgttactgctacgaatcgccaagtcagaggcggccaattatgggggacagatatatacactgatgactcagatcttgttgctgttctcatgcatacgggctattgtcgcccaacggcttctcctcctccacctgctattcaggagctacgtgctaccatccgggtgctacctccacaagattgctacatttctacactgagaaacaatgtccgatcccgtgcttggggggctgcaattggttgtagctaccgtgtcgaacggtgctgcattgtgaagaaaggaggcgggaccattgatcttgaaccttgtctaacacatacatcaactgtggagcctactcttgctccagtggctgttgagcgtacaatgactacaagggcagctgcttcgaatgcgttgcggcaacaaagatttgtacgagaagtcacaatacagtacaacttatgtaatgaaccttggattaaatacagcataagcattgttgctgacaaaggcctaaagaagcccctttatacatctgcacgcttgaagaagggagaagttttgtatttagaaacacattcccgcaggtatgaactgtgttttattggagagaagatggtcaaagctacaacagcattgcatggacatgaaacagagacagagaaatctcagactcatagcttgcattcaacaaatggtgaacgaaattcaactgatggtgataacattatgatcgatgtattccgctggtctcgttgtaagagggcccttccccaaaaagtcatgcgttcactgggaatcccactgcccctcgaacatttagaggtcttggaggagaatctcgactgggaggatgtgcagtggtcccaaactggtgtttgtatagctggaaaggaatatgcgcttgctcgagttcatttcctatctccaaattag

SEQ ID NO: 14 - protein, 805 aa, Vitis vinifera
MSGVPKRPHDEVGGGSGGAAAAAAAAGHSSGASKYPHEDSGNAFAGKLNPSSSSAPVPSSVVANEYHSHPPHSHNHSTFELGPGPKIPRSELRDSDKRSPLISMYRMQDSQHSDHPGGGSDAKGDPAKGERDSQKGFESRGDDGISTNSNKEVKFDGDSKMEKEGFGSGNVSHLNWKESKEYHRGKRYSETPGGNVDPWVMSRPNLHGTGEVGKESLAPADDREYLETHEAVGENKVDLKVEDKFKDKDRKRKDAKHRDWGERDKERSDRRNNNLQVGNSSGEGKDLSREEREAERWERERKDVSKDKERPKEREKDHSKREAWNGVEKDGLHSDKEVVDGSVRMSEQESPASEQKKQKEFDGWKNVDREARDRRKERDADAEGDRPEKRSRVYDRESDDGCADVEGGTDREREVFNHGVHRKRMLRPRGSPQMANRRSRAQDVEGSQGKPEVSTVVYKVGECMQELIKLWKEYESSQADKNGESSSNGPTLEIRIPAEHVTATNRQVRGGQLWGTDIYTDDSDLVAVLMHTGYCRPTASPPPPAIQELRATIRVLPPQDCYISTLRNNVRSRAWGAAIGCSYRVERCCIVKKGGGTIDLEPCLTHTSTVEPTLAPVAVERTMTTRAAASNALRQQRFVREVTIQYNLCNEPWIKYSISIVADKGLKKPLYTSARLKKGEVLYLETHSRRYELCFIGEKMVKATTALHGHETETEKSQTHSLHSTNGERNSTDGDNIMIDVFRWSRCKRALPQKVMRSLGIPLPLEHLEVLEENLDWEDVQWSQTGVCIAGKEYALARVHFLSPN

SEQ ID NO: 15 - DNA, 2502 bp, Ricinus communis
  CDS (1)..(2502)
atgagtagtgctcctaagagatctcatgaagagggtggtcactcctcttcttctaaatacccacacgaagaacctgcctcctatcctaagcttacatctagcgaataccatccctcctatgacatcactcccgatgctcgaattcctaaaattcctcgcactgagtcccgtgatgtcgatagaagatcacctctgcattcagtctatcgaatgccatcttccgccagtgatttgcacatggatacacattctcttgctcctgaaagcaggctggaatcaagggactccaaggaaaatagagaccacagggttgaaagccgagatcctaggactgaagcaagagatttgcacagcgagcctaagagggattcccaaaatttcaaaactgaaaaagatttaaggtttgagggtagagttgatgatagtaaggaaattaaatatgacaaggatgcttataatgatcccaagaatgactccaagatggaaaaggatgtttttggtgtgacagctagtcagttgaattggaaagaatcaaaggaataccatagaggaaagaggtactctgagtcccctggtggacatgtagatccttggcatatgtcacgtggtaactcccaggttgcaattgaaattggaaaagaagcctcgacaactgaagagagggattatgcagaaacacatgaggctgttggcgagaacaaagttgatttaaaaggcgaggatagatttaaagataaggataggaaaaggaaggatgtaaaacaccgggaatggggggacagagacagggaaagaagtgatcgtaggagtaacattccaggaggaaatagcagtggtgagggcaaagaatcagtgagggaagatagagaagcagagaggtgggagagggatagggagaggaaggatctttcaaaggacagggaaaggctaaaggagaaagaaaaggatcataccaagagagaatcatggaatggtgcagagaaagaaattttgaacaatgagaaagaatcagtcgatggatctgtgagagcgacagaacaggaaaatccatcttcagagcagaaaaaacagaaagattttgatggatggaaaaatgtcgatagggaagttagagacaggaggaaggaaagagaccttgacatggaaggagatagacctgacaagcggacccgagtatatgagaaagaatcagatgatggatgtgcagatggtgaagggaccacagaaagggacagggaactttttaactatggtgttcagcagcgcaagcggatgcttcgacctaggggcagcccacaaatggcaaatcgtgagccccgttttaggtctcgtactcaggaaaatgaaggagcttttggtgtttcaggaaaacctgaggtagcctctgttgtttataaagttggtgaatgcatgcaagatttgataaagttgtggaaggagtatgaatcatctcagactgaaaaaaatggtgaaagtacccttaatggtcccactcttgaagttaggataccagcagagcatgtgaatgctactaatcgtcaagtaagaggtggccagctatgggggacagatatatacacatatgattctgatcttgttgctgttctcatgcatacaggttacttccgccccactgcttctcctccacccgccatccaagagttgcgtgctactatccgagtgttgcctccgcaagatagctacacttctatgctgagaaattatcttcgttctcgttcctggggagctggagctggaattggctgtagttaccgtgttgagcgctgctgcattgtgaagaaaggaggtggaactattgatcttgagccttgtcttacacacacgtcagcagttgaacctacccttgctcctgtggctgttgagcggacaatgactacaagggctgcagcttcgaatgcattgcggcagcagagatttgtgcgtgaagttacagtacagtacaacctttgcaatgaaccatggataaagtatagcattagtattgttgcggacaaggccattatctgtaggtatgagctctgttttactggagagaaaatggtgaaagctacacaattgattcacggacatgaagagacagtgaagtctcataatcaccacacacatttctcaaatggtgaaaaaagtgaatctgataacattctgattgatatttttcggtggtcgcgatgtaagaagccccttccgcagaaggtcatgcgttcagtagggatcccactatcctccgagtatgttgaggtattggaggaaaatcttgactgggaggatgtgcagtggtcacaaactggtgtttggatagctgggaaagaatacacactagcaaggtatcaccctgaaactcccaactcggtacgggaacaaattgaagctcactgcaagcgcaatttgagctccagcaatcccacccatctaagtaaactgaaagaactggcatctaactggcttggaaatgttgcccaatggccaaaaactgatgcataa

SEQ ID NO: 16 - protein, 833 aa, Ricinus communis
MSSAPKRSHEEGGHSSSSKYPHEEPASYPKLTSSEYHPSYDITPDARIPKIPRTESRDVDRRSPLHSVYRMPSSASDLHMDTHSLAPESRLESRDSKENRDHRVESRDPRTEARDLHSEPKRDSQNFKTEKDLRFEGRVDDSKEIKYDKDAYNDPKNDSKMEKDVFGVTASQLNWKESKEYHRGKRYSESPGGHVDPWHMSRGNSQVAIEIGKEASTTEERDYAETHEAVGENKVDLKGEDRFKDKDRKRKDVKHREWGDRDRERSDRRSNIPGGNSSGEGKESVREDREAERWERDRERKDLSKDRERLKEKEKDHTKRESWNGAEKEILNNEKESVDGSVRATEQENPSSEQKKQKDFDGWKNVDREVRDRRKERDLDMEGDRPDKRTRVYEKESDDGCADGEGTTERDRELFNYGVQQRKRMLRPRGSPQMANREPRFRSRTQENEGAFGVSGKPEVASVVYKVGECMQDLIKLWKEYESSQTEKNGESTLNGPTLEVRIPAEHVNATNRQVRGGQLWGTDIYTYDSDLVAVLMHTGYFRPTASPPPAIQELRATIRVLPPQDSYTSMLRNYLRSRSWGAGAGIGCSYRVERCCIVKKGGGTIDLEPCLTHTSAVEPTLAPVAVERTMTTRAAASNALRQQRFVREVTVQYNLCNEPWIKYSISIVADKAIICRYELCFTGEKMVKATQLIHGHEETVKSHNHHTHFSNGEKSESDNILIDIFRWSRCKKPLPQKVMRSVGIPLSSEYVEVLEENLDWEDVQWSQTGVWIAGKEYTLARYHPETPNSVREQIEAHCKRNLSSSNPTHLSKLKELASNWLGNVAQWPKTDA

SEQ ID NO: 17 - DNA, 2385 bp, Oryza sativa
  CDS (1)..(2385)
atgagtggtgcacccaagaggtcgcatgaggagggtagtcactccacaccggcaaaacggccgttggatgacagcagcttgtactcaagcccttctgggaaaattattcaaccaggcagcagtgatttccatggttcgtttgaacatgatgggagatttgccaaagttcaacgtattgagccccgggatgataagaggccctctctggcacataggatgcctattggcccctccaactttgtggaccactcaatctcatctgatggcagattagaatcaaagcaaaataaagatccatgggacactaaggtagatgttcgggaggcaaaggctgacactcgagatgtctacagtgatcccagggttgaatttccgagcaataaagttgagactgatgtaaagacggacaatagagcagatgacaatgacataagagccgacagacggatacatgctgactacaaaggtgatgccaaactggacaaagatggtcatcctacagcaatttcaaacatagcctggaaagataacaaagaacataggggtaaaaggaatattgagcagccatctgataatgcagattggcgttttccccgccctggtttgcaaggaacagatgaatcttccaaaggtccagttcctgcagatgagcggtccaaggatgctcatgaatctactggtgagaataaaactgaacctaaaactgaagataagtttagagataaggacaggaaaaagaaggatgaaaagcatagggacttcggcacaagagacaatgatagaaatgatcgccgaattggtattcagcttggaggcaatagtgttgaacgaagagagaatcagagggaagatagggatgctgaaaagtgggatagggaaagaaaagattcccagaaggacaaggaaggcaatgatagagagaaggattctgcaaaggagtcatcagtagcaactgaaaaggagaatgcaatactggaaaaaactgcatctgatggagctgttaaaagtgccgagcatgagaataaaacagtagagcagaagacacttaaagatgatgcatggaaatcacatgatagggatcccaaggacaagaaaagagagaaggatatggatgcaggagaaaggcacgaccaaaggagtaaatataatgacaaggaatcagatgatacttgccctgaaggagatatagagaaggataaggaagcccttggaagtgtccaacgcaagagaatggcgcgatcaaggggtggtagtcaagcatcccaacgagaacctcgatttaggtctaggatgcgtgatggtgaaggatctcaaggtaaatctgaggcatcagccattgtctataaagctggtgagtgcatgcaagagcttctgaaatcatggaaagagtttgaagcaaccccagaagctaaaagtgctgaaagtgtgcaaaatggccccactcttgagatccgcatacccgcagagtttgttacgtccactaaccgtcaagtaaaaggtgctcaactttggggaacggatatttatacaaatgattcagatcttgtcgctgtgcttatgcatactggttactgctcccctacatcatcacctccaccatctgcaatccaagagctacgagcaactgttcgagttctaccgccacaagacagctatacttcaactttaaggaacaatgtccgctcacgtgcttggggtgctggtattggttgtagctttcgcatagaacgctgctgcattgttaagaaaggtggtggtactattgatcttgagcctcgcctaagccatacatcagctgtggagcctacacttgctccggttgcggttgagcgcacaatgacaacaagagcagcagcttctaatgcgttacgtcaacaaagatttgttcgggaagtcacaatacagtacaatctctgcaacgagccatggttgaaatacagcataagcattgtggcagacaagggattgaaaaagtcattatatacttctgcgaggctgaaaaaaggcgaagtcatatacttggaaacacattataataggtatgagctgtgcttcagtggagaaaaggctcgtcttgttggatcaagctccaatgcggcagacgcagaaactgagaaacaccagaatagtagccaccatcactcgcaaaatggggacagggcctcttcagaacatgaactgcgggatttgttccgatggtcccgctgtaagaaggcgatgcctgagagctctatgcgctccatcggtatcccgctgccagctgatcaacttgaggtgctgcaggataatttggaatgggaggatgtgcagtggtcgcagactggtgtttgggttgctggaaaggaatatcctctcgcccgagtgcatttcctatcatcaaactag

SEQ ID NO: 18 - protein, 794 aa, Oryza sativa
MSGAPKRSHEEGSHSTPAKRPLDDSSLYSSPSGKIIQPGSSDFHGSFEHDGRFAKVQRIEPRDDKRPSLAHRMPIGPSNFVDHSISSDGRLESKQNKDPWDTKVDVREAKADTRDVYSDPRVEFPSNKVETDVKTDNRADDNDIRADRRIHADYKGDAKLDKDGHPTAISNIAWKDNKEHRGKRNIEQPSDNADWRFPRPGLQGTDESSKGPVPADERSKDAHESTGENKTEPKTEDKFRDKDRKKKDEKHRDFGTRDNDRNDRRIGIQLGGNSVERRENQREDRDAEKWDRERKDSQKDKEGNDREKDSAKESSVATEKENAILEKTASDGAVKSAEHENKTVEQKTLKDDAWKSHDRDPKDKKREKDMDAGERHDQRSKYNDKESDDTCPEGDIEKDKEALGSVQRKRMARSRGGSQASQREPRFRSRMRDGEGSQGKSEASAIVYKAGECMQELLKSWKEFEATPEAKSAESVQNGPTLEIRIPAEFVTSTNRQVKGAQLWGTDIYTNDSDLVAVLMHTGYCSPTSSPPPSAIQELRATVRVLPPQDSYTSTLRNNVRSRAWGAGIGCSFRIERCCIVKKGGGTIDLEPRLSHTSAVEPTLAPVAVERTMTTRAAASNALRQQRFVREVTIQYNLCNEPWLKYSISIVADKGLKKSLYTSARLKKGEVIYLETHYNRYELCFSGEKARLVGSSSNAADAETEKHQNSSHHHSQNGDRASSEHELRDLFRWSRCKKAMPESSMRSIGIPLPADQLEVLQDNLEWEDVQWSQTGVWVAGKEYPLARVHFLSSN

SEQ ID NO: 19 - DNA, 2385 bp, Oryza sativa
  CDS (1)..(2385)
atgagtggtgcacccaagaggtcgcatgaggagggtagtcactccacaccggcaaaacggccgttggatgacagcagcttgtactcaagcccttctgggaaaattattcaaccaggcagcagtgatttccatggttcgtttgaacatgatgggagatttgccaaagttcaacgtattgagccccgggatgataagaggccctctctggcacataggatgcctattggcccctccaactttgtggaccactcaatctcatctgatggcagattagaatcaaagcaaaataaagatccatgggacactaaggtagatgttcgggaggcaaaggctgacactcgagatgtctacagtgatcccagggttgaatttccgagcaataaagttgagactgatgtaaagacggacaatagagcagatgacaatgacataagagccgacagacggatacatgctgactacaaaggtgatgccaaactggacaaagatggtcatcctacagcaatttcaaacatagcctggaaagataacaaagaacataggggtaaaaggaatattgagcagccatctgataatgcagattggcgttttccccgccctggtttgcaaggaacagatgaatcttccaaaggtccagttcctgcagatgagcggtccaaggatgctcatgaatctactggtgagaataaaactgaacctaaaactgaagataagtttagagataaggacaggaaaaagaaggatgaaaagcatagggacttcggcacaagagacaatgatagaaatgatcgccgaattggtattcagcttggaggcaatagtgttgaacgaagagagaatcagagggaagatagggatgctgaaaagtgggatagggaaagaaaagattcccagaaggacaaggaaggcaatgatagagagaaggattctgcaaaggagtcatcagtagcaactgaaaaggagaatgcagtactggaaaaaactgcatctgatggagctgttaaaagtgccgagcatgagaataaaacagtagagcagaagacacttaaagatggtgcatggaaatcacatgatagggatcccaaggacaagaaaagagagaaggatatggatgcaggagaaaggcacgaccaaaggagtaaatataatgacaaggaatcagatgatacttgccctgaaggagatatagagaaggataaggaagcccttggaagtgtccaacgcaagagaatggcgcgatcaaggggtggtagtcaagcatcccaacgagaacctcgatttaggtctaggatgcgtgatggtgaaggatctcaaggtaaatctgaggcatcagccattgtctataaagctggtgagtgcatgcaagagcttctgaaatcatggaaagagtttgaagcaaccccagaagctaaaagtgctgaaagtgtgcaaaatggccccactcttgagatccgcatacccgcagagtttgttacgtccactaaccgtcaagtaaaaggtgctcaactttggggaacggatatttatacaaatgattcagatcttgtcgctgtgcttatgcatactggttactgctcccctacatcatcacctccaccatctgcaatccaagagctacgagcaactgttcgagttctaccgccacaagacagctatacttcaactttaaggaacaatgtccgctcacgtgcttggggtgctggtattggttgtagctttcgcatagaacgctgctgcattgttaagaaaggtggtggtactattgatcttgagcctcgcctaagccatacatcagctgtggagcctacacttgctccggttgcggttgagcgcacaatgacaacaagagcagcagcttctaatgcgttacgtcaacaaagatttgttcgggaagtcacaatacagtacaatctctgcaacgagccatggttgaaatacagcataagcattgaggcagacaagggattgaaaaagtcattatatacttctgcgaggctgaaaaaaggcgaagtcatatacttggaaacacattataataggtatgagctgtgcttcagtggagaaaaggctcgtcttgttggatcaagctccaatgcggcagacgcagaaactgagaaacaccagaatagtagccaccatcactcgcaaaatggggacagggcctcttcagaacatgaactgcgggatttgttccgatggtcccgctgtaagaaggcgatgcctgagagctctatgcgctccatcggtatcccgctgccagctgatcaacttgaggtgctgcaggataatttggaatgggaggatgtgcagtggtcgcagactggtgtttgggttgctggaaaggaatatcctctcgcccgagtgcatttcctatcatcaaactag

SEQ ID NO: 20 - protein, 794 aa, Oryza sativa
MSGAPKRSHEEGSHSTPAKRPLDDSSLYSSPSGKIIQPGSSDFHGSFEHDGRFAKVQRIEPRDDKRPSLAHRMPIGPSNFVDHSISSDGRLESKQNKDPWDTKVDVREAKADTRDVYSDPRVEFPSNKVETDVKTDNRADDNDIRADRRIHADYKGDAKLDKDGHPTAISNIAWKDNKEHRGKRNIEQPSDNADWRFPRPGLQGTDESSKGPVPADERSKDAHESTGENKTEPKTEDKFRDKDRKKKDEKHRDFGTRDNDRNDRRIGIQLGGNSVERRENQREDRDAEKWDRERKDSQKDKEGNDREKDSAKESSVATEKENAVLEKTASDGAVKSAEHENKTVEQKTLKDGAWKSHDRDPKDKKREKDMDAGERHDQRSKYNDKESDDTCPEGDIEKDKEALGSVQRKRMARSRGGSQASQREPRFRSRMRDGEGSQGKSEASAIVYKAGECMQELLKSWKEFEATPEAKSAESVQNGPTLEIRIPAEFVTSTNRQVKGAQLWGTDIYTNDSDLVAVLMHTGYCSPTSSPPPSAIQELRATVRVLPPQDSYTSTLRNNVRSRAWGAGIGCSFRIERCCIVKKGGGTIDLEPRLSHTSAVEPTLAPVAVERTMTTRAAASNALRQQRFVREVTIQYNLCNEPWLKYSISIEADKGLKKSLYTSARLKKGEVIYLETHYNRYELCFSGEKARLVGSSSNAADAETEKHQNSSHHHSQNGDRASSEHELRDLFRWSRCKKAMPESSMRSIGIPLPADQLEVLQDNLEWEDVQWSQTGVWVAGKEYPLARVHFLSSN

SEQ ID NO: 21 - DNA, 2370 bp, Brachypodium distachyon
  CDS (1)..(2370)
atgagtggtgctccgaaaaggttgcctgaggagggtagccactcgacacctgcgaaacggcctttggatgagagcagcttgtattcgagcccttctgggaaactcattcaaccaggcagcactgatttccatggttctattgagcatgatggaagatctgccaaaatacaacgtgttgaacgatctctgccgcatcggattcatgtttcctcctctaactttgtagaccatccaacctcatctgacagcagattagaagcaaaacaaaacaaagatggaagggaaaccaaggttgaggatcgggaggcaaaagctgatgcgcgtgatgttcatagtgataccaggattgagtttcaaggcaataaagttgagactgatgtaaagacagacagtagagcagatgacaatgaaataagagctgaccgaagggttcataccgaatacaaaggtgatgccaaattggacaaggacggtcatcctgctggaacttcacacttggcctggaaagataataaagaccatcggggtaaaagatatgctgaacagccagatgataatgcaggttggcgttttctccgtcctgctttgcaaggcacagatgaaactcccaaggttccaactcctgtggaagaatggaactccaaggatgcacatgaatcaacaggtgagagcaaaattgaacctagaagtgaagataagttcagagacaaagacagaagaaagaaggatgaaaaacatagggattttggtgcaagagacggtgatagaaatgatcgcagaattggtattcagcttgcaggcagtagtgttgaacgaagagaaattcaaagggatgaccgggatgctgaaaaatgggacagggaaagaaaagattcccagaaggacaaggaaggcaacgatcgggagaaggattctgccaagaaggattcatttttagctgttgacaaggagaatgcaatactggaaaaggcagcatcagatggagctgttaaaactgctgaacatgagaatacagctactgaattgaagacacttaaagatgacaaatctcatgacagggatcctaaggacaagaaaagagagaaggatgtcgatacaggagacaggaatgaccaaagaagtaagtataatgacaaggaatctgatgatactggtcctgaaggagatacagacaaagataaggatacttttggaagtattcagcgcaggaggatggcacgtccaagaggtggtggtggtcaggcatctcaacgggaacctcgatttcggtccaaaatgcgtgatggtgaagggtctcaaggtaagtctgaggtttctgctattgtatataaagctggtgaatgcatgcaagaacttctgaaatcatggaaagagtttgaagcaaccccagatgctaaaaatgccgagaatcaacaagatggtcccactcttgaaatccgtatacctgcggagtttgttacctctaccaatcggcaagttaaaggtgctcaactttggggaacagatgtttatacaaatgattcagaccttgtggctgtactaatgcatactggttactgctcacctacatcatcacctccaccatctgctatccaagaactgcgtgcaactgttcgcgttctaccaccacaagacagctatacttcaaccctgaggaacaatgtccgctcacgtgcttggggtgctggtattggttgcagctttcgcatagaacgctgctgcattgttaagaaaggtggtggtaccattgatcttgagcctcggcttagccatacatcagctgtggagcccacacttgccccggtagcagtggagcgcacaatgacaacaagagcagcagcttctaatgcattacgtcagcaaagatttgtccgggaagtcacaatacagtacaatctctgcaatgaaccatggttaaaatatagtataagcattgtggcggataaaggattgaaaaagtcgctttatacttctgcaaggctgaaaaaaggcgaagtcatatacttggaaacacatttcaataggtatgagctgtgcttcagtggagaaaagccccgctctgttggatcaaactccagcgcatcagatttagaaccggaaaaacatcacaacagcagccaccaccattcacaaaatggggacaggggcactgcagaacatgaactccgggacatgttccggtggtcgcgatgtaagaaagctatgcctgagaccgccatgcgctctattggtatcccactgccagctgaacaactcgaggtgctgcaggacaatctagaatgggaggacgtgcagtggtcgcagacgggcgtctgggtttccgggaaggagtatcccctcgcccgcgtgcatttcctctcgtcgaactag

SEQ ID NO: 22 - protein, 789 aa, Brachypodium distachyon
MSGAPKRLPEEGSHSTPAKRPLDESSLYSSPSGKLIQPGSTDFHGSIEHDGRSAKIQRVERSLPHRIHVSSSNFVDHPTSSDSRLEAKQNKDGRETKVEDREAKADARDVHSDTRIEFQGNKVETDVKTDSRADDNEIRADRRVHTEYKGDAKLDKDGHPAGTSHLAWKDNKDHRGKRYAEQPDDNAGWRFLRPALQGTDETPKVPTPVEEWNSKDAHESTGESKIEPRSEDKFRDKDRRKKDEKHRDFGARDGDRNDRRIGIQLAGSSVERREIQRDDRDAEKWDRERKDSQKDKEGNDREKDSAKKDSFLAVDKENAILEKAASDGAVKTAEHENTATELKTLKDDKSHDRDPKDKKREKDVDTGDRNDQRSKYNDKESDDTGPEGDTDKDKDTFGSIQRRRMARPRGGGGQASQREPRFRSKMRDGEGSQGKSEVSAIVYKAGECMQELLKSWKEFEATPDAKNAENQQDGPTLEIRIPAEFVTSTNRQVKGAQLWGTDVYTNDSDLVAVLMHTGYCSPTSSPPPSAIQELRATVRVLPPQDSYTSTLRNNVRSRAWGAGIGCSFRIERCCIVKKGGGTIDLEPRLSHTSAVEPTLAPVAVERTMTTRAAASNALRQQRFVREVTIQYNLCNEPWLKYSISIVADKGLKKSLYTSARLKKGEVIYLETHFNRYELCFSGEKPRSVGSNSSASDLEPEKHHNSSHHHSQNGDRGTAEHELRDMFRWSRCKKAMPETAMRSIGIPLPAEQLEVLQDNLEWEDVQWSQTGVWVSGKEYPLARVHFLSSN

SEQ ID NO: 23 - DNA, 2382 bp, Sorghum bicolor
  CDS (1)..(2382)
atgagcagtgccccaaagaggttgcacgaggagggtagccactccacaccgacaaaacgtcctttggatgacagcagcttgtattcgagtcctgggaaagttattcagtccagtggcagtgatttccatggttcttttgaacatgatggtagatttgccaaaattcaacgtgtggagcctcgtgatgataagaggccatccgtaccatatcggatgcctgttggctccaccaactttgctgaccaccccgtctcctctgacagcagattagaatcaaagcaaaataaagatgcacgggacaataaggcagatgaccgcgagacaaaagctgatgctagggacgtccatagtgattcaaggattgaatttcaggccaataaaattgagagtgatgtaaaggtagacaatagagcagatgaaagcgaaataagggctgacaggaggggccatcctgattacagaagtgacatcaaatttgacaaggataatcattctactgttccagcaaacataaactggaaggacaacaaggagcataggagtaaaagatattttgaacagccagctgatactgtggattggcgtttgccccgtcctagtttacaaagtattgatgaagctcccaaaggtctgatttctgtggaagagcgtaactccaaggatgcaaatgaatctgctggtgataacaaagctgaaccaaaaagtgaagataggttcagagacaaggacaggaaaaagaaggacgagaagcatagggactttggtgcaagagaaggtgatagaaatgatcgtcggactggtgtacagcttggtagtagtggtgttgagcgaagagaaatgcaaagggaagatagggatgctgagaaatgggacagggaaagaaaagattccgtgagagataaggaaggcaatgatagggagaaagattctgctaggaaggattcatctgtagtaattgaaaaggataacactatactagaaaaagcttcatctgatggagccattaagagtgctgagcatgagaatacaacagaatccaaggtacctaaggatgatgtatggaaagctcacgatagggatcctaaggacaagaaaagagagaaggatggggatgcaggggaccggatcgagcaaagaagcaaatataatgataaggaatcagatgacaatggcactgaaggagatatggagaaagataaggaagtttttggaagtgtccaacgcaggaggatggtgcgaccgaggggaggtagtcaagcatctcagcgtgaacctagatttcggtccagaatgcgtgatggtgaagggtctcaaggtaagtctgaggtgtctgccattgtttataaagccggggagtgcatgcaggagcttctgaaatcatggaaagagtttgatgtaactcaggatgctacaaatgctgaaagtctacaacatggtcctactcttgaaattcgaatacctgcggagtttgttacttccactaatcgtcaggtaaaaggtgctcagctttggggaacagacgtttatacaaacgattcagatcttgtggctgtgctaatgcatactggttactgctcccctacatcctcccctccaccatctgccattcaagagcttcgtgcaactgttcgagttctaccaccacaagagagttatacttcaacactgaggaacaatgtgcgctcacgtgcttggggtgctgggattggttgtagctttcggattgaacgctgctgcattgtcaagaaaggtggtggaaccattgatcttgagccacgccttagccacacatcagctgtggagcctactctcgctccagttgcagttgagcgtacaatgacgacaagagctgcagcttctaatgcactgcgtcaacaaagatttgttcgtgaagtgactatacagtacaatctgtgcaatgagccatggttaaaatatagtataagcattgtggcagataagggattgaaaaagtctctgtatacttctgctagactgaagaaaggagaagtcatatatttagaaacacactttaataggtatgaactttgcttcaatggagagaagcctcgtcttattggatcaagctccaatgcatctgaatcagaaacggagaaacaccagagtggtagtcaccattctcagaatggtgacagatgctatgtggagcatgaactccgggatgtgttccgatggtcccgttgtaagaaggccatgcctgaaagtgccatgcgctccatcggtatcccactaccagcagaccaactagaggtattgcaagataacctagaatgggaggacgtgcagtggtcacagactggtgtgtgggtatctgggaaggagtatcccctcgcccgagtgcacttcctctcggcgaactag

SEQ ID NO: 24 - protein, 793 aa, Sorghum bicolor
MSSAPKRLHEEGSHSTPTKRPLDDSSLYSSPGKVIQSSGSDFHGSFEHDGRFAKIQRVEPRDDKRPSVPYRMPVGSTNFADHPVSSDSRLESKQNKDARDNKADDRETKADARDVHSDSRIEFQANKIESDVKVDNRADESEIRADRRGHPDYRSDIKFDKDNHSTVPANINWKDNKEHRSKRYFEQPADTVDWRLPRPSLQSIDEAPKGLISVEERNSKDANESAGDNKAEPKSEDRFRDKDRKKKDEKHRDFGAREGDRNDRRTGVQLGSSGVERREMQREDRDAEKWDRERKDSVRDKEGNDREKDSARKDSSVVIEKDNTILEKASSDGAIKSAEHENTTESKVPKDDVWKAHDRDPKDKKREKDGDAGDRIEQRSKYNDKESDDNGTEGDMEKDKEVFGSVQRRRMVRPRGGSQASQREPRFRSRMRDGEGSQGKSEVSAIVYKAGECMQELLKSWKEFDVTQDATNAESLQHGPTLEIRIPAEFVTSTNRQVKGAQLWGTDVYTNDSDLVAVLMHTGYCSPTSSPPPSAIQELRATVRVLPPQESYTSTLRNNVRSRAWGAGIGCSFRIERCCIVKKGGGTIDLEPRLSHTSAVEPTLAPVAVERTMTTRAAASNALRQQRFVREVTIQYNLCNEPWLKYSISIVADKGLKKSLYTSARLKKGEVIYLETHFNRYELCFNGEKPRLIGSSSNASESETEKHQSGSHHSQNGDRCYVEHELRDVFRWSRCKKAMPESAMRSIGIPLPADQLEVLQDNLEWEDVQWSQTGVWVSGKEYPLARVHFLSAN

SEQ ID NO: 25 - DNA, 2379 bp, Sorghum bicolor
  CDS (1)..(2379)
atgagtggtgctccaaagaggttgcacgaggagggtagccacaccacgccagcaaaacggcctttggatgacagcagcttgtattcgagtcctgggaaagttattcagtccagtggcagtgatttccatagttcttttgaacatgatggtagatttgcaaaaatccaacgtgtggagcctcgtgatgataagagaccatccctaacacatcggatgcctgttagctccaccaactttgctgaccaccccatctcgtctgacagcagattagaatcaaagcaaaataaagatgcaagggacactaaggcagatgatcatgagacaaaagctgatgctagggatgtctatagtgattcaaggattgaaattcaggctaataaaattcagggtgatgtaaaggtagacaagagagcagatcaaagcgaaataaaggctgacaggaggggccatcctgattacaaaggtgacatcaaatttgacaaggattgtcatcctactgttccaacaaacataggctggaaggacaacacagaacataggggtaaaagatattttgaacagccagctgataatgtggatggccatttgactttgccccgtcctagtttacaaggtactgatgaaactctcaaatttccaatttctgtggaagaacgtaaatccaaggatgcacatgaatctgctggtgacaacaaagctgaaccaagaagcgaagataaattcagagacaaggaccggaaaaggaaggatgagaagcatagggactttggtgcaagagaaggtgatagaaatgatcgtcggaccggtgtacagctcagtggtagtggtgttgagcgaagagaaatgcaaattagagatgctgacaaatgggacagggaaagaaaagattccctgagagacaaggaagacaatgatagggggaaggattctgctcggaaagattcatctgtagtaattgagaaggataacactacactggaaaaggcttcatctgatggagctgttaagagtgctgagcatgggaatacagcaacagaatccaaggcacctaagcatgatttatggaatgctcatgatagggatcctaaggacaagaaaagagagaaagatgtggaagcaggggacaggcatgaacaaagaagaatatataatgtcaaggaatcagatggtaatggcaccgaaggaggtatggagaaagataaagaagtttctggaagtttccaacgcaggagggtggtgcgaccaaggggaggtagtcaagcatctcagcgtgaacctcgatttcgatccagaatgcatgatggtgaagggtctcaaggtaagtctgaggtgtctgccattgtttacaaagctggggagtgcatgcaggagctgctgaaatcatggacagagttcagtgcaactcaggatgctacaaacgctgaaagtctacagaatggtcctgcccttgaaattcgaatacctgcggaatttgttacttccactaatcgtcaagtaaagggtgctcagctttggggaacagatatttatacaaatgattcagatcttgtggctgtgctaatgcatactggttactgctcccctacatcctcccctcccccatctgccatccaagagcttcgtgcaaccgttcgagttctaccaccacaagagagttatacttcaacattgaggaacaatgtgcgttcacgtgcttggggtgctgggattggttgtagctttcagatagaacgctgctgcattgttaagaaaggtggtggcaccattgacctcgagcctcgccttagccacacatcagctgtggaacctactcttgctccagttgtggttgagcgtacaatgacgacaagagctgcagcttccaatgctttgcgtcaacaaagatttgtccgtgaagtgactatacagtataatctctgcaatgagccatggttaaaatatagtataagcattgtggcagacaagggattgaaaaagtctctttatacttctgctagactgaagaaaggagaagtcatatatttagagacacacttcgataggtataagcctcttttacacaggtacgagctgtgcttcagtggagagaagcctcgtattgttgaagcagaagcggagaaacaccagagcggcagtcaccactcacaaaatggtgacagacgcgagcatgaattacgggatgtgttccgatggtcccgttgtaagaaggccatgcctgagagtgccatgcgctccatcggtatcccgctaccagcagaccagcttgaggtgttgcaggataacctagaatgggaggacgtgcagtggtcgcagaccagcgtctgggtggctgggaaggagcatcccctcgctcgagtgcacttcctctcggagaactag

SEQ ID NO: 26 - protein, 792 aa, Sorghum bicolor
MSGAPKRLHEEGSHTTPAKRPLDDSSLYSSPGKVIQSSGSDFHSSFEHDGRFAKIQRVEPRDDKRPSLTHRMPVSSTNFADHPISSDSRLESKQNKDARDTKADDHETKADARDVYSDSRIEIQANKIQGDVKVDKRADQSEIKADRRGHPDYKGDIKFDKDCHPTVPTNIGWKDNTEHRGKRYFEQPADNVDGHLTLPRPSLQGTDETLKFPISVEERKSKDAHESAGDNKAEPRSEDKFRDKDRKRKDEKHRDFGAREGDRNDRRTGVQLSGSGVERREMQIRDADKWDRERKDSLRDKEDNDRGKDSARKDSSVVIEKDNTTLEKASSDGAVKSAEHGNTATESKAPKHDLWNAHDRDPKDKKREKDVEAGDRHEQRRIYNVKESDGNGTEGGMEKDKEVSGSFQRRRVVRPRGGSQASQREPRFRSRMHDGEGSQGKSEVSAIVYKAGECMQELLKSWTEFSATQDATNAESLQNGPALEIRIPAEFVTSTNRQVKGAQLWGTDIYTNDSDLVAVLMHTGYCSPTSSPPPSAIQELRATVRVLPPQESYTSTLRNNVRSRAWGAGIGCSFQIERCCIVKKGGGTIDLEPRLSHTSAVEPTLAPVVVERTMTTRAAASNALRQQRFVREVTIQYNLCNEPWLKYSISIVADKGLKKSLYTSARLKKGEVIYLETHFDRYKPLLHRYELCFSGEKPRIVEAEAEKHQSGSHHSQNGDRREHELRDVFRWSRCKKAMPESAMRSIGIPLPADQLEVLQDNLEWEDVQWSQTSVWVAGKEHPLARVHFLSEN

SEQ ID NO: 27 - DNA, 2382 bp, Zea mays
  CDS (1)..(2382)
atgagtggtgctccaaagaggttgctcgaggaaggtagtcactccacaccaacaaaacgccctttggatgacagcagcttgtattcgagtcctgggaaatttattcagtccggtggcagtgatttccatggttcttctgaacatgatggtagatttgcgaaatttcaacgtgtggagtctcgtgatgataagaggccatctgtacatcggatgcctgttggctccactaactttgctgttcaccccatctcgtctgacagcagattagagtcaaagcaaaataaagatgcacgggacagtaaggcagatgaccgcgaaacaaaagtcgatgccagggacgttcatagtgattcaaggattgaatttcaggctaataaaattgagagtgatgtaaaggtagacaatagagcagatgaaagtgaaataagggctgacaggaggggccatcctgattacagaactgacataaaatttggtaaggatagtcattctactgttccagcaaacataaactggaaggacaacaaggagcacaggggtaaaagacattttgaaccgcccgctgatactgtggattggcgtttgccccgtcctagtttacaaagtatcgatgaagctcccaaaggtccaatttctgtggaaggacgtaattccaaggacacaaatgaatctgctggtgattacaaagctgaaccaaaaaacgaagataggttcagagacaaggacaggaaaaagaaggacgagaagcatagggacttcggtgcaagagaaggcgatagaaatgatcgtcggaccggtgtaccacttggcagtagtggtgttgagcgaagagaaatgcaaagggaagatagggatgctgagaaatgggacagggaaagaaaagattccctgcgagacaaggaaggcaatgatagggagaaggattctgctaggaaagattcatctgtagtaattgcaaaggataaccctatactagaaaaagcttcatctgatggagctgttaagagtgctgagcatgagaatacgacaacagaatccaaggcacctaaggatgatgtatggaaagctcacgatagggatcctaaggacaagaaaagagagaaggatgtggatgcaggagactggcttgagcaacgaaacaaatataatgataaggaattagatgacaatgccattgaaggagatatggagaaagataaggatgtttttggaagtgtccaacgaaggaggatggtgcgaccaaggggaggtagtcaagtatctcagcgtgaacctcgattccggtccagaatgcgtgatggtgaagggtctcaaggtaagtctgaggtgtctgccattgtttataaagctggggagtgcatgcaggagcttctgaaatcatggaaagagtttgatgtaactcaggatgctacaattgctgaaagcctacaacatggtcctactcttgaaatccgaatacctgcagaatttgttacttccactaaccgtcaggtaaaaggtgctcagctctggggaacagatatttatacaaatgattcagatcttgtggctgtgctaatgcatactggttactgctcccctacatcctcccctccaccatccgccattcaagagcttcgtgcaactgttcgagttctaccaccacaagagagttatacttcaacactgaggaacaatgtgcgttcacgtgcttggggtgctgggattggttgtagctttcggattgaacgttgctgcattttcaagaaaggtggtggcaccattggtcttgagccacgccttagccacgtgtcagctgtggagcctactctcgccccagttgcagttgagcgtacaatgacgacaagagctgcagcttctaatgcattgcggcaacaaagatttgtccgtgaagtgactatacagtacaatctgtgcaatgagccatggttgaaatatagtataaacattgtggcagataagggattgaaaaagtctctttatacttctgctagactgaagaaaggagaagtcatatatttagaaacacacattaataggtatgagctttgcttcagtggagacaagccttgcattattggatcaagctccaatgcatctgaatcagaaacggagaaacaccagagcgggagtcaccattctcagaatggtgacagaggctgtgtggagcatgaactccgggatgtgttccggtggtcccgctgtaagaaggccatgcctgaaagtgccatgcgctccatcggtatcccactaccagcagaccagttagaggtattgcaggataacctcgaatgggaggatgtgcagtggtcacagaccggtgtgtgggtatctgggaaggagtatcccctcgcccgagtgcacttcctctcggcgaactag

SEQ ID NO: 28 - protein, 793 aa, Zea mays
MSGAPKRLLEEGSHSTPTKRPLDDSSLYSSPGKFIQSGGSDFHGSSEHDGRFAKFQRVESRDDKRPSVHRMPVGSTNFAVHPISSDSRLESKQNKDARDSKADDRETKVDARDVHSDSRIEFQANKIESDVKVDNRADESEIRADRRGHPDYRTDIKFGKDSHSTVPANINWKDNKEHRGKRHFEPPADTVDWRLPRPSLQSIDEAPKGPISVEGRNSKDTNESAGDYKAEPKNEDRFRDKDRKKKDEKHRDFGAREGDRNDRRTGVPLGSSGVERREMQREDRDAEKWDRERKDSLRDKEGNDREKDSARKDSSVVIAKDNPILEKASSDGAVKSAEHENTTTESKAPKDDVWKAHDRDPKDKKREKDVDAGDWLEQRNKYNDKELDDNAIEGDMEKDKDVFGSVQRRRMVRPRGGSQVSQREPRFRSRMRDGEGSQGKSEVSAIVYKAGECMQELLKSWKEFDVTQDATIAESLQHGPTLEIRIPAEFVTSTNRQVKGAQLWGTDIYTNDSDLVAVLMHTGYCSPTSSPPPSAIQELRATVRVLPPQESYTSTLRNNVRSRAWGAGIGCSFRIERCCIFKKGGGTIGLEPRLSHVSAVEPTLAPVAVERTMTTRAAASNALRQQRFVREVTIQYNLCNEPWLKYSINIVADKGLKKSLYTSARLKKGEVIYLETHINRYELCFSGDKPCIIGSSSNASESETEKHQSGSHHSQNGDRGCVEHELRDVFRWSRCKKAMPESAMRSIGIPLPADQLEVLQDNLEWEDVQWSQTGVWVSGKEYPLARVHFLSAN

SEQ ID NO: 29 - DNA, 2427 bp, Glycine max
  CDS (1)..(2427)
atgagtggtgcacctaagagatctcatgaagagtctgttcattcatcttcaaagcactcaaatgaagattcgggtacttattccaagttggtttcattgccagtctcaaatgagtaccatatgccttatgatataagtcaggactcccgggtggcaaaagtgcctcgaactgaatttcgtgatgcagatagaagatcccctcttaatccagtgtatcggatgtcgtcacctttgaatgattctcgtgcagataatcctattggtcctgagaataggatagaatcaagggattcgaaggacagtagagatccccggtttgagaatcgtgatacaaagacagagaaggagttgtatggtgaagcaagaagggatcctccaaatgctaaaagtgaaaaggatatgcgcgtagaaggtagaggagatgacaacaaggatgtttggcatgatcgggatagtcataatgatccgaaaggtgacaccaagacagagaaagatggttataatgtggctagcagccacttgaattggaaagattcaaaagagtaccatagaggaaaaagatattctgatgctcctggtggaagtttggacacatggcatatgttacgtggaaatacacaaggctcggttgaggttgggaaggagagttccgcagcaggagagagagattatgttgaagctcatgaagctgttagtgagaacaaagttgatcctaaaggtgatgatagatccaaagagaaagatagaaagaggaaagatgtgaagcatagggaatggggagatagggaaaaagaaagaagtgatcgtagaaacagtccacaagttagcaatagtaccggtgactgcaaagaatctaccaaggaagatagagatgtagaaaggttggagagggagaaaaaagatcttccagaagagaaagaaaatataaaagagagggaaaaggatcagatgaagagggaatcatggaatggaatggagaaagaggtctcaattaacgagaaggaacctgttgatgcatcagctaaacttcctgaacaagaacctgtgttaccagagcagaagaaacaaaaagaagttgatagctggaaaaatgtagatagagaagctagagagaagagaaaagaaagggatgctgatttagaaggagataggtctgataagcatagcaaatgtcttgacaaggaatcaaacgatgggtgtgctgatggagaagggatgatggagaaggagagggaggtctataattatagcagtcagcaccgtaagaggatacaacgatctagagggagccctcaggtgcctaaccgggagcctcgtttcagatcccgtgcccaagataatgatgggtctcaaggtaaagtagaagtttcttctgttgtttataaagttggcgaaagcatgcaagaactgataaagttgtggaaggaatatgaatcatctcaatctcaaatggaaaaaaatggtgaaagctctaataatggtcccactctggaaattcgtataccatctgagcatatcacagctacaaaccgccaagtcagaggtggccagctttgggggaccgatgtgtacacatacgattcagatcttgttgctgttctcatgcatacaggttactgtcgcccaacagcgtctccaccccatgcagccatacaagaattgcgtgcaaccgttcgtgtactacctcctcaagattgctatatttctacactgagaaacaatgtccgttcccgtgcttggggtgcagcaattggttgtagttatagagtggagcggtgttgcattgtgaagaaaggaggtggaactattgatcttgaaccttgccttacacatacatcaactattgagcccacccttgctccagtgactgttgagcgaactatgactaccagggctgcagcttcgaatgcattgcggcaacaaagatttgttcgagaagtcacaatacagtacaatctctgcaatgagccttggataaagtatagtataagcactgttgctgacaagggtttaaaaaagccactttacacatctgcacgtttgaagaagggggaagttttgtatttggagacacatttgtccagatatgaactttgttttactggagagaagatgctcaaggttacaccagcagccccgttgcatgaccctgccacagaaaagtctcaaaatcaccacccacattctgcaaatggtgaaaaaaatgattgtgagaatgtcatgattgacgcattccggtggtctcgttgtaagaagcctctgccacagaaactgatgcgtacaattggcatccctttgcctcttgaacatatagaggtactggaggaaaatttggactgggaagatgtgcaatggtcgcaagctggtgtttggattgctggaaaggaatataccctggcacgggtgcatttcttgtcaatgaattaa

SEQ ID NO: 30 - protein, 808 aa, Glycine max
MSGAPKRSHEESVHSSSKHSNEDSGTYSKLVSLPVSNEYHMPYDISQDSRVAKVPRTEFRDADRRSPLNPVYRMSSPLNDSRADNPIGPENRIESRDSKDSRDPRFENRDTKTEKELYGEARRDPPNAKSEKDMRVEGRGDDNKDVWHDRDSHNDPKGDTKTEKDGYNVASSHLNWKDSKEYHRGKRYSDAPGGSLDTWHMLRGNTQGSVEVGKESSAAGERDYVEAHEAVSENKVDPKGDDRSKEKDRKRKDVKHREWGDREKERSDRRNSPQVSNSTGDCKESTKEDRDVERLEREKKDLPEEKENIKEREKDQMKRESWNGMEKEVSINEKEPVDASAKLPEQEPVLPEQKKQKEVDSWKNVDREAREKRKERDADLEGDRSDKHSKCLDKESNDGCADGEGMMEKEREVYNYSSQHRKRIQRSRGSPQVPNREPRFRSRAQDNDGSQGKVEVSSVVYKVGESMQELIKLWKEYESSQSQMEKNGESSNNGPTLEIRIPSEHITATNRQVRGGQLWGTDVYTYDSDLVAVLMHTGYCRPTASPPHAAIQELRATVRVLPPQDCYISTLRNNVRSRAWGAAIGCSYRVERCCIVKKGGGTIDLEPCLTHTSTIEPTLAPVTVERTMTTRAAASNALRQQRFVREVTIQYNLCNEPWIKYSISTVADKGLKKPLYTSARLKKGEVLYLETHLSRYELCFTGEKMLKVTPAAPLHDPATEKSQNHHPHSANGEKNDCENVMIDAFRWSRCKKPLPQKLMRTIGIPLPLEHIEVLEENLDWEDVQWSQAGVWIAGKEYTLARVHFLSMN

SEQ ID NO: 31 - DNA, 2406 bp, Glycine max
  CDS (1)..(2406)
atgagtggtgttcctaagagatctcatgaggattctgttcatcagtcttcaaagcatccacatcaagattcaggtacatattccaagttgatgccatcagtttcaaatgaccaccatattccttatgatatgagtcaggattcccgggtggcaaagacagtccgtactgaacctcgtgatgcagatagaagatctcatcttcatacagtgtatcggatgccattatcttcaaatgattctcatgcagatcatcccattggacctgagagcaggacagaatctagggattttaaggagagtagagaaccccggtttgagaatcgtgatacgaagacagagaagaaggaattgcatggtgaagccagaagggattctcagattgcaaagagtgagaaggatgtgcgagttgatggcagaggagatgataacaaggatattagatatgaatgggatggccataatgattcgaaaggtgacattaagacagacaaggatggctatggtatggtaagcagcagcagccacttgaattggaaagaatcaaaagagtataggggtaagagattttctgatgcccctggtgggagtttggattcctggcatacatcacgtggaaatacaccaaccgaagttggaaaggacagttcaatggcagaagaaagagactatttggaaacacatgaggctgttggggaaaacaaaattgattctaaaagtgaagatagatttaaagaaagaaaaagaaaggatgtcaagcatcgggattggggggatagagaaaaggagagaagtgatcgcagaagcactacgccagttaacaataatagtggtgacaacaaagaatctgccaaggaagatagagatgtagaaaaatgggagagggagaggaaagatcttccaaaagagaaagaaagttcaaaagagaaggaaaaggatcatagcaagagggaatccttgaacggaatggagaaagatggtttgaatgatgggaaggaactttgtgaagaaaaaaatactgagctagaaaatgtgttaccagaacaaaagaaacagaaagatgttgacagctggaaaaatgttgatggagaagttagagagaggagaaaagaaagggatgctgatttagaaggagatcggcctgataagcgcagtaaaattgacaagcaatcagaagatggaagtgctcacggggaaggaactggagagaaggagagggaagtccataattataatgttcaacatcgtaaaaggatccaccgatcaaggggaagccctcaggtggccaatcgtgaggctctgagagcaaagtccttctcaaattctgatatttcaggtaaagcagaagtctcttctgttgtttataaagttggtgaaagcatgcaagaactgataaagttgtggaaggaatatgaattatctcaatctcaagttgaaaaaaatagtgaaagctctaatggtggccccactcttgaaatccggataccagctgagaatgttacagctacaaaccgtcaagttagaggtggccagctatgggggactgatgtttacacttatgactcagatcttgttgctgttctcatgcatacaggttattgtcgcccaacagcttctccacctcacatggctgtacaagagttgcgcacaaccattcaagtgctacctccgcaagattcctatatttctactctgagaaacaatgtacgttcccgtgcttggggtgctgcaattggttgtagttataaagttgagcggtgctgcatcgtaaagaaaggaggtggaactattgatcttgaaccttgccttacacatacctcaactgttgagcctacccttgcaccagttgctactgagcggacaattactactagggctgcagcttcgaatgcattgcggcagcaaagatttgtacgcgaagttacaatacagtacaacctctgcaatgaaccatggatcaaatatagtataagcattgttgctgacaagggtctaaaaaagccactctatacatctgctcgtttaaagaagggagaagttctttatctggagacacactcctgcagatatgagctctgttttactggagaaaagatggcgaaggctataccagcaactcagatgcatgacctagatacagagaagtctcaaaatcaccatcaccatcccacaaatggtgacaaagctgattctgataatgttatggttgatgtatttcgatggtctcgatgtaagaatcctctaccccagaaactgatgcgcacgattggaatccctctgcctcttgaacatgtggaggtgctagaggaaaacctggactgggaagatgtacagtggtcgcaaactggcgtttggattgcaggaaaggaatatacccttgctcgggtgcatttcttgtcaatgaattag

SEQ ID NO: 32 - protein, 801 aa, Glycine max
MSGVPKRSHEDSVHQSSKHPHQDSGTYSKLMPSVSNDHHIPYDMSQDSRVAKTVRTEPRDADRRSHLHTVYRMPLSSNDSHADHPIGPESRTESRDFKESREPRFENRDTKTEKKELHGEARRDSQIAKSEKDVRVDGRGDDNKDIRYEWDGHNDSKGDIKTDKDGYGMVSSSSHLNWKESKEYRGKRFSDAPGGSLDSWHTSRGNTPTEVGKDSSMAEERDYLETHEAVGENKIDSKSEDRFKERKRKDVKHRDWGDREKERSDRRSTTPVNNNSGDNKESAKEDRDVEKWERERKDLPKEKESSKEKEKDHSKRESLNGMEKDGLNDGKELCEEKNTELENVLPEQKKQKDVDSWKNVDGEVRERRKERDADLEGDRPDKRSKIDKQSEDGSAHGEGTGEKEREVHNYNVQHRKRIHRSRGSPQVANREALRAKSFSNSDISGKAEVSSVVYKVGESMQELIKLWKEYELSQSQVEKNSESSNGGPTLEIRIPAENVTATNRQVRGGQLWGTDVYTYDSDLVAVLMHTGYCRPTASPPHMAVQELRTTIQVLPPQDSYISTLRNNVRSRAWGAAIGCSYKVERCCIVKKGGGTIDLEPCLTHTSTVEPTLAPVATERTITTRAAASNALRQQRFVREVTIQYNLCNEPWIKYSISIVADKGLKKPLYTSARLKKGEVLYLETHSCRYELCFTGEKMAKAIPATQMHDLDTEKSQNHHHHPTNGDKADSDNVMVDVFRWSRCKNPLPQKLMRTIGIPLPLEHVEVLEENLDWEDVQWSQTGVWIAGKEYTLARVHFLSMN

SEQ ID NO: 33 - DNA, 2430 bp, Glycine max
  CDS (1)..(2430)
atgagtggtgcacctaagagatctcatgaagagtctgttcattcatcttcaaagcacccgaatgaagatttgggtacatattccaagttggtttcatcgtcagtttcaaatgagtaccatatgcctcatgatataactcaggactcccgggtggcaaaagtgcctcgaactgaatttcatgatgcagatagaagatctcctcttaatcctgtgtatcggatgtcgtcaccgttgaatgattctcgtacagatcatcctattggccctgagaacaggattgaatcaagggattccaaggacaatagagatctccggtttgagaaccgcgatacaaagacagagaagaaggagttgcatggtgaagcaagaagggatcctccaagtgctaagagtgaaaaggatgtgcgtgttgaaggtagaggagatgacaacaaggatgtcaggcatgatcgggatagtcataatgatccgaaaggtgacaccaagacagagaaagatggttataatgtggttagcagccacttgaattggaaagattcaaaagagtaccatagaggaaaaagatattctgattcccctggtgggaattgggacacatggcatatgtcacgtggaaatacacaaggctcagttgaggttgggaaggagagttcagcagcaggagaaagagatcatgttgaagctcatgaagctgtttgtgagaacaaagttgatcctaaaggtgatgatagatctaaagagaaagatagaaagaggaaggatgtgaagcatagggaatggggagatagggaaaaagaaagaagtgatcgtagaaacagtccacaagtaacaaacagtaccggtgactgcaaagaatctgccaaggaagatagagatgtagaaaggttggagagggagaaaaaagatcttccaaaagagaaagaaaatttaacagagagggaaagggatcagatgaagagagaatcatggaatggaatggagaaagaggtttcaaataacgagaaggaatctgttgatgcatcagataaactaactgaacaagaaattgtgttaccagagcagaagaaacaaaaagaagttgatagctggaaaaatgtagatagagaagctagagagaggagaaaagaaagggatgctgatttagaaggggataggtctgataaacgtaccaagggccttgacaaggaatcaaacgatgggtgtgctgatgtagaaggggtgatggagaaggagagggaggtctataattatagcagtcagcaccgtaagaggatacaacgatctaggggaagccctcaggcgccgaaccgggagtcttttttcagatcccatccccaagacaaagacgggtctcaaggtaaagtagaagtttcttctgttgtttataaagttggcgaaagcatgcaagaactgataaagttgtggaaggaacatgaatcatctcaatctgaaatggagaaaaatggtgaaagctctaataatggtcccactctggaaattcggataccatctgagcatgtaacggctacaaaccgccaagtcagaggtggccagctttgggggaccgatgtgtacacatacgattcagatcttgttgctgttctcatgcataccggttactgtcgcccaacagcatctccacctcatgcagccatacaagaattgcgtgcaactgtccgtgtgctacctcctcaagattgctatatttctacactgagaaacaacatacgttcccgtgcttggggtgcagcaattggttgtagttatagagttgagcggtgttgcattgtgaagaaaggaggtgatactattgatcttgaaccttgccttacacatacatcaactattgaacccacccttgctccagtgactgttgagcggacaatgactaccagggctgcagcttcgaatgcattgcggcaacaaagatttgttcgagaagtcacaatacagtacaatctctgcaatgagccatggataaaatatagtataagcactgtcgcggacaagggtttaaaaaagccactctacacatctgctcgtttgaagaagggagaagttttgtatttggagacacatttgtccagatatgaactttgttttactggagagaagatggtcaaggttacaccagcaacccagttgcatgaccctgtcacagaaaagtctcaaaatcaccacccacattctacaaatggtgaaaaaaatgattgtgagaatgtcatgattgatgcattcaggtggtctcgttgtaagaagcctctgccacagaaactgatgcgtacaattggcatccctttgcctattgaacatatagagttactggaggaaaatttggactgggaagatgtgcaatggtcgcaaacaggtgtttggattgctggaaaggaatataccttggcacgagtgcatttcttgtcaatgaattaa

SEQ ID NO: 34 - protein, 809 aa, Glycine max
MSGAPKRSHEESVHSSSKHPNEDLGTYSKLVSSSVSNEYHMPHDITQDSRVAKVPRTEFHDADRRSPLNPVYRMSSPLNDSRTDHPIGPENRIESRDSKDNRDLRFENRDTKTEKKELHGEARRDPPSAKSEKDVRVEGRGDDNKDVRHDRDSHNDPKGDTKTEKDGYNVVSSHLNWKDSKEYHRGKRYSDSPGGNWDTWHMSRGNTQGSVEVGKESSAAGERDHVEAHEAVCENKVDPKGDDRSKEKDRKRKDVKHREWGDREKERSDRRNSPQVTNSTGDCKESAKEDRDVERLEREKKDLPKEKENLTERERDQMKRESWNGMEKEVSNNEKESVDASDKLTEQEIVLPEQKKQKEVDSWKNVDREARERRKERDADLEGDRSDKRTKGLDKESNDGCADVEGVMEKEREVYNYSSQHRKRIQRSRGSPQAPNRESFFRSHPQDKDGSQGKVEVSSVVYKVGESMQELIKLWKEHESSQSEMEKNGESSNNGPTLEIRIPSEHVTATNRQVRGGQLWGTDVYTYDSDLVAVLMHTGYCRPTASPPHAAIQELRATVRVLPPQDCYISTLRNNIRSRAWGAAIGCSYRVERCCIVKKGGDTIDLEPCLTHTSTIEPTLAPVTVERTMTTRAAASNALRQQRFVREVTIQYNLCNEPWIKYSISTVADKGLKKPLYTSARLKKGEVLYLETHLSRYELCFTGEKMVKVTPATQLHDPVTEKSQNHHPHSTNGEKNDCENVMIDAFRWSRCKKPLPQKLMRTIGIPLPIEHIELLEENLDWEDVQWSQTGVWIAGKEYTLARVHFLSMN

SEQ ID NO: 35 - DNA, 2418 bp, Glycine max
  CDS (1)..(2418)
atgagtggtgttcctaagagatctcatgaggatgctgttcatcagtcttcaaagcatccacatcaagattcaggtgcatattccaagttgatgccttcagtttcaaatgaccaccatattccttatgatatgagtcaggattcccgggtggcaaagacagtccgtactgaacctcgtgatgcagatagaagatctcctcttcatacagtgtatcggatgccatcatcttcaaatgattctcatgcagatcatcccattggacctgagaacaggatagaatctagggattttaaggagagtagagatccccggtttgagaatcgtgatacgaagacagagaagaaggaattgcatggtgaagccagaagggattctcagattgcaaagagtgagaaggatgtgcgagttgatggcagagaagacgacaacaaggatatcagatatgaacgggatagccataatgattcaaaaggtgacattaagacagacaaggatggctatggtatggtaagcagcagcagccacctgagttggaaagaatcaaaagagtataggggtaagagattttctgatgcccctggtgggagtttggattcctggcatacatcacgtggcaatacacctactgaagttggaaaggacagttcaatggcagaagaaagggactatttggaaacacatgaggctgttggagaaaacaaaattgattctaaaagtgaagatagatttaaagaaagaaaaagaaaggatgtcaagcatcgggattggggggatagggaaaaggagagaagtgatcgcagaagcagtacaccagtaaacaataatagtggtgacaacaaagaatctgccaaggaagatagagatgtagaaaaatgggagaaggagaggaaagatcttccgaaagagaaagaaagttcaaaagagaaggaaaaggataatagcaagagggaatccttgaacggaatggagaaagatggtttgaatgatgggaaggaacttggtgatggatcagcaaaaaatactgagcaagaaaatgtgttgaaacagaaagatgttgatggctggaaaaatgtagatggagaagttagagagaggagaaaagaaagggatgctgatttagaaggagatcgacctgataagcgctgtaaaattgacaagcaatcagaagatggaagtgctcacggggaagggactggagagaaggagagggaagtccataattataatgttcaacatcgtaaaaggatccatcgatcgaggggaagccctcaggtggccaatcgcgaggctcgttttagatctcatactcaagctccagacaatgaagattctgatatttcaggtaaagcagaagtatcttctgttgtttataaagttggtgaaagcatgcaagaattgataaagttgtggaaggcatatgaattatctcaatctcaagtggacaaaaatagtgaaagctctaatagtggccccactcttgaaattcggataccagctgagaatgttacagctacaaaccgtcaagttagaggtggccagctatgggggactgatgtttacacttatgactcagatcttgttgctgttctcatgcatacaggttattgtcgcccaacagcttctccacctcccatggctgtacaagagttgcgcacaaccattcgagtgctacctccgcaagattgctatatttctactctgagaaacaatgtacgttcccgtgcttggggtgctgcaattggttgtagttataaagttgagcggtgctgcattgtaaagaaaggaggtggaactattgatcttgaaccttgccttacacatacctcaactgttgagcctacccttgcaccagtggctattgagcggacaattactactagggctgcagcttcgaatgcattgcggcagcaaagatttgtacgtgaagttacaatacagtacaacctctgcaatgaaccttggatcaaatatagtataagcattgttgctgacaagggtctaaaaaagccactctatacatctgctcgtttaaagaagggagaagttctttatctggagacacactcctgcagatatgagctctgttttactggagagaagatggtgaaggctataccagcaactcagatgcatgacccagatacagagaagtctcaaaatcaccatcaccatcaccatcctgcaaatggtgacaaagctgattctgatgtcatggttgatgtatttcgatggtctcgatgtaagaatcctctaccccagaaactgatgcgcacgattggaatccctctgcctcttgaacatgtggaggtgctagaggaaaacctggactgggaagatgtacagtggtcacaaactggcgtctggattgcaggaaaggaatatacccttgctcgggtgcatttcttgtcaatgaattag

SEQ ID NO: 36 - protein, 805 aa, Glycine max
MSGVPKRSHEDAVHQSSKHPHQDSGAYSKLMPSVSNDHHIPYDMSQDSRVAKTVRTEPRDADRRSPLHTVYRMPSSSNDSHADHPIGPENRIESRDFKESRDPRFENRDTKTEKKELHGEARRDSQIAKSEKDVRVDGREDDNKDIRYERDSHNDSKGDIKTDKDGYGMVSSSSHLSWKESKEYRGKRFSDAPGGSLDSWHTSRGNTPTEVGKDSSMAEERDYLETHEAVGENKIDSKSEDRFKERKRKDVKHRDWGDREKERSDRRSSTPVNNNSGDNKESAKEDRDVEKWEKERKDLPKEKESSKEKEKDNSKRESLNGMEKDGLNDGKELGDGSAKNTEQENVLKQKDVDGWKNVDGEVRERRKERDADLEGDRPDKRCKIDKQSEDGSAHGEGTGEKEREVHNYNVQHRKRIHRSRGSPQVANREARFRSHTQAPDNEDSDISGKAEVSSVVYKVGESMQELIKLWKAYELSQSQVDKNSESSNSGPTLEIRIPAENVTATNRQVRGGQLWGTDVYTYDSDLVAVLMHTGYCRPTASPPPMAVQELRTTIRVLPPQDCYISTLRNNVRSRAWGAAIGCSYKVERCCIVKKGGGTIDLEPCLTHTSTVEPTLAPVAIERTITTRAAASNALRQQRFVREVTIQYNLCNEPWIKYSISIVADKGLKKPLYTSARLKKGEVLYLETHSCRYELCFTGEKMVKAIPATQMHDPDTEKSQNHHHHHHPANGDKADSDVMVDVFRWSRCKNPLPQKLMRTIGIPLPLEHVEVLEENLDWEDVQWSQTGVWIAGKEYTLARVHFLSMN

SEQ ID NO: 37 - DNA, 2394 bp, Triticum aestivum
  CDS (1)..(2394)
atgagcggtgctccaaaaagatcgcatgaggagggtagccattctacacctgcgaaacggcctctggacgatagcagcttgtactcgagcccttctgggaaactcattcaaccaggcggcagtgatttccatggtccttttgaacatgatggaagatttgccaaagtaccacgtgttgagtcacgtgatgataagaggccacctctgacacatcggatgcctgttggctcctccaactttgtggaccacccgacctcatctgacagcagattagaatcaaaacaaaacaaagatgcacgggacaccaaggttgacgaccgggaggcaaaagctgatgctcgggatgtccatagtgatagcaggattgaatttccaggcaataaagctgagactgatgtgaagacaaacaacagagcagatgacactgaaataagagttgaccggagggcgcatggtgatttcacaggtgatgttgtcaaatcggataaggatagccatcctactggaacttcaaacatagcctggaaagataataaagaccatagaggtaaaagatatgttgatcagccagatgatactgcaggatggcgttttcttcgtcctggtatgcaaggcactgatcaaactctcaaggttcaaactattgtggaagagcgcagctccaaggatgcacatgaatctactggtgagaataaaatagaacctaaaagtgaagataagtttagagacaaggacaggagaaagaaagatgaaaaatatagagattttggtgcaagagacgctgatagaaatgatcgcagaattggtagtcagcttgcaggtggtagtgttgaacgaagagaaattcaaagggatgatcgggatgctgaaaaatgggacagggaaagaaaagattcccagaaggacaaggaaaacaatgaccgcgagaaggattctgccaagaaggattcatttgtagcagttgacaaggagaacacaatactggaaaaaacagcttctgatggagctgttaaacctgctgaacatgagagtacagctgctgaaatgaagacacttaaagatgacacatggaaatctcatgatagggatcttaaggacaagaaaagagagaaggatgtggatacaggagacaggcatgaccaaaggagtaaatacaatgacaaagaatctgatgatactggtcctgaaggagatacagagaaagataaggatacttttggaagtatacagcgcaggaggatggcacgcccaaagggaggtagtcaagcatctcaacgggaacctcggttccggtccaaaatgcgtgatggtgaagggtctcaaggtaaatctgaggtatctgcaattgtatataaagctggtgaatgcatgcaagagcttctgaaatcgtggaaagagtttgaagctaccccagatgctagaaatgctgagaatcaacaaaatggtcctactcttgaaattcggatacctgcggagtttgttacttccacgaatcggcaagtaaaaggtgctcagctttggggaacagatgtttatacaaatgattcagaccttgtggctgtgttaatgcatactggttactgctcccccacatcatcacctccaccatctgccatccaagaactgcgtgcaactgttcgtgtgctaccaccacaagacagctatacttcaacactaaggaacaatgtccgttcacgtgcttggggcgctggtattggttgtagcttccgcatagaacgctgctgcattgttaagaaaggtggtggtgccattgatcttgagcctcgccttagccatacgtcagccgtggagcctacactagctccagttgcagtggagcgtacaatgacaacacgagcagcagcttctaatgcattacgtcaacaaagatttgttcgggaagttacaatacagtacaatctctgcaacgagccatggttaaagtacagtataagcattgtggcggacaagggattgaagaagtctctttatacttctgcgaggctgaaaaagggcgaagtcatatacttggaaacacatttcaataggtatgagctgtgcttcagtggggaaaagcctcgctccattggatcaaattccaatgcatctgatttggaaccggaaaaacaccagaacaatagccaccaccatttgcaaaatggagataggggcgccacggaacatgaactccgggacgtgttccgatggtcacggtgtaagaaggccatgcctgaggttgccatgagatccattggtatcccactgccagctgaacaagttgaggtgctgcaggacaatctggagtgggaggatgtgcagtggtcgcagaccggcgtctgggtttctgggaaggagtatccgctcgcccgcgtgcatttcctctcggcgaactag

SEQ ID NO: 38 - protein, 797 aa, Triticum aestivum
MSGAPKRSHEEGSHSTPAKRPLDDSSLYSSPSGKLIQPGGSDFHGPFEHDGRFAKVPRVESRDDKRPPLTHRMPVGSSNFVDHPTSSDSRLESKQNKDARDTKVDDREAKADARDVHSDSRIEFPGNKAETDVKTNNRADDTEIRVDRRAHGDFTGDVVKSDKDSHPTGTSNIAWKDNKDHRGKRYVDQPDDTAGWRFLRPGMQGTDQTLKVQTIVEERSSKDAHESTGENKIEPKSEDKFRDKDRRKKDEKYRDFGARDADRNDRRIGSQLAGGSVERREIQRDDRDAEKWDRERKDSQKDKENNDREKDSAKKDSFVAVDKENTILEKTASDGAVKPAEHESTAAEMKTLKDDTWKSHDRDLKDKKREKDVDTGDRHDQRSKYNDKESDDTGPEGDTEKDKDTFGSIQRRRMARPKGGSQASQREPRFRSKMRDGEGSQGKSEVSAIVYKAGECMQELLKSWKEFEATPDARNAENQQNGPTLEIRIPAEFVTSTNRQVKGAQLWGTDVYTNDSDLVAVLMHTGYCSPTSSPPPSAIQELRATVRVLPPQDSYTSTLRNNVRSRAWGAGIGCSFRIERCCIVKKGGGAIDLEPRLSHTSAVEPTLAPVAVERTMTTRAAASNALRQQRFVREVTIQYNLCNEPWLKYSISIVADKGLKKSLYTSARLKKGEVIYLETHFNRYELCFSGEKPRSIGSNSNASDLEPEKHQNNSHHHLQNGDRGATEHELRDVFRWSRCKKAMPEVAMRSIGIPLPAEQVEVLQDNLEWEDVQWSQTGVWVSGKEYPLARVHFLSAN

SEQ ID NO: 39 - DNA, 2415 bp, Solanum lycopersicum
  CDS (1)..(2415)
atgagtggtactccgaacaaaagacctcacgaggatggtggaaatggtgggagtagtaaccatagttactcttctgctccaaaatactcacatgatgactctggtgcatttcccaaggtgatgagctcaggaacacctgaatatcatgcctcctttgatgtgggccagaatgctcggatgccgaagattcaacggactgaatcttcacgagatgcagatagaagatctcctgtgcttccaatgtaccgtgtctcatcatgtccagttgtttcacatcctgatcattctgttgcttcagaaaataggttggagcccaaggaagttaacaaggacgtcaaggttgagaatcgtgatgccaaaagtgaaataagggagttgtaccaagggactaaatctgacaaggatgatagatttgagaacagagctgatgatggtaaggacattaaaaatagtagggatacttaccctgaatacaagggagatgtgaagacagataaggacaggtttagcggagtgagttggaaagatccgaaagaacagaccaggggaaaaagatatcctgatctccctgttcctgtcgggaacatggatccatggcatgcgtcaagaacccatggtgctgctgagataggaaaagaagtctcaaattctgagaacagggattttgctaaagtgcgtgaagccgttgctgaaaataagatggatttgaaaggtgacgataaatacaaagataaagagagaaaaaggaaagaagggaagcaccgggaatggggagaaagggataaagagagaaatgattgtcggaacaatttacaactagggaatagcacttctgataacaaggaattgcttaaagaggaaagggaatctgagcggtgggagaaggaaagaaatgatctttcgaaggataaggacagaccaaaggactgggaaaaggaccatgcaaagagggaagtgtggaatggagtggagagggaggttttgcagagtgagaaagaagtgattgatgttcctggaaaaacaaacgagccggaaaactcaacagtggagcagaagaaacagaaagatcatgataactggaaaaatactgacagggatggaagtgagaggagaaaggaaagagatactgatttggaaggagagaggcctgagaaacgtgtcaggtgtcatgataaagaaccagaggaaggggacctggatactgaaggaggaggagaaagggaaagagaagcttttaattatggagttcagcagcgcaagagaatgtcgcggccaagagggagccccatggccaatcgcgatcctcgttttaggtcgcacactcatgaaaatgaaggatctcaagtgaagcatgatgtatctgctgtcaattacagagttggtgagtgtatgccagaactgattaaattatggaaggaatatgaatcatccaaagcagatgaagcatctgatagctctccaagtgatcctactctagaaattaggattccagctgaacacgtatcagctacaaatcggcaggtgagaggtggccaactatggggaacagatatatacaccaatgactcggatcttgtcgcagttcttatgcacacaggttactgtcgtacaactgcgtctcctcttttgcctactattacggagttacgtgctactatcagggtactacctccacaaaattgctacatatctactctgaggaacaatgtgcgatcacgtgcgtggggagctgcagttggctgcagctatcgtattgagcggtgctctgttgtgaagaaaggaggtggaacaatcgatcttgaaccttgtctaacacattcctcaaccttggagcctactcttgctccggtggcggtagagcgcactatgaccactcgagctgcagcttcgaatgcactacgacaacagaggtttgtacgtgaagtgacaattcagttcaacttatgcaatgagccttggctcaaatacagtatcagtgttgttgctgacaagggtctaaaaaaggccctttttacatcttcacgcctgaagaagggagaagttctttacttggaaactcattctaagaggtatgagctctgttttagtggtgaaaagatggttaaggctacaacttctctgatgcatgaaatggatgttgacaaacctcaaagtcacaatttacacatggcaaacggagaaaaaaatggagtgaatggtgagaatacgatggtagatatgttccgactgtctcgttgtaagaagcccctgcctcagaaactaatgcaatcagttggaattcctttgccccttgaacatgttgaggttttggaggagaatctggagtgggaaaacattcaatggtcacaaactggtgtttggattgctggaaaagaatatcctcttactagagcgcattttctttccccaaattag

SEQ ID NO: 40 - protein, 804 aa, Solanum lycopersicum
MSGTPNKRPHEDGGNGGSSNHSYSSAPKYSHDDSGAFPKVMSSGTPEYHASFDVGQNARMPKIQRTESSRDADRRSPVLPMYRVSSCPVVSHPDHSVASENRLEPKEVNKDVKVENRDAKSEIRELYQGTKSDKDDRFENRADDGKDIKNSRDTYPEYKGDVKTDKDRFSGVSWKDPKEQTRGKRYPDLPVPVGNMDPWHASRTHGAAEIGKEVSNSENRDFAKVREAVAENKMDLKGDDKYKDKERKRKEGKHREWGERDKERNDCRNNLQLGNSTSDNKELLKEERESERWEKERNDLSKDKDRPKDWEKDHAKREVWNGVEREVLQSEKEVIDVPGKTNEPENSTVEQKKQKDHDNWKNTDRDGSERRKERDTDLEGERPEKRVRCHDKEPEEGDLDTEGGGEREREAFNYGVQQRKRMSRPRGSPMANRDPRFRSHTHENEGSQVKHDVSAVNYRVGECMPELIKLWKEYESSKADEASDSSPSDPTLEIRIPAEHVSATNRQVRGGQLWGTDIYTNDSDLVAVLMHTGYCRTTASPLLPTITELRATIRVLPPQNCYISTLRNNVRSRAWGAAVGCSYRIERCSVVKKGGGTIDLEPCLTHSSTLEPTLAPVAVERTMTTRAAASNALRQQRFVREVTIQFNLCNEPWLKYSISVVADKGLKKALFTSSRLKKGEVLYLETHSKRYELCFSGEKMVKATTSLMHEMDVDKPQSHNLHMANGEKNGVNGENTMVDMFRLSRCKKPLPQKLMQSVGIPLPLEHVEVLEENLEWENIQWSQTGVWIAGKEYPLTRAHFLSPN

SEQ ID NO: 41 - protein, 794 aa, Oryza sativa
MSGAPKRSHEEGSHSTPAKRPLDDSSLYSSPSGKIIQPGSSDFHGSFEHDGRFAKVQRIEPRDDKRPSLAHRMPIGPSNFVDHSISSDGRLESKQNKDPRDTKVDVREAKADTRDVYSDPRVEFPSNKVETDVKTDNRADDNDIRADRRIHADYKGDAKLDKDGHPTAISNIAWKDNKEHRGKRNIEQPSDNADWRFSRPGLQGTDESSKGPVPADERSKDAHESTGENKTEPKTEDKFRDKDRKKKDEKHRDFGTRDNDRNDRRIGIQLGGNSVERRENQREDRDAEKWDRERKDSQKDKEGNDREKDSAKESSVATEKENAILEKTASDGAVKSAEHENKTVEQKTFKDDAWKSHDRDPKDKKREKDMDAGERHDQRSKYNDKESDDTCPEGDIEKDKEALGSVQRKRMARSRGGSQASQREPRFRSRMRDGEGSQGKSEVSAIVYKAGECMQELLKSWKEFEATPEAKSAESVQNGPTLEIRIPAEFVTSTNRQVKGAQLWGTDIYTNDSDLVAVLMHTGYCSPTSSPPPSAIQELRATVRVLPPQDSYTSTLRNNVRSRAWGAGIGCSFRIERCCIVKKGGGTIDLEPRLSHTSAVEPTLAPVAVERTMTTRAAASNALRQQRFVREVTIQYNLCNEPWLKYSISIVADKGLKKSLYTSARLKKGEVIYLETHYNRYELCFSGEKARLVGSSSNAADAETEKHQNSSHHHSQNGDRASSEHELRDLFRWSRCKKAMPESSMRSIGIPLPADQLEVLQDNLEWEDVQWSQTGVWVAGKEYPLARVHFLSSN

SEQ ID NO: 42 - DNA, 21 bp, Artificial Sequence
  primer
caaggactggtgctgagaaag

SEQ ID NO: 43 - DNA, 21 bp, Artificial Sequence
  primer
gcagccaaaatctcaagtagc

SEQ ID NO: 44 - DNA, 20 bp, Artificial Sequence
  primer
tgatccatgtagatttcccg

SEQ ID NO: 45 - DNA, 20 bp, Artificial Sequence
  primer
cagccaaaatctcaagtagc

SEQ ID NO: 46 - DNA, 20 bp, Artificial Sequence
  primer
aaccaaggagaacggaaaat

SEQ ID NO: 47 - DNA, 20 bp, Artificial Sequence
  primer
gccaaggatgtttctgacga

SEQ ID NO: 48 - DNA, 24 bp, Artificial Sequence
  primer
agagtgacagggatgccaagtttg

SEQ ID NO: 49 - DNA, 22 bp, Artificial Sequence
  primer
agcaactctcttccctctatgg

SEQ ID NO: 50 - DNA, 21 bp, Artificial Sequence
  primer
caaggactggtgctgagaaag

SEQ ID NO: 51 - DNA, 21 bp, Artificial Sequence
  primer
ctgctctggtgccacatattc

SEQ ID NO: 52 - DNA, 21 bp, Artificial Sequence
  primer
ctctgcggcaacaaaggtttg

SEQ ID NO: 53 - DNA, 23 bp, Artificial Sequence
  primer
atctgtctccatagcttcatgtg

SEQ ID NO: 54 - DNA, 2757 bp, Artificial Sequence
  codon-optimized HDC1 sequence from A. thaliana
atgagcggcgttccaaagagatcacacgaagagggcgttacgcatccaagctctagctcttcagtggcgaaatacccgcacgaagactctggatcctaccctaagtcgccacatcaacctgttacgccgccaccggctcaggttcatcacaaccatcaacagccgcaccagcatccccaatcccaatcccaatcccaaccacaacctcacctccaagcgcttcctcaccctcattctcactctcactcccattcaccactagctgctgctgcatctgcatctgcaccttatgaggtcgagtcgcgaacggtggttaaagttgcccgtagcgaacccagagatggagagagacgctctccactgccgcttgtctatagatccccatcgctacccacaaccgtttcttctagtgacccgcacttgacacacgccccagttcctatggaacctagagatggtgccaaggacggaagggagataagggtcgagtccagagagaataggagtgacggccgagagatctatggggagacaaagcgagagatacagggtcctaagggcgacagagacgtcaagttcgagagatcagtggatgactttagcggcaagggcaatacggggagttatacgaggaacgacgggagagagatgtacggtgagacgaaacgggagatacaagggccaaagagcgatagggacgccaaattcgagcgacctggggacgattttagcgggaagagtaatgcgggtagctacaccagggacacgaagttcgatcgcgagaaccaaaactacaacgagcaaaagggggagatcaagatggaaaaggaagggcacgcgcacttggcttggaaggagcagaaagactaccatcgagggaagcgcgttgctgaaggatcgactgcaaatgtggacccgtgggttgtaagccgcggaaatccacaaggacccactgaagttgggccaaaagatctctcagctcccgtggaaggctctcacttggaaggacgtgaaaccgtcggagagaacaaagtggacgccaagaacgaggatagatttaaggagaaggacaagaagaggaaggagctaaaacatcgcgagtggggggaccgtgacaaggatagaaacgaccgaagagtctccgtgctcgttggaagcgttatgagcgagccaaaggagattggacgcgaagagagagaatccgatcgctgggaaagggagagaatggagcaaaaggaccgcgaacgcaacaaggagaaggacaaggatcacatcaagcgggaaccaaggactggtgctgagaaagagatctcgcagaacgagaaagagctcggagaagcatctgcaaagccctcggaacaggaatatgtggcaccggagcagaagaagcagaacgagcccgataactgtgagaaggacgaacgcgagacgaaggaaaagaggcgtgaaagggatggagactcagaggcagagagagctgaaaagaggagccggatctccgaaaaggagagcgaagacgggtgtctcgaaggtgaaggagccaccgaaagggaaaaggacgccttcaattatggcgtccagcagaggaaaagagcgctgaggccaagaggaagcccacaaaccactaaccgcgataacgtccgttcacggagtcaagacaacgaaggcgtccaaggcaaaagcgaggtgtcgatcgtcgtatacaaggttggcgaatgcatgcaagagctgatcaagctctggaaggaatacgacttgagccacccggataagagcggcgatttcgccaataatggccccacgctagaagttaggattcccgctgagcatgtgacggctaccaataggcaagtgagaggtggccaactttggggaaccgacatatacaccgacgattccgaccttgtggctgttctcatgcatactggttactgccggccaacagcttctccacctccaccgacaatgcaagagctgagaaccactattagggtcctgccgagccaagattactacacctccaagctgcggaacaatgtccgttctagagcatggggagcgggaataggatgcagttatcgagtcgagcggtgctacatcctgaagaaaggaggtggcacgattgaactggagccctccttaacacactcctcaactgtcgagccaacccttgcaccaatggctgttgagcgatcaatgactacccgtgccgctgcctcgaatgcactccggcaacaaaggttcgtccgagaagtcaccatccaatacaacctctgcaacgagccctggatcaagtactcgattagcatcgtggcggacaagggcctaaagaaacctcttttcacctctgcccgcttgaagaagggggaagttctctacctcgaaacccattcatgccgatacgagctatgtttcgcgggagagaagaccatcaaggccatccaagcctcacaacaacaatcgtcccacgaggctatggagacagacaacaataacaacaagtcgcagaaccatctgacaaacggggacaagacagactcggacaactctctcattgacgtcttccgctggagtcgctgcaaaaagcctctcccgcaaaagctgatgcgaagcatcggatttccactcccggccgatcatatcgaggtgttggaggagaacctggattgggaggacgttcagtggagtcaaaccggagtctggattgctggaaaggagtacaccctggctcgtgtccattttttatccccgaactga

SEQ ID NO: 55 - DNA, 13266 bp, Artificial Sequence
  pTVE704 wheat transformation vector containing the histone deacetylation 1 gene of Arabidopsis, codon optimized for wheat under control of PubiZm, and a bar selectable marker cassette
  promoter (89)..(2085)
  misc_feature (2115)..(4871): codon-optimized HDC1 region for expression in wheat
  3′UTR (4893)..(5153)
aattacaacggtatatatcctgccagtactgggccccctcgagggcgatcgctacgtacctgcaggcccgggttaattaagcggccgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatgcttagggcccggtagttctacttctgtccatgtttgtgttagatccgtgtttgtgttagatccgtgctactagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgacctgaccgggtgatcaccaaaaccatgagcggcgttccaaagagatcacacgaagagggcgttacgcatccaagctctagctcttcagtggcgaaatacccgcacgaagactctggatcctaccctaagtcgccacatcaacctgttacgccgccaccggctcaggttcatcacaaccatcaacagccgcaccagcatccccaatcccaatcccaatcccaaccacaacctcacctccaagcgcttcctcaccctcattctcactctcactcccattcaccactagctgctgctgcatctgcatctgcaccttatgaggtcgagtcgcgaacggtggttaaagttgcccgtagcgaacccagagatggagagagacgctctccactgccgcttgtctatagatccccatcgctacccacaaccgtttcttctagtgacccgcacttgacacacgccccagttcctatggaacctagagatggtgccaaggacggaagggagataagggtcgagtccagagagaataggagtgacggccgagagatctatggggagacaaagcgagagatacagggtcctaagggcgacagagacgtcaagttcgagagatcagtggatgactttagcggcaagggcaatacggggagttatacgaggaacgacgggagagagatgtacggtgagacgaaacgggagatacaagggccaaagagcgatagggacgccaaattcgagcgacctggggacgattttagcgggaagagtaatgcgggtagctacaccagggacacgaagttcgatcgcgagaaccaaaactacaacgagcaaaagggggagatcaagatggaaaaggaagggcacgcgcacttggcttggaaggagcagaaagactaccatcgagggaagcgcgttgctgaaggatcgactgcaaatgtggacccgtgggttgtaagccgcggaaatccacaaggacccactgaagttgggccaaaagatctctcagctcccgtggaaggctctcacttggaaggacgtgaaaccgtcggagagaacaaagtggacgccaagaacgaggatagatttaaggagaaggacaagaagaggaaggagctaaaacatcgcgagtggggggaccgtgacaaggatagaaacgaccgaagagtctccgtgctcgttggaagcgttatgagcgagccaaaggagattggacgcgaagagagagaatccgatcgctgggaaagggagagaatggagcaaaaggaccgcgaacgcaacaaggagaaggacaaggatcacatcaagcgggaaccaaggactggtgctgagaaagagatctcgcagaacgagaaagagctcggagaagcatctgcaaagccctcggaacaggaatatgtggcaccggagcagaagaagcagaacgagcccgataactgtgagaaggacgaacgcgagacgaaggaaaagaggcgtgaaagggatggagactcagaggcagagagagctgaaaagaggagccggatctccgaaaaggagagcgaagacgggtgtctcgaaggtgaaggagccaccgaaagggaaaaggacgccttcaattatggcgtccagcagaggaaaagagcgctgaggccaagaggaagcccacaaaccactaaccgcgataacgtccgttcacggagtcaagacaacgaaggcgtccaaggcaaaagcgaggtgtcgatcgtcgtatacaaggttggcgaatgcatgcaagagctgatcaagctctggaaggaatacgacttgagccacccggataagagcggcgatttcgccaataatggccccacgctagaagttaggattcccgctgagcatgtgacggctaccaataggcaagtgagaggtggccaactttggggaaccgacatatacaccgacgattccgaccttgtggctgttctcatgcatactggttactgccggccaacagcttctccacctccaccgacaatgcaagagctgagaaccactattagggtcctgccgagccaagattactacacctccaagctgcggaacaatgtccgttctagagcatggggagcgggaataggatgcagttatcgagtcgagcggtgctacatcctgaagaaaggaggtggcacgattgaactggagccctccttaacacactcctcaactgtcgagccaacccttgcaccaatggctgttgagcgatcaatgactacccgtgccgctgcctcgaatgcactccggcaacaaaggttcgtccgagaagtcaccatccaatacaacctctgcaacgagccctggatcaagtactcgattagcatcgtggcggacaagggcctaaagaaacctcttttcacctctgcccgcttgaagaagggggaagttctctacctcgaaacccattcatgccgatacgagctatgtttcgcgggagagaagaccatcaaggccatccaagcctcacaacaacaatcgtcccacgaggctatggagacagacaacaataacaacaagtcgcagaaccatctgacaaacggggacaagacagactcggacaactctctcattgacgtcttccgctggagtcgctgcaaaaagcctctcccgcaaaagctgatgcgaagcatcggatttccactcccggccgatcatatcgaggtgttggaggagaacctggattgggaggacgttcagtggagtcaaaccggagtctggattgctggaaaggagtacaccctggctcgtgtccattttttatccccgaactgattgctagcacgcgtggcgcgccgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggaattcgatatcattaccctgttatccctaaagcttattaatataacttcgtatagcatacattatacgaagttatgtttcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctgaaatcaccagtctctctctataaatctatctctctctctataacaatggacccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgagatcacccgttctaggatccgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaaacataacttcgtatagcatacattatacgaagttatatggatctcgaggcattacggcattacggcactcgcgagggtcccaattcgagcatggagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttaaacatcatccgtcggatggcgttgcgagagaagcagtcgatccgtgagatcagccgacgcaccgggcaggcgcgcaacacgatcgcaaagtatttgaacgcaggtacaatcgagccgacgttcacggtaccggaacgaccaagcaagctagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaagtgctgtgcacggatctgccctggcttcaggagatcggaagacctcggccgtccgggcgcttgccggtggtgctgaccccggatgaagtctctagagctctagagggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcagggatcgataccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatccggaaaacgcaagcgcaaagagaaagcaggtagcttgcagtgggcttacatggcgatagctagactgggcggttttatggacagcaagcgaaccggaattgccagattcgaagctcggtcccgtgggtgttctgtcgtctcgttgtacaacgaaatccattcccattccgcgctcaagatggcttcccctcggcagttcatcagggctaaatcaatctagccgacttgtccggtgaaatgggctgcactccaacagaaacaatcaaacaaacatacacagcgacttattcacacgcgaca